(19) United States
(12) Patent Application Publication
(10) Pub. No.: US 2005/0209563 A1
(43) Pub. Date: Sep. 22, 2005

(54) Cassette-based dialysis medical fluid therapy systems, apparatuses and methods

(76) Inventors: Peter Hopping, Lutz, FL (US); Rick Kienman, Tampa, FL (US); Ying-Cheng Lo, Green Oaks, IL (US); Sherwin Shang, Vernon Hills, IL (US); Rafael A. Castellanos, Roselle, IL (US); Dan Marcquenski, Lake Zurich, IL (US); Ted Sorensen, Wonder Lake, IL (US); Tom Johnson, Gurnee, IL (US)

Correspondence Address: Bell, Boyd & Lloyd LLC, P.O. Box 1135, Chicago, IL, 60690-1135, US

(21) Appl. No.: 11/082,147

(22) Filed: Mar. 16, 2005

Related U.S. Application Data
(60) Provisional application No. 60/554,803, filed on Mar. 19, 2004.

(51) Int. Cl. A61M 1/00

(52) U.S. Cl. 604/151; 604/66

(57) ABSTRACT

Improved cassette-based medical fluid therapy systems, apparatuses and methods are provided. In one aspect, various peristaltic pump tubing materials are provided. Cassette and membrane materials, configurations and manufacturing improvements are provided. Various aspects of the invention include: a head height sensing and compensating method and apparatus; an admixing method and apparatus; a pH measuring method and apparatus; an air detecting and removal methods and apparatuses; an active priming apparatus and method; and a volumetric accuracy improvement apparatus and method.

### PRIORITY CLAIM

This application claims priority to and the benefit of U.S. Provisional Patent Application “CASSETTE-BASED DIALYSIS MEDICAL FLUID THERAPY SYSTEMS, APPARATUSES AND METHODS,” Ser. No. 60/554,803, filed Mar. 19, 2004.

### BACKGROUND OF THE INVENTION

In general, the present invention relates to medical fluid delivery systems that employ a pumping cassette. In particular, the present invention provides systems, methods and apparatuses for cassette-based dialysis medical fluid therapies, including but not limited to those using peristaltic pumps and diaphragm pumps.

Due to various causes, a person's renal system can fail. Renal failure produces several physiological derangements. The balance of water, minerals and the excretion of daily metabolic load is no longer possible and toxic end products of nitrogen metabolism (urea, creatinine, uric acid, and others) can accumulate in blood and tissue.

Kidney failure and reduced kidney function have been treated with dialysis. Dialysis removes waste, toxins and excess water from the body that would otherwise have been removed by normal functioning kidneys. Dialysis treatment for replacement of kidney functions is critical to many people because the treatment is life saving.

Hemodialysis and peritoneal dialysis are two types of dialysis therapies used commonly to treat loss of kidney function. Hemodialysis treatment utilizes the patient's blood to remove waste, toxins and excess water from the patient. The patient is connected to a hemodialysis machine and the patient's blood is pumped through the machine. Catheters are inserted into the patient's veins and arteries so that blood can flow to and from the hemodialysis machine. The blood passes through a dialyzer of the machine, which removes waste, toxins and excess water from the blood. The cleaned blood is returned to the patient. A large amount of dialysate, for example about 120 liters, is consumed to dialyze the blood during a single hemodialysis therapy. Hemodialysis treatment lasts several hours and is generally performed in a treatment center about three or four times per week.

Peritoneal dialysis uses a dialysis solution, or “dialysate,” which is infused into a patient's peritoneal cavity via a catheter. The dialysate contacts the peritoneal membrane of the peritoneal cavity. Waste, toxins and excess water pass from the patient's bloodstream, through the peritoneal membrane and into the dialysate due to diffusion and osmosis, i.e., an osmotic gradient occurs across the membrane. The spent dialysate is drained from the patient, removing waste, toxins and excess water from the patient. This cycle is repeated.

There are various types of peritoneal dialysis therapies, including continuous ambulatory peritoneal dialysis (“CAPD”), automated peritoneal dialysis (“APD”), tidal flow APD and continuous flow peritoneal dialysis (“CFPD”). CAPD is a manual dialysis treatment. The patient manually connects an implanted catheter to a drain, allowing spent dialysate fluid to drain from the peritoneal cavity. The patient then connects the catheter to a bag of fresh dialysate, infusing fresh dialysate through the catheter and into the patient. The patient disconnects the catheter from the fresh dialysate bag and allows the dialysate to dwell within the peritoneal cavity, wherein the transfer of waste, toxins and excess water takes place. After a dwell period, the patient repeats the manual dialysis procedure, for example, four times per day, each treatment lasting about an hour. Manual peritoneal dialysis requires a significant amount of time and effort from the patient, leaving ample room for improvement.

Automated peritoneal dialysis (“APD”) is similar to CAPD in that the dialysis treatment includes drain, fill, and dwell cycles. APD machines, however, perform the cycles automatically, typically while the patient sleeps. APD machines free patients from having to manually perform the treatment cycles and from having to transport supplies during the day. APD machines connect fluidly to an implanted catheter, to a source or bag of fresh dialysate and to a fluid drain. APD machines pump fresh dialysate from a dialysate source, through the catheter, into the patient's peritoneal cavity, and allow the dialysate to dwell within the cavity, and allow the transfer of waste, toxins and excess water to take place. The source can be multiple sterile dialysate solution bags.

APD machines pump spent dialysate from the peritoneal cavity, though the catheter, to the drain. As with the manual process, several drain, fill and dwell cycles occur during APD. A “last fill” occurs at the end of CAPD and APD, which remains in the peritoneal cavity of the patient until the next treatment.

Both CAPD and APD are batch type systems that send spent dialysis fluid to a drain. Tidal flow systems are modified batch systems. With tidal flow, instead of removing all of the fluid from the patient over a longer period of time, a portion of the fluid is removed and replaced after smaller increments of time.

Continuous flow, or CFPD, systems clean or regenerate spent dialysate instead of discarding it. The systems pump fluid into and out of the patient, through a loop. Dialysate flows into the peritoneal cavity through one catheter lumen and out another catheter lumen. The fluid exiting the patient passes through a reconstitution device that removes waste from the dialysate, e.g., via a urea removal column that employs urease to enzymatically convert urea into ammonia. The ammonia is then removed from the dialysate by adsorption prior to reintroduction of the dialysate into the peritoneal cavity. Additional sensors are employed to monitor the removal of ammonia. CFPD systems are typically more complicated than batch systems.

Hemodialysis, APD (including tidal flow) and CFPD systems can employ a pumping cassette. The pumping cassette typically includes a flexible membrane that is moved mechanically to push and pull dialysis fluid out of and into, respectively, the cassette. Certain known systems include flexible sheeting on one side of the cassette, while others include sheeting on both sides of the cassette. Positive and/or negative pressure can be used to operate the pumping cassettes.

One problem with the pumping cassettes is leakage. If the flexible membranes experience a pinhole or tear, fluid and/or air can move from one side of the membrane to the other. Movement of fluid from inside the cassette to the inner workings of the machine can damage the machine. Movement of air from the machine or from a bad fluid connection, e.g., an improper supply bag connection, into the cassette can compromise the sterility of the fluid pathways defined by the cassette. There are detection systems that determine when dialysate leaks from the cassette to the machine. It is more difficult, however, to detect air leaking into the cassette. It is therefore important to properly seal the flexible part of the cassette to the rigid part thereof and to properly connect the tubing to the cassette to avoid leaks.

One important feature for cassette-based fluid therapy systems is the material that is used for the cassette and the tubing. The materials have to withstand the rigors of sterilization, be safe for the patient and exhibit good flow properties over an entire therapy. Furthermore, the materials have to withstand rigors that the patient imposes, such as kinking, and varying positive and negative pressures due to a head height difference in elevation between the therapy machine's pump and the location of the patient.

Suitable materials have been developed for fluid systems employing diaphragm-type pumps. A need exists, however, to improve the standard material (e.g., silicone) used in peristaltic pumping. As will be seen below, peristaltic pumping subjects the material to pumping head stresses that are different and in some cases more localized than the stresses mechanically or pneumatically applied to diaphragm pumps. The peristaltic material need exists especially for the tubing section that contacts the peristaltic pump head. Here, the improved tubing materials need to exhibit a proper balance between factors relating to accuracy, resiliency and flexibility.

As alluded to above, one concern with cassette-based fluid pumping systems is patient head height. On one hand, it is desirable to fill and remove fluid to and from the patient as quickly as possible to speed therapy as well as to provide for adequate mixing of the therapy fluid, for example, dialysate. On the other hand, the pressure at the patient has to be maintained within safe limits. While it is important to pump fluid within the positive and negative pressure limits, it is not desirable to sacrifice efficiency by pumping to and from the patient at pressures needlessly away from the safety limits.

The pressure at the pump needs to be controlled so that the pressure at the patient is controlled efficiently and safely. Such control should account for the patient's relative elevation with respect to the therapy machine. If the patient is below the machine, the elevation differences aid the pump in filling the patient but work against the pump in draining the patient. If the patient is elevationally above the machine, the differential aids the machine in draining the patient but hinders the pump in filling the patient. Certain attempts have been made to compensate for pressure caused due to patient head height. At least some of those attempts have involved complicated algorithms that do not produce accurate results repeatedly. Accordingly, a need exists to develop a simple, repeatable and accurate method and apparatus that detects and compensates for patient pressure due to head height in medical fluid pumping systems.

Another concern with cassette-based pumping systems arises when two or more solutions are mixed at the point of use. In one current practice, automated mixing of two different supply solutions is performed by alternatively pumping a small volume of a first solution and the same volume of a separate solution into a mixing reservoir. The two solutions are mixed therein. After multiple volumes of the two solutions have been pumped into the reservoir, a mixed solution is pumped from the reservoir to the patient as a second operation. The solutions are therefore pumped twice, once from the constituent supplies to the mixing reservoir and again from the mixing reservoir to the patient.

In systems employing batch heating of the final solution, the mixing reservoir is logically the heated reservoir because the solution has to be pumped to the heating bag in any case for heating prior to infusion. In systems employing inline heating, however, it is a disadvantage to require a separate pump for the sole purpose of mixing. Accordingly, a need exists for an inline mixing method and an apparatus for mixing two or more different fluids without requiring an additional pump.

A further common problem in peritoneal dialysis systems and cassette-based APD systems in general is the priming of the fluid system. The object of priming APD systems is to push fluid to the very end of the patient line, where the patient connector that connects to the patient's transfer set is located, while not allowing dialysate to flow past the connector and spill out of the system.

Dialysis machines have used gravity to prime. Known gravity primed systems have a number of drawbacks. First, some priming systems are designed for specifically sized bags. If other sized bags are used, the priming system does not work properly. Second, it happens in many systems that at the beginning of priming, a mixture of air and fluid is present in the patient line near its proximal end close to a disposable cartridge or cassette. Dialysate sometimes collects in the cassette due to the installation and/or integrity testing of same. Such dialysate collection can cause air gaps between that dialysate and the incoming priming solution. The air gaps can impede and sometimes prevent gravity priming. Many procedural priming guides, therefore, include the step of tapping a portion of the patient line when the line does not appear to be priming properly. The tapping is meant to dislodge air bubbles trapped in the fluid line. A third problem that occurs relatively often in priming is that the patient forgets to remove the clamp on the patient line prior to priming that line. The clamped patient line will not allow the line to prime properly.

Yet another concern for dialysis systems is volumetric fluid accuracy. For hemodialysis, it is important to remove the necessary amount of ultrafiltrate from the patient so that the patient at the end of therapy achieves what is known as the patient's “dry weight.” For both hemodialysis and PD (referring collectively to CAPD, APD, CFPD, tidal flow systems, etc.), it is important that dialysate is delivered to the dialyzer (hemodialysis) or peritoneal membrane (PD) in a quantity sufficient to remove the requisite amount of impurities from the patients. For PD, since dialysate is delivered to the patient, it is also important that the amount of fluid delivered to the patient's peritoneum is also removed from the peritoneum.

As described herein, the present invention addresses the above-described needs and concerns.

### SUMMARY OF THE INVENTION

The present invention provides medical fluid therapy systems, methods and apparatuses. The present invention in one embodiment relates to a cassette-based peristaltic pumping system. Various features of the present invention however are applicable to other types of pumping systems, such as diaphragm systems. The systems, methods and apparatuses are applicable to a variety of medical fluid therapies, such as peritoneal dialysis (including APD, CAPD, CFPD and tidal flow modalities), hemodialysis, hemofiltration, hemodiafiltration, any type of continuous renal replacement therapy, congestive heart failure therapy as well as others. In particular, the present invention in part provides (i) improved tubing for peristaltic systems, (ii) an improved peristaltic pump, (iii) an improved apparatus and method for measuring and compensating for pressure due to head height, (iv) an improved apparatus and method for inline admixing of multiple different separate constituent solutions (admixing involves the mixing of one or more different solutions, which for a number of reasons, are stored separately from one another), (v) improved valving for cassette-based systems, (vi) improved fluid cassettes, (vii) improved air detection and compensation, and (vii) improved accuracy algorithms.

In one aspect, the present invention provides an improved tubing for a cassette-based peristaltic pumping application. The tubing in one embodiment is part of a peristaltic pump and/or part of a disposable pumping cassette. The tubing is made of one or more materials selected from the group consisting of: high quality silicone, silicone blend, ethylene propylene diene monomer (“EPDM”), polyurethane (“PU”), polyvinylchloride (“PVC”), ultra-high molecular weight PVC (“UHMWPVC”), styrene block copolymer, metallocene-catalyzed ultra-low density polyethylene (“m-ULDPE”), polytetrafluoroethylene (“PTFE”) and any combination thereof. The tubing exhibits many features and characteristics that are desirable for peristaltic pumping. For example, the tubing has a compression set that enables the tubing to rebound after being compressed by peristaltic pump head rollers. The tubing is suitably flexible to be fully compressed by the gear rollers or pump head of the peristaltic pump. The tubing has a suitably consistent diameter and wall thickness and holds its length appropriately. The tubing also exhibits excellent tear resistance and impact resistance during roller head contact.

The tubing is configured to be coupled to an associated pumping cassette in a plurality of different ways, such as via extrusion, molding, extrusion molding, solvent bonding, friction fit, radio frequency sealing, heat sealing and laser welding. The tubing exhibits excellent biocompatibility, low toxicity and extractives. The tubing exhibits a Shore A Hardness in a range of 50% to 85%, has a compression set in the range of 30% to 65% at 73° C. by 22 hours and has a tear resistance in a range of 110 to 480 in-lb per inch. The tubing ages well and has a friction coefficient appropriate to enable the pumping heads or rollers of the peristaltic pump to operate properly.

The above-described advantageous properties and characteristics result in a tubing material that is highly accurate and that offers minimized tubing spallation, rendering low particulate matters (“PM”). In addition to the low PM and accurate flow, the pump tubing of the present invention also achieves and fulfills a broad range of product, functional and application requirements for multiple user environments.

The performance of the tubing meets flow consistency and cyclic fatigue requirements and exhibits a low percentage change in flow over time given a constant pump input. The tubing performs well in a large range of pH values, such as from 1.8 to 9.2. The tubing also performs well in a temperature range of 4° C. to 40° C. The tubing can also be used with extreme pressures due to head height, such as pressures due to ±0.5 m head height differential between the patient and the medical fluid pump, and remains relatively accurate even after a radical shift in pressure due to head height, e.g., +0.5 m to −0.5 m. The tubing is therefore well-suited for use with premixed dialysate or medical fluid as well as with multiple constituent solutions, such as solutions having highly acidic and highly alkaline pH values, which are admixed within the tubing.

Another aspect of the present invention includes a method and apparatus that measures and compensates for patient head height. The method measures the relative head height of a patient connected to a medical fluid pumping apparatus, such as a peritoneal dialysis instrument. The method however is expressly not limited to measuring patient head height but can also be used to measure relative pressures due to head height created by solution bags or drain bags connected to the instrument.

The corresponding head height apparatus includes a sensor that measures the pressure due to head height directly at the machine (at or near the fluid pump) created by the patient, supply bag or drain bag. That measurement is fed to a controller or microprocessor of the medical fluid system to compensate for head height. The operating pressure of the pump, located at or near the sensor, is (i) decreased to ensure that the pressure of the fluid at the patient is within the safety limits of the patient or (ii) increased to maximize fluid flow rate into and out of the patient. The safety of the patient is the primary consideration. However, it is also desirable for efficiency and therapy reasons to control the pressure at the patient as close to the set pressure limits as possible without exceeding such limits.

In the head height apparatus, the cassette includes a chamber, at least one side of which includes a flexible diaphragm or membrane. Fluid is pumped into that chamber. A pressure sensor is mounted on the opposing side of the membrane from the fluid. A vacuum is pulled on the outside (non-fluid side) of the membrane, so that the membrane is sealed or held against the sensing portion of the pressure sensor. Alternatively, the cassette is loaded with very little air between the membrane and the sensor, which creates a slight vacuum between same when the cassette is loaded. The pressure sensor is fixed positionally with respect to the cassette to thereby detect the pressure of fluid within the chamber, either relatively or absolutely.

In the head height method, pressure in one embodiment is measured after fluid has been pumped to the patient and the pump has been stopped, creating a static pressure line. The controller or processor can, if desired, use a simple algorithm to determine and cause the display of the patient's elevation relative to the machine. The operational pressure at the pump is in any case moved either up or down to (i) compensate for the measured pressure due to head height and (ii) control the pressure at which fluid is delivered to the patient.

A third aspect of the present invention includes an inline apparatus and method for mixing two solutions immediately prior to delivering the mixed solution to a patient. The apparatus and method eliminate the need for a mixing pump separate and additional to the fluid supply pump and still provide inline mixing of the solution. The apparatus and method are operable with different types of fluid pumps, such as a peristaltic pump and a diaphragm pump (illustrated below in connection with a peristaltic pump). With a peristaltic pump, a separate chamber is provided and a common flow path between the individual inlets of the different fluids is made through a Y-connection to the chamber. The Y-connection inlets can be coupled fluidly to valves that selectively allow fluid from a first source to enter the flow path and chamber and then alternatingly allow fluid from a second source to enter the flow path and chamber. That cycle can be repeated multiple times.

In one embodiment, to improve the accuracy of the peristaltic pump, a pump motor is coupled directly to a drive plate. The drive plate in turn connects mechanically to either (i) the rollers of the peristaltic pump head or (ii) an assembly housing the rollers. In this configuration, the pump motor positively drives the rollers of the pump head and thus the dialysate through the corresponding tube.

Repeating the above-described cycle, the fluid path immediately preceding the chamber is filled with a new fluid upon each sequencing of the valves. In one embodiment, however, only a portion of the chamber is filled with the newly inputted fluid. In that manner, the two or more fluids mix within the chamber, which can include baffles or other obstructions for facilitating such mixing.

The fluid path preceding the chamber is configured to be, for example, one-half the volume of the chamber. Additionally, the volume of the chamber and/or the flow path is correlated with a known pumping increment, such as a volume achieved from a full revolution of a drive shaft of a peristaltic pump or from a full stroke of a diaphragm pump. Using those correlations, the valves are sequenced in time with, for example, a third of a pump stroke, a half pump stroke, a full pump stroke or multiple full pump strokes of a diaphragm pump or a third of a rotation, a half of a rotation, a full rotation or multiple rotations of a drive shaft of a peristaltic pump. The valves are sequenced in conjunction with the pump operation to pull a precise amount of fluid into the chamber and into the flow path immediately preceding the chamber. Over time, such valve and pump sequencing results in an accurate mixture of fluid that is delivered in mixed form to the patient.

A fourth aspect of the present invention includes multiple improvements to the cassette, including improved sealing ribs, improved flow paths defined by those ribs and an improved method and materials for sealing the flexible membrane to the cassette, namely, a hermetic sonic seal.

A fifth aspect of the present invention includes a method and apparatus for in-line pH sensing. The system uses conductive fittings and a voltage source to place a potential across a portion of the supply fluid flow path. The resulting and measured current through the path is indicative of a pH value of the fluid.

A sixth aspect of the present invention includes an improved apparatus and method for detecting and removing air from the system. An air sensor is placed upstream from a point in the valve arrangement at which the flow of supply fluid is sent either to the patient or to drain. If air is detected, supply flow is diverted to the drain.

A seventh aspect of the present invention includes an improved cassette-based air trap. The air trap is placed in the cassette in a separate zone that also includes the outlet valve to the patient line. The cassette is mounted vertically, with the air trap positioned elevationally above the patient outlet valve. This configuration enables air to collect in the disposable cassette, while allowing dialysate solution to travel through the patient outlet valve to the patient.

An eighth aspect of the present invention includes an apparatus and method for priming the patient line. A sensor is placed in a holder that holds the patient line prior to priming. The sensor senses: (i) if the patient line is in the holder; (ii) if so, that air is present at the fluid priming level; or (iii) if so, that fluid is present at the fluid priming level.

A ninth aspect of the present invention includes improved algorithms for determining volumetric accuracy for positive displacement pump systems, such as diaphragm or peristaltic systems. The algorithms account for variables that effect flow, and which have been ignored traditionally.

Other aspects of the present invention are shown and described herein.

In light of the above-described aspects, one embodiment of the present invention includes a peristaltic pump having a member that is moved across a section of tubing to compress the tubing and thereby move fluid through the tubing, and wherein the tubing has a Shore A Hardness in a range of about 50 to about 85 and a tear resistance of about 110 to about 480 in-lb/in.

The tubing can exhibit (i) a fluid volume accuracy of at least about 90 percent for a fluid having a pH of about 9.0 and after being pumped for at least about 12 hours; (ii) a fluid volume accuracy of at least about 90 percent for a fluid having a pH of about 2.0 and after being pumped for at least about 12 hours; and (iii) a fluid volume accuracy of at least about 90 percent for a fluid being pumped from a head height of at least about ±0.5 meters.

In an embodiment, the tubing has at least one characteristic selected from the group consisting of: (i) a Shore A Hardness in a range of about fifty to about 85; (ii) a tear resistance of about 110 to about 480 in-lb/in, (iii) a substantially uniform diameter; (iv) a substantially consistent wall thickness; (v) is accurate over a temperature range of about 40° C.; (vi) a compression set in a range of about 20% to about 85% at 73° C. and 22 hours; and (vii) is sealable to a disposable cassette.

A second embodiment of the present invention includes a peristaltic pump having a member that is moved across a section of tubing, enabling the tubing to be compressed and expanded, thereby moving fluid through the tubing, and wherein the tubing exhibits a fluid volume accuracy of at least ninety percent for a fluid having a pH of about 2.0 to about 9.0, and wherein the fluid has been pumped through the tubing from a head height of at least about ±0.5 meters for at least 12 hours. The tubing can be installed or sealed to a disposable cassette.

A third embodiment of the present invention includes a peristaltic disposable dialysis apparatus having a disposable cassette defining at least one flow path and at least one valve chamber, wherein the flow path is in fluid communication with a plurality of ports provided by the cassette, and the tubing is in fluid communication with the ports, the tubing forming a loop that operates with a peristaltic pump, and wherein the tubing has a Shore A Hardness in a range of about fifty to about 85 and a tear resistance of about 110 to about 480 in-lb/in. The tubing and the disposable cassette can be sterilized via a process selected from the group consisting of: an ethylene oxide rinse and radiation. The tubing can be mated to the ports via a process selected from the group consisting of: molding, extrusion molding, solvent bonding, friction fitting, radio frequency sealing, heat sealing, laser welding and any combination thereof.

Any of the peristaltic pumps described herein can include a motor that positively drives the peristaltic pump heads.

A fourth embodiment of the present invention includes a disposable dialysis apparatus having a disposable cassette defining at least one flow path and at least one valve chamber, wherein the flow path is in fluid communication with a pair of ports provided by the cassette, and tubing in fluid communication with the ports, the tubing forming a loop that operates with a peristaltic pump, and wherein the tubing exhibits a fluid volume accuracy over at least about twelve hours of at least about ninety percent for a fluid having a pH of about 2.0 to about 9.0, and wherein a fluid pumped through the tubing has been pumped from a head height of at least about ±0.5 meters. The tubing can be mated to the ports via a process selected from the group consisting of: molding, extrusion molding, solvent bonding, friction fitting, radio frequency sealing, heat sealing, laser welding and any combination thereof.

A fifth embodiment of the present invention includes a medical fluid apparatus operable with a fluid pump having a disposable cassette, the cassette including a body and a flexible membrane, the body and membrane defining an enclosed chamber within the cassette, the chamber having a fluid inlet and a fluid outlet, a pressure sensor operably coupled with a portion of the membrane defining the chamber so as to sense pressure fluctuations of a fluid flowing through the chamber, and an electronic control device operable to receive a signal from the pressure sensor indicative of a pressure due to head height of a patient and use the signal to determine pressure at which to operate the pump. The desired pressure is a function of at least one of: maximizing flow rate and operating within at least one established pressure limit. The electronic control device is operable to determine the operating pressure based on the pressure signal and a factor corresponding to a predicted pressure drop due to at least one flow restriction between the pump and the patient. The pressure sensor and the electronic control device can be housed in a unit that is coupled with the disposable cassette and houses a driving mechanism of the pump, which can be activated mechanically or pneumatically. The pump can be a peristaltic pump, wherein the cassette includes a tube that is coupled operably with a driving mechanism of the peristaltic pump. The chamber can be a pumping chamber of the pump.

In an embodiment, when the pressure due to head height is positive, the electronic control device is operable to set a positive operating pressure at the pump higher than a desired positive pressure at the patient to fill the patient at the desired positive pressure, and to set a negative operating pressure at the pump lower than a desired negative pressure at the patient to drain the patient at the desired negative pressure. In another embodiment, when the pressure due to height pressure is negative, the electronic control device is operable to set a positive operating pressure at the pump lower than a desired positive pressure at the patient to fill the patient, and to set a negative operating pressure at the pump lower than a desired negative pressure at the patient to drain the patient at the desired negative pressure.

A sixth embodiment of the present invention includes a medical fluid apparatus having a driving mechanism of a pump, a pressure sensor, and an electronic control device operable to receive a signal from the pressure sensor indicative of a pressure due to head height of a patient and use the signal to determine an operating level at which to operate the driving mechanism. The operating level can be a function of at least one of: maximizing flow rate and operating within at least one established pressure limit. The pump can be a peristaltic pump and the driving mechanism includes a head that rotates against a fluid carrying tube, and wherein the operating level is a level at which the head rotates against the tube.

A seventh embodiment of the present invention includes a medical fluid apparatus operable with a fluid pump that pumps a fluid volume V per pumping increment having a mixing chamber, first and second fluid supplies holding different first and second fluids, a fluid path having a first end fluidly connected to an inlet of the chamber, the fluid path having a volume P, which is a predetermined portion of the volume V, and first and second valves placed at a second end of the fluid path and controlling flow of the first and second fluids, the valves alternated so that (i) a volume P of the first fluid is pumped to the flow path and a volume V-P of the first fluid is pumped to the mixing chamber in a first pumping increment and (ii) a volume P of the second fluid is pumped to the path and a volume V-P of the second fluid is pumped to the mixing chamber in a second pumping increment.

The mixing chamber can also be a pumping chamber of the fluid pump. The pump is (i) of a type selected from the group consisting of: a diaphragm pump and a peristaltic pump; (ii) a peristaltic pump and the chamber and fluid path are located upstream of the pump; and (iii) a peristaltic pump and the chamber and fluid path are located downstream of the pump.

The volume P can be at least substantially one-half or equal to the volume V. The valves can be controlled to produce a mixture of the first and second fluids in other than a one-to-one ratio. The first increment can constitute a first percentage of a complete pump cycle and the second increment can constitute a second percentage of the pump cycle, the first and second percentages chosen to create a desired overall ratio of first and second fluids. If the pump is a peristaltic pump, the pump increment can be: (i) a portion of a revolution of a drive shaft or roller during which at least one roller compresses a fluid tube; (ii) a full revolution of the drive shaft; and (iii) multiple revolutions of the drive shaft or roller.

An eighth embodiment of the present invention includes a medical fluid apparatus having a mixing chamber, first and second fluid supplies holding different first and second liquids, the supplies in fluid communication with the mixing chamber, a fluid pump, and first and second valves controlling flow of the first and second liquids, the valves and the pump operable to alternatingly partially fill the chamber with the first fluid and then partially fill the chamber with the second fluid and simultaneously remove some but not all of the first fluid from the chamber. The apparatus includes a pressure sensor coupled operably to a flexible membrane portion of the mixing chamber, the sensor measuring a pressure due to a relative head height position between the pump and a patient fluid connection. The first and second supplies can be tied together to a common inlet fluid path running to the mixing chamber.

A ninth embodiment of the present invention includes a medical fluid system having a disposable cassette defining multiple flow paths, multiple valve chambers and multiple fluid ports, a tube connected to one of the fluid ports, the tube including a conductive portion, the conductive portion operable to enable a reading indicative of the pH value of a fluid traveling within the tube to be taken, and a processor operable to input the reading and determine if the pH value for the fluid is acceptable. The conductive portion can include a conductive fitting coupled to at least one section of the tube. The apparatus can include a housing that encloses the processor and accepts the cassette, wherein the housing includes a coupler operable to receive and hold the conductive portion.

A tenth embodiment of the present invention includes a medical fluid apparatus having a disposable cassette defining multiple flow paths, multiple valve chambers and multiple fluid ports, a supply container connected fluidly to a first one of the fluid ports, a drain line connected fluidly to a second one of the fluid ports, a patient fill line connected fluidly to a third one of the fluid ports, a peristaltic pump operable to pump fluid from the supply bag to the patient fill line or the drain line based on which valve chambers are opened and closed, and an air sensor positioned relative to the valve chambers so that fluid from the supply container can be diverted to drain instead of being pumped to the patient if air in the fluid is detected by the air sensor. The air sensor can be: (i) positioned directly upstream to or downstream from the peristaltic pump; (ii) coupled operably to the cassette; (iii) coupled operably to a supply line connecting the supply container to the cassette; and (iv) a first air sensor, and which includes a second air sensor coupled operably to the patient fill line.

An eleventh embodiment of the present invention includes a medical fluid system having a disposable cassette defining multiple flow paths, multiple valve chambers and multiple fluid ports, a peristaltic pump connected fluidly to the cassette, a patient line connected to one of the fluid ports, a patient line holder into which the patient line is placed and held, a sensor cooperating with the holder to send a signal indicating (i) that the patient line has not been placed in the holder, (ii) that the patient line has been placed in the holder and fluid has not yet reached a sensible level, and (iii) that the patient line has been placed in the holder and fluid has reached a sensible level, and a controller or processor operable to input the signal and make at least one determination based on the signal. The system can include a connector placed at the end of the patient line, the connector aiding a person to position the tube properly in the holder, wherein the sensor is optical, ultrasonic, capacitive or inductive and includes multiple holders organized to aid a person to properly initiate therapy.

A twelfth embodiment of the present invention includes a system for improving the volumetric accuracy in dialysate pumping. The system includes an apparatus that: (i) identifies a factor causing volumetric error in pumping dialysate; (ii) isolates the factor and empirically determines a relationship between the factor and volume of dialysate pumped; (iii) determines a constant K for the factor using the empirically determined relationship; and (iv) modifies an overall equation for calculating a volume of dialysate pumped by a product of the constant K and a value for the factor. The value for the factor can be: (i) measured or entered; (ii) for a diaphragm pumping system selected from the group consisting of: a position of a pump diaphragm, a pressure differential across the diaphragm, a material for the diaphragm, stress and strain characteristics of the diaphragm and any combination thereof; or (iii) for a peristaltic pumping system selected from the group consisting of: inlet pressure to a peristaltic pumping tube, outlet pressure to the peristaltic pumping tube, material of the peristaltic pumping tube, tubing temperature, pumping head wear, tubing dimensions and any combination thereof.

Other embodiments of the present invention are shown and described herein.

It is therefore an advantage of the present invention to provide improvements for dialysis and other medical fluid therapy treatments.

It is another advantage of the present invention to provide methods for improving the accuracy of fluid pumping systems.

Still a further advantage of the present invention is to provide methods and apparatuses for improving the reliability and durability of fluid pumping systems.

Other advantages of the present invention are to provide an improved tubing, improved cassette material, improved membrane material, improved membrane configuration and manufacturing method for a cassette-based peristaltic pumping system.

Furthermore, it is an advantage of the present invention to provide an apparatus and method that senses and compensates for pressure due to head height.

Further still, it is an advantage of the present invention to provide a method and apparatus for admixing multiple different solutions in an inline fashion without requiring an additional mixing pump.

Another advantage of the present invention is to provide an inline pH detection apparatus and method for a cassette-based peristaltic pumping system.

Moreover, it is an advantage of the present invention to provide an air detection and removal apparatus and method for a cassette-based peristaltic pumping system.

Additionally, it is an advantage of the present invention to provide an active priming apparatus and method for a cassette-based peristaltic pumping system.

Yet another advantage of the present invention is to provide a method for improving volumetric accuracy for a positive displacement pump medical fluid system.

Additional features and advantages of the present invention are described in, and will be apparent from, the following Detailed Description of the Invention and the figures.

### BRIEF DESCRIPTION OF THE FIGURES

FIG. 1 illustrates the system of the present invention connected fluidly to a patient.

FIG. 2 is a perspective view of one embodiment of an actuator unit with a pump and valve cassette installed.

FIG. 3 is a perspective view of the actuator unit of FIG. 2 with the cassette removed.

FIGS. 4 to 7 are various perspective views of one embodiment of a cassette having a peristaltic pumping portion.

FIG. 8 is a top plan view of a peristaltic pump used in one embodiment of the present invention.

FIG. 9A is a sectioned elevation view taken along line IX A-IX A of FIG. 8.

FIG. 9B is a sectioned elevation view of one embodiment of a positive drive peristaltic pump of the present intention.

FIG. 9C is a perspective view of the groove plate of FIG. 9B.

FIG. 9D is a section of FIG. 9C showing the drive stop of the groove plate of FIG. 9B.

FIGS. 9E and 9F are elevation views of alternative embodiments of positive drive peristaltic pumps of the present intention.

FIG. 9G is an exploded perspective view of a roller assembly of the positive drive peristaltic pump of FIG. 9F.

FIGS. 10 and 15 illustrate various embodiments of a flexible membrane that covers the valve and flow path chambers of the cassette.

FIGS. 11A and 11B illustrate sectioned views of a valve actuator uncoupled and coupled respectively to a flexible membrane.

FIGS. 12 to 14 illustrate one apparatus and method for mechanically locking the membrane to the cassette.

FIG. 16 illustrates an interface between a pressure sensor, the membrane and the cassette.

FIG. 17 is a schematic process flow diagram illustrating one embodiment of a method for controlling pump pressure via head height sensing.

FIG. 18 is a rear perspective view of the actuator unit shown in FIGS. 2 and 3, which shows conductive fittings and a patient fluid line holder.

FIG. 19A is an electrical circuit operable with the conductive fitting of FIG. 18.

FIG. 19B is a graph illustrating results from experiments using the conductive fitting of FIG. 18.

FIGS. 20, 21, 22, 23A, 23B and 23C are schematic flow diagrams illustrating various embodiments of an air detection and removal apparatus and method of the present invention.

FIGS. 24A to 24C illustrate schematic views of various embodiments of a zoned flow path and valve arrangement for the disposable cassette of the present invention.

FIGS. 25 and 26 are top and side sectioned elevation views, respectively, of a patient tube holder and associated apparatus for priming the patient line.

### DETAILED DESCRIPTION OF THE INVENTION

The present invention relates to medical fluid delivery systems that employ a pump, such as a peristaltic pump. In particular, the present invention provides systems, methods and apparatuses for cassette-based dialysis therapies including but not limited to hemodialysis, hemofiltration, hemodiafiltration, any type of continuous renal replacement therapy (“CRRT”), congestive heart failure treatment, CAPD, APD (including tidal modalities) and CFPD. The cassette is disposable and typically discarded after a single use or therapy, reducing risks associated with contamination.

### The Medical Fluid Therapy System Generally

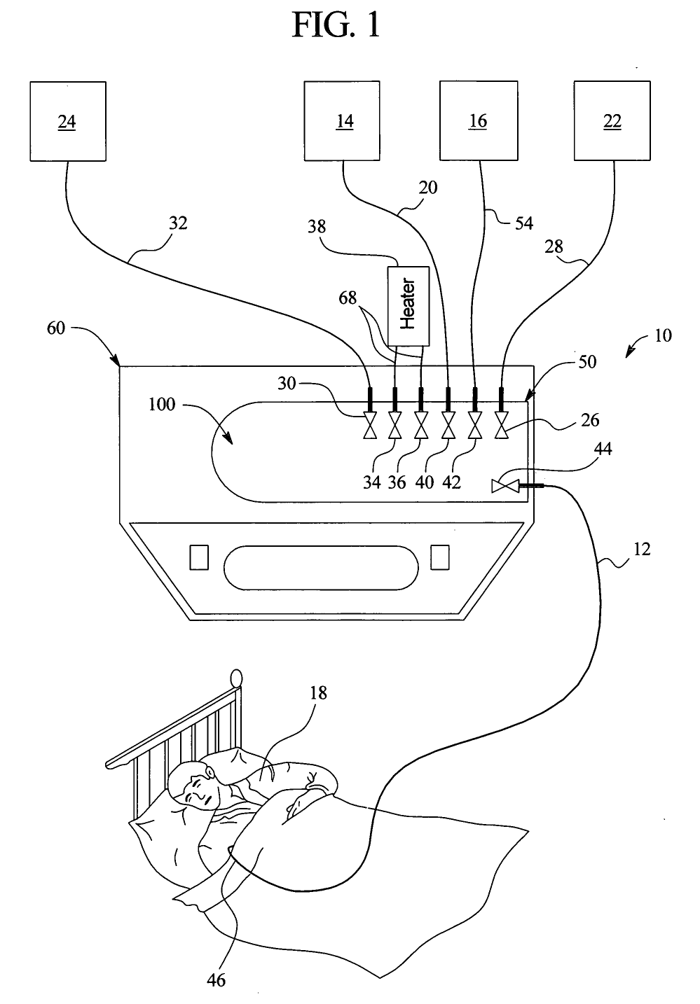

Referring now to the drawings and in particular to FIG. 1, the teachings of the present invention, while applicable to each and all of the above-mentioned types of therapies, are described for ease of illustration by a peritoneal dialysis system 10. FIG. 1 shows system 10 in operation with a patient 18. Subsequent figures discuss the details of the primary components of system 10, namely, a disposable cassette or cartridge and an instrument that operates with the cartridge. As will become apparent, the peristaltic type of system illustrated is not critical to the teachings of the present invention in many cases and such teachings are readily applied to different types of medical fluid therapy systems known to those of skill in the art.

As discussed in more detail below, system 10 includes a disposable cassette or cartridge 50. Cassette 50 includes or defines fluid paths, valves chambers and a peristaltic pump tube and rollers. An instrument or actuator unit 60 operates the valves and pump to control the amount of fluid delivered to and removed from the patient 18.

A cassette-based system 10 controllably and selectively pumps exchange fluid volumes through lines 12, 20, 32, 28 and 54 between patient 18 and bags 14, 24, 22 and 16, respectively. Tube 12 is provided from cassette 50 to administer and remove exchange volumes of fluid, such as dialysate, to and from patient 18. Supply reservoir or bags 14, 16 and 22 contain supply dialysate volumes to be administered to patient 18.

Bags 14, 16 and 22 can be of any suitable size, such as six liters each. Bags 14, 16 and 22 are connected fluidly to cassette 50 via lines 20, 54 and 28, respectively. A recovery reservoir 24 recovers used or spent dialysate from patient 18. A system controlled valve 26 is connected fluidly to line 28, which is connected to reservoir 22. A system controlled valve 30 is connected fluidly to line 32, which is connected to spent fluid reservoir 24. Valve 30 controls flow to spent reservoir 24 and prevents used dialysate from being released accidentally from recovery reservoir 24.

In the illustrated embodiment, cassette 50 of system 10 includes or defines seven valves 26, 30, 34, 36, 40, 42 and 44. Valves 26, 30, 40, 42 and 44 control fluid flow from bags 14, 16, 22 to patient 18 and back to bag 24 and one or more of bags 14, 16 and 22. Supply bags 14, 16 and 22 can double as drain or waste bags, cooperating with bag 24. Valves 34 and 36 control fluid flow to heater 38. Once heated dialysate fluid is delivered via line 12 to the peritoneal cavity of patient 18, waste and toxins are transferred across the patient's peritoneal membrane to the dialysate in a manner that is well known.

The above-described fluid communication enables one or more fluid exchanges in the peritoneal cavity to take place. During a first volume exchange, pump 100 may remove an initial volume of liquid from patient 18 and pump that volume to the initially empty reservoir bag 24. In one embodiment, drain bag 24 is sized to receive all spent fluid from patient 18 (beginning from bags 14, 16 and 22), isolating fresh tubes from the spent fluid tube 32.

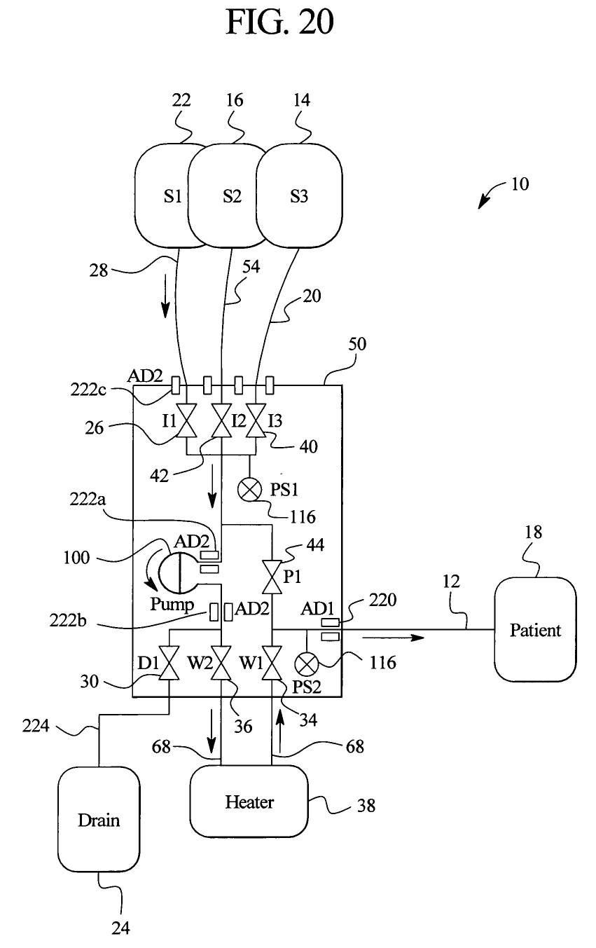

The direction of fluid flow is controlled by valves 26, 30, 40, 42 and 44, the tubing, the cassette pathways and pump 100. Pump 100 refers to the drive or instrument portion of the pump as well as the tubing and cassette portion 78 shown below. Pump 100 in one embodiment is driven in a single direction for both the pump-in and pump-out cycles of the therapy. In that case, valves 26, 30, 40, 42 and 44 switch to direct the flow of fluid from the correct source to the correct destination. Alternatively, pump 100 pumps in the opposite direction in cooperation with valves 26, 30, 40, 42 and 44 to pump spent dialysate from patient 18. FIGS. 20, 21, 22, 23A and 23B and associated text provide a good description of one embodiment for switching the valves to perform the pump-in and pump-out cycles of the therapy.

In either case, once inside the peritoneal cavity, waste and toxins are transferred to the exchange volume across the patient's peritoneal membrane in a manner that is well known. In either case, when delivering fluid to patient 18, the fluid, via valves 34 and 36 is pumped though inline heater 38. Inline heater 38 can be an electrical plate heater, an infrared heater, a convective heater, a radiant heater and any combination thereof. One control scheme for controlling heater 38 is described and claimed in U.S. Ser. No. 10/155,560, entitled Method and Apparatus for Controlling a Medical Fluid Heater, the entire contents of which is incorporated herein by reference.

System 10 in one embodiment employs a peristaltic pump 100 that can pump at a flowrate of zero to about five hundred milliliters/minute. Pump 100 can pump from each of the supply bags 14, 16 and 22 sequentially or, in the case of admixing, from two or more of bags 14, 16 and 22 simultaneously. The valves used to determine which supply bags are active are actuated selectively and automatically via mechanical, electrical, electromechanical or pneumatic actuators, which are housed in unit 60.

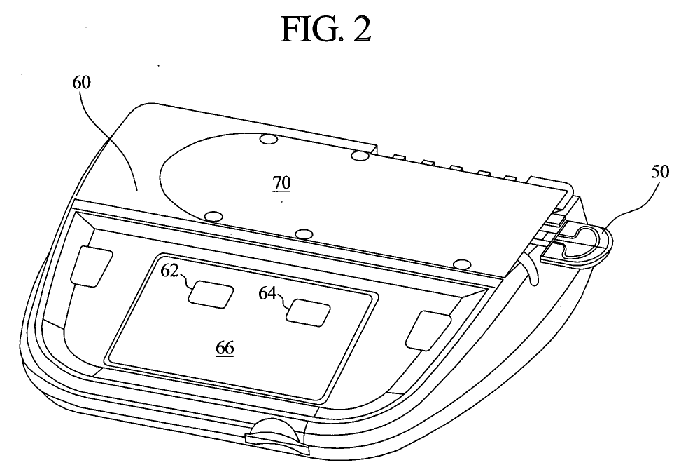

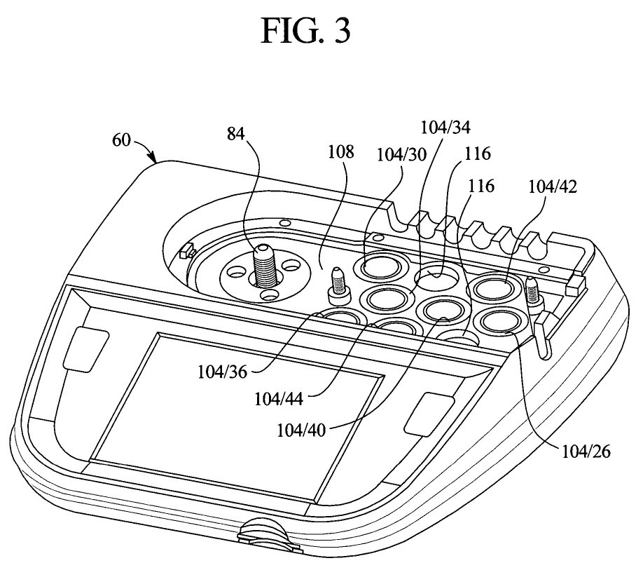

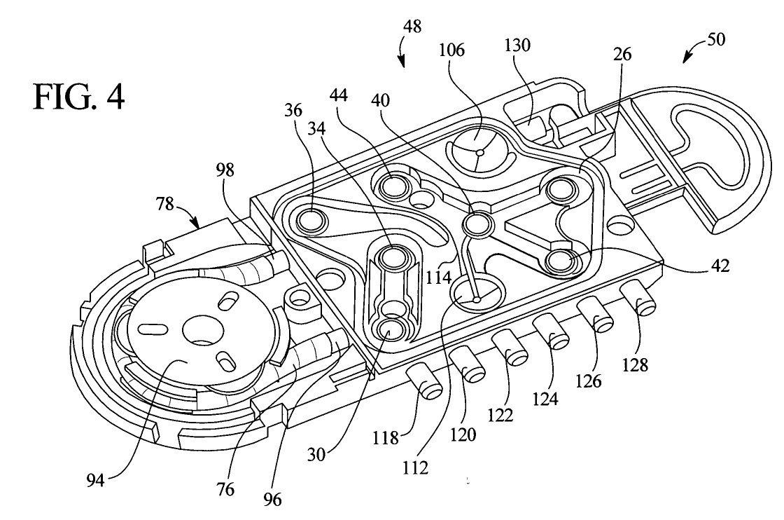

Referring now to FIGS. 2 and 3, various views or portions of actuator unit 60 are illustrated. As seen in FIG. 2, unit 60 operates with a disposable cassette 50. In FIG. 3, cassette 50 is removed to expose some of the actuators within unit 60. In use, disposable cassette 50 is placed in and is operably coupled to motor/valve actuator unit 60. FIGS. 4 to 7 illustrate cassette 50 in more detail. The user (patient or caregiver) controls operation of motor/valve actuator unit 60 via controls 62 and 64 and display panel 66, which can operate with a touch screen or touch pad. Actuator 60 can also employ voice guidance and/or voice activation.

A moveable lid 70 holds cassette 50 in place against a surface 108 of motor/valve actuator unit 60, allowing disposable cassette 50 to be installed for a session of therapy and discarded thereafter. To that end, cassette 50 in one embodiment is made of plastic or other suitable disposable material that is readily sterilized.

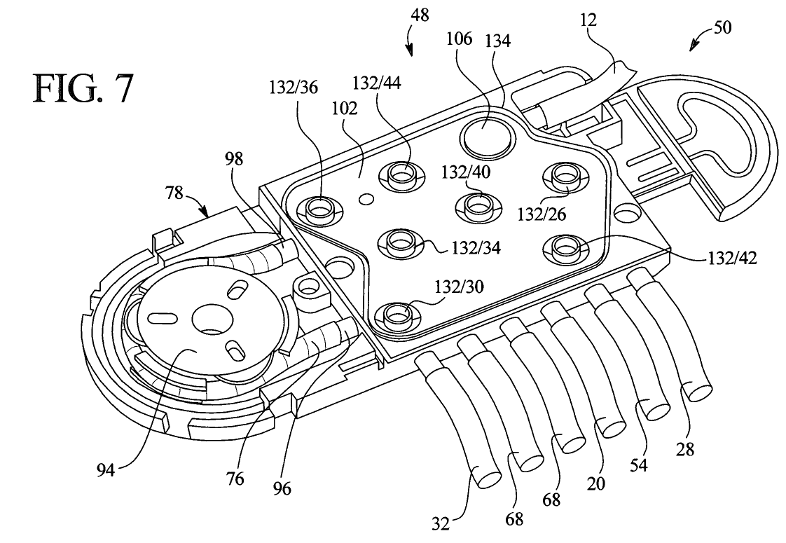

As seen in the underside view of cassette 50 in FIG. 7, tubes 32, 68, 20, 54, 28 and 12 are provided with the disposable cassette 50 in one embodiment and are attached to bag 24, heater 38, supply bags 14, 16 and 22 and patient 18, respectively, prior to therapy via any suitable apparatus and method. To that end, clamps and/or tip connectors/protectors can be provided with one or more or all of tubes 32, 68, 20, 54, 28 and 12 to facilitate sterile fluid connection to the various peripherals.

FIG. 4 illustrates that cassette 50 defines or includes bulkhead ports 118 to 130. Those ports are coupled to tubes 32, 68, 20, 54, 28 and 12, respectively. Cassette 50 and ports 118 to 130 are acrylic in one embodiment. Tubes 32, 68, 20, 54, 28 and 12 are ultra-high molecular weight polyvinyl chloride (“UHMWPVC”) in one embodiment. Those materials are readily solvent bonded via a solvent, such as cyclohaxanone and methyl ethyl ketone (MEK). The solvent partially dissolves surfaces of the ports and tubes so that a chemical bond is formed between same, yielding a strong connection, which is hermetic and sterilized readily.

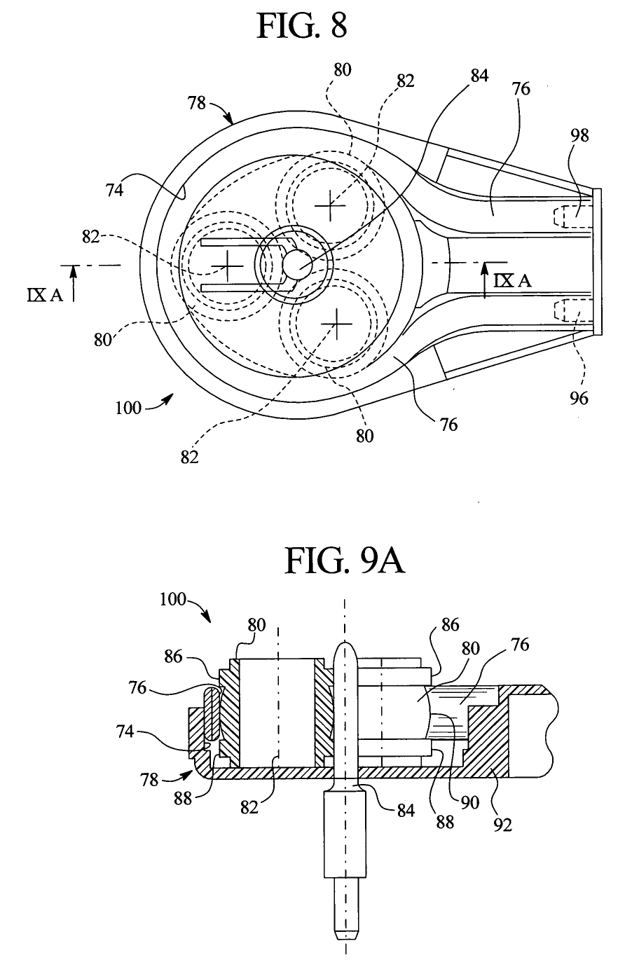

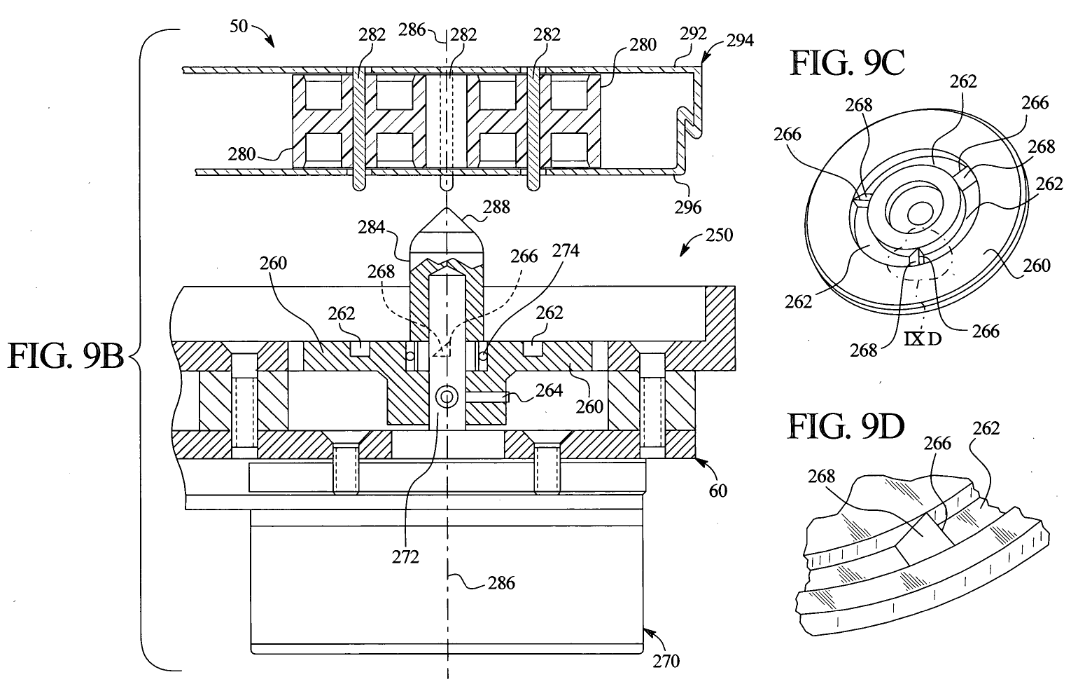

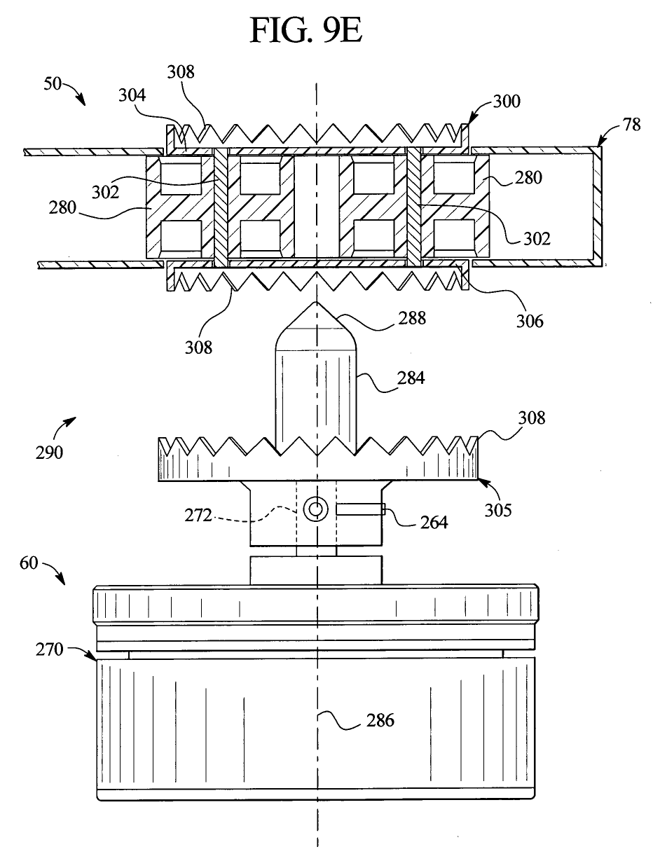

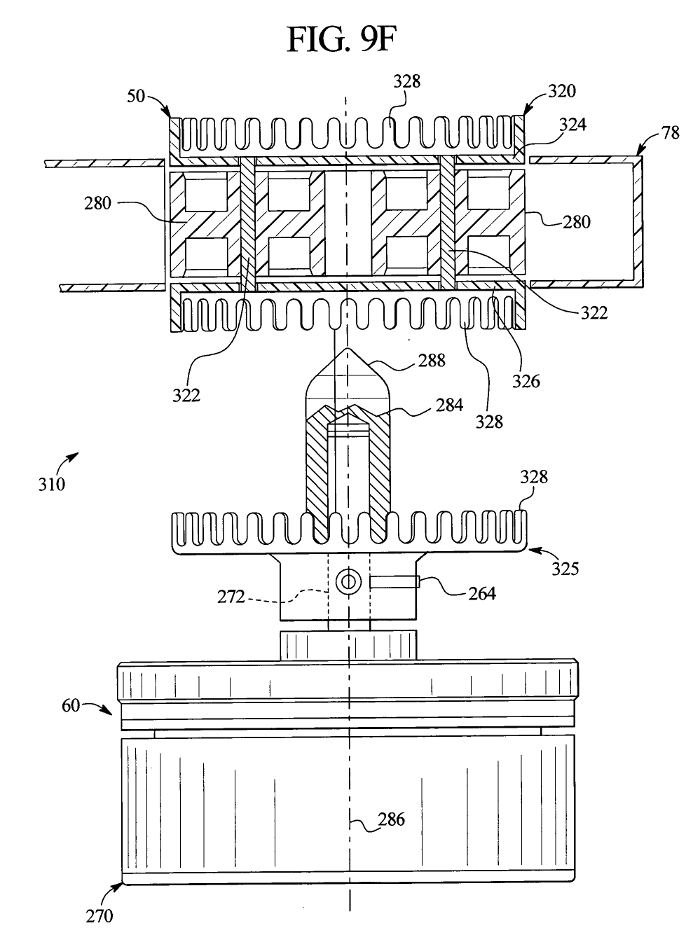

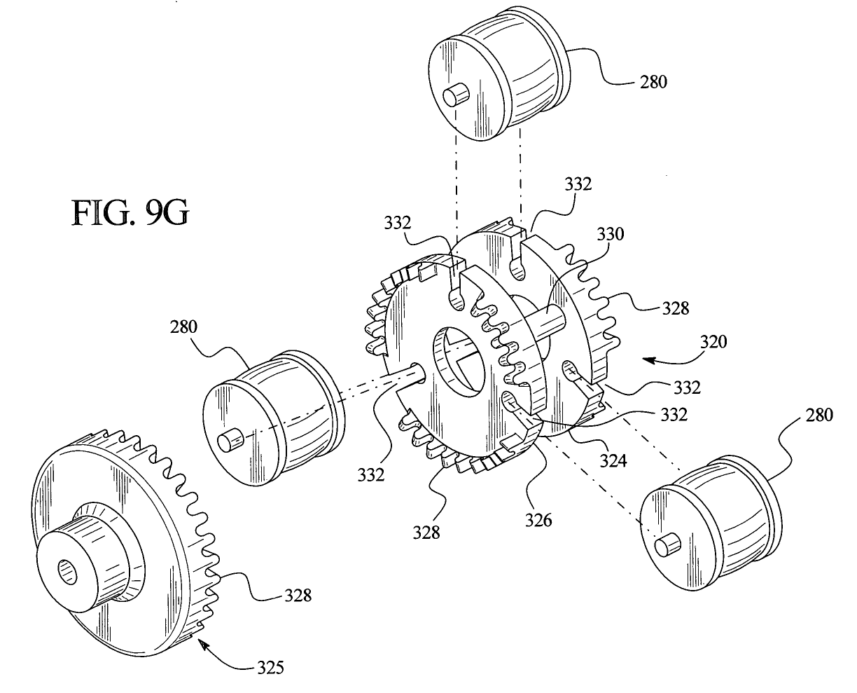

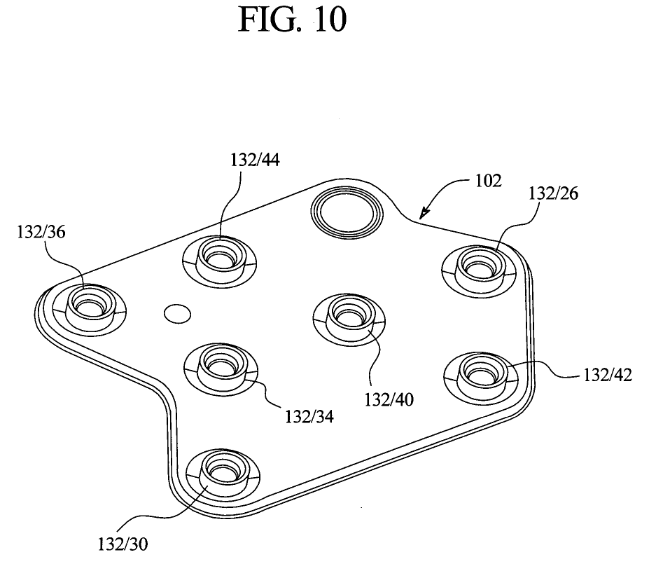

Tubes 32, 68, 20, 54, 28 and 12 extend outside of cassette 50. As seen in FIGS. 4, 7, 8 and 9A, a deformable tube 76 extends inside of cassette 50. FIGS. 8 and 9A highlight a pump casing portion 78 of cassette 50 shown also in FIGS. 4 and 7. FIG. 9A is a sectioned view of FIG. 8 taken along line IX A-IX A in FIG. 8.

Tube 76 forms a loop that is approximately planer and parallel with respect to the remainder of cassette 50. The external surface of the looped portion of tube 76 contacts and bears on a similarly shaped support surface 74, which is provided in the pump casing 78 of cassette 50. Surface 74 cooperates with pump rollers 80 to compress tube 76.

### Peristaltic Pump Roller Materials

Pump 100 includes a variable number of pump rollers 80 (three shown). Rollers 80 are made of any suitable metal, plastic, composite or other material and in one embodiment are made of a fiber reinforced material. In one embodiment, the material is fiber reinforced polyacetal (“POM”). In another embodiment, the material is fiber reinforced high density polyethylene (“HDPE”). The fiber can be carbon, stainless steel, KEVLAR®, ultra-high molecular weight polyethylene and any combination or derivative thereof. The fiber can be supplied in any proportion to the base material, for example from one to fifty percent by mass.

Each roller 80 rotates about an independent axis 82 and is located inside the loop formed by deformable tube 76. In one embodiment, the rollers and associated linkages (FIGS. 8 and 9) are identical to one another. A drive spindle or shaft 84 rotates about an axis that is substantially parallel to the axes 82 about which rollers 80 rotate. Drive shaft 84 separates or pushes the rollers 80 outward when the shaft is inserted between the rollers. Drive shaft 84 acts as a wedge that forces the rollers 80 outward, thereby compressing deformable tube 76 against support surface 74 of portion 78 as seen in FIG. 9A.

Drive shaft 84 drives rollers 80 against tube 76 and surface 74 by friction. In one embodiment, each roller 80 includes, about axis 82, cylindrical rings 86 and 88 that contact and create friction with drive shaft 84. A bearing surface 90 of roller 80, which in one embodiment is convex or barrel-shaped, contacts and compresses tube 76. In another embodiment, bearing surfaces 90 are smooth and substantially cylindrical or straight in cross-section.

Pump casing 78 of peristaltic pump 100 includes a base 92 (FIG. 9A) and a cover (not illustrated) that snap-fits or press-fits onto base 92. Cassette 50 also includes or defines hollow male elements 96 and 98 to which deformable tube 76 is connected sealingly via a suitable method, such as press-fitting or solvent bonding.

As seen in FIGS. 4 and 7, in one embodiment rollers 80 are housed inside housing 94. Housing 94 separates rollers 80 from one another and holds each roller 80 in a rotatably fixed manner. Housing 94 and rollers 80 are dimensioned so that when drive shaft 84 is not present, tube 76 pushes rollers 80 inward towards each other. That is, without a counteracting force, tubing 76 elastically pushes rollers 80 inwards. In the illustrated embodiment, pump 100 relies on adequate friction between the rollers 80 and tubing 76 for proper movement of rollers and proper pumping of the medical fluid. While three rollers 80 are illustrated, any suitable number of rollers 80 may be provided.

As seen in FIGS. 8 and 9A, inserted drive shaft 84 (seen also in FIG. 3) separates rollers 80 radially from one another and completely compresses tubing 76 between the casing bearing surface 74 and roller support surfaces 90. Such compression completely occludes tubing 76 in multiple places at each of the rollers 80. The compression also exerts a force on rollers 80 and drive shaft 84. Therefore, when drive shaft 84 is rotated by a motor located in motor/valve actuator unit 60, rollers 80 are driven by friction against tubing 76. As stated above, the direction of rotation of shaft 84 and rollers 80 may or may not be reversible and may or may not be determine the direction in which fluid is pumped.

### Positive Drive Engagement and Disengagement for Peristaltic Pump

Referring now to FIGS. 9B to 9D, one embodiment of a direct or positive drive peristaltic pump 250 is illustrated. As discussed above, pump 100 relies on adequate friction between the rollers 80 and tubing 76 for proper movement of the rollers and proper pumping of the medical fluid. Pump 100 simplifies the loading and unloading of the disposable cassette 50 to and from unit 60. The friction drive of pump 100, however, may result in: (i) the generation and accumulation of particles (“PM”) in cassette 50 and unit 60; and/or (ii) a lack of accuracy due to friction slippage between drive shaft 84 and rollers 80 and/or rollers 80 and tube 76. Direct drive pump 250 helps to eliminate the PM and potential inaccuracies.

Pump 250 includes a variable number of pump rollers 280, e.g., three. Rollers 280, as above, are made of any suitable metal, plastic, composite or other material, and in one embodiment are made of a fiber reinforced material. Rollers 280 are located inside a loop formed by the deformable tube 76 (illustrated in FIGS. 4, 7 and 8). Each roller 280 rotates about a pin 282. Pin 282 may be made of a self-lubricating material and function also as a cylindrical bearing. Alternatively, roller or ball bearings (not illustrated) may be placed between rollers 280 and pins 282 to reduce friction. Further alternatively, pins 282 may be integral to and rotate with rollers 280. In one embodiment, rollers 280 are identical to one another as are pins 282. Rollers 280 and pins 282 are spaced apart evenly, e.g., at 120 degrees, about centerline 286.

Shaft 84 of pump 100 above is a drive spindle that rotates rollers 80 via friction. Shaft 284 of the instant pump 250, on the other hand, does not drive rollers 280. Instead, shaft 284 acts as a spacer and stabilizer for rollers 280. Shaft 284 may not be needed if pins are secured sufficiently to housing 294. Or, shaft 284 can be provided but not contact or loosely contact rollers 280. To the extent that shaft 284 does contact rollers 280, the shaft may be self-lubricating. Shaft 284 and/or rollers 280 can be slotted or grooved to reduce the amount of surface contact between them.

Rollers 280 are housed inside housing 294. Housing 294 in the illustrated embodiment includes a base 292 and a cover 296 that snap-fits or press-fits onto base 292. Base 292 and cover 296 of housing 294 cooperate to separate rollers 280 from one another via pins 282 and hold each roller 280 in a rotatably fixed manner. When inserted over shaft 284, shaft 284 also acts to keep rollers 280 separated.

In one embodiment, radially extending slots are provided in base 292 and cover 296. Pins 282 and rollers 280 can move radially in and out relative to base 292 and cover 296. Here like above, housing 294 and rollers 280 are dimensioned so that when shaft 284 is not present, the tube 76 pushes rollers 280 inward towards each other. That is, without a counteracting force, the tube 76 elastically pushes rollers 280 inwards. Shaft 284 includes a coned end 288. When inserted between rollers 280, coned end 288 gradually pushes rollers 280 apart radially from one another, so that shaft 284 eventually completely compresses the tube 76 between the bearing surface of the casing and the rollers 280. As before, such compression completely occludes the tube 76 at each of the rollers 280.

In an alternative embodiment, base 292 and cover 296 are not slotted so that pins 282 and rollers 280 cannot move radially in and out relative to base 292 and cover 296. Here unlike above, housing 294 and rollers 280 are dimensioned so that when shaft 284 is not present, the tube 76 does not push rollers 280 inward towards each other. Shaft 284 may therefore be eliminated if base 292 and cover 296 of housing 294 and pins 282 are robust enough. Here, the location of the pins 282 completely compresses the tube 76 between the bearing surface of the casing and the rollers 280, so that the tube 76 is occluded completely at each of the rollers 280.

As seen in FIGS. 9B and 9C, when cassette 50 is placed into unit 60, pins 282 fit into grooves 262 of groove plate 260. Groove plate 260 is coupled to shaft 272 of motor 270, e.g., via a set screw 264 or other method known to those of the art. While groove plate 260 is shown coupled directly to shaft 272 of motor 270, a belt and pulley or ratio gear assembly may be used alternatively. In the illustrated embodiment, friction between plate 260 and shaft 284 is reduced by placing bearings, such as ball bearings 274, between shaft 284 and plate 260.

As seen in FIGS. 9B, 9C and 9D, grooves 262 are separated by angled stops 266. Three stops 266 are provided, one for each pin 282. The stops 266 are spaced apart the same as pins 282, e.g., at 120 degrees, about centerline 286. Angled stops 266 each include a substantially vertical face and an angled face 268. Angled faces 268 enable self-alignment. When pins 282 are forced against angled faces 268, groove plate 260 and/or pins 282 rotate until pins 282 bottom-out against the grooves 262 of plate 260. At that point, cassette 50, pins 282 and rollers 280 are locked vertically into unit 60 and plate 260. Motor 270 and plate 260 spin with respect to pins 282 and rollers 280 until the vertical faces of stops 266 abut pins 282. At that point, pins are locked against stops 266 of plate 260, and motor 270 can thereafter positively drive pins 282, rollers 280 and fluid through the tube 76. As plate 260 spins rollers 280 about centerline 286, the friction between rollers 280 and tube 76 causes rollers 280 to rotate individually about pins 282 (or rollers 280 and pins 282 rotate integrally together within housing 294). The embodiment of FIGS. 9B to 9D is unidirectional, however, a bidirectional motor can be employed if needed to help release jamming.

Referring now to FIGS. 9E and 9F, alternative pumps 290 and 310 are illustrated. Pumps 290 and 310 are both positive drive pumps, like pump 250. Also, like pump 250, pumps 290 and 310 include a variable number of pump rollers 280, e.g., three. Rollers 280, as above are made of any suitable metal, plastic, composite or other material and in one embodiment are made of a fiber reinforced material. Rollers 280 are located inside a loop formed by the deformable tube 76 (illustrated in FIGS. 4, 7 and 8). Each roller 280 rotates about a pin 302 (pump 290), 322 (pump 310). Pins 302, 322 may be made of a self-lubricating material and function also as a cylindrical bearing. Alternatively, roller or ball bearings (not illustrated) may be placed between rollers 280 and pins 302, 322 to reduce friction. Further alternatively, pins 302, 322 may be integral to and rotate with rollers 280. In one embodiment, rollers 280 are identical to one another as are the pins. Rollers 280 and pins 302, 322 are spaced apart evenly, e.g., at 120 degrees, about centerline 286.

Pumps 290 and 310 also include shaft 284, which does not drive rollers 280. Instead, shaft 284 acts as a spacer and stabilizer for rollers 280. Shaft 284 may not be needed if pins 302, 322 are secured sufficiently within respective roller and pin assemblies 300, 320 of pumps 290, 310. Or, shaft 284 can be provided but not contact or loosely contact rollers 280. To the extent that shaft 284 does contact rollers 280, the shaft may be self-lubricating. Shaft 284 and/or rollers 280 can be slotted or grooved to reduce the amount of surface contact between them.

As mentioned, rollers 280 are housed within respective roller and pin assemblies 300, 320 of pumps 290, 310. Assemblies 300, 320 replace multiple piece housing 294 of pump 250. Assemblies 300, 320 cooperate with, e.g., fit inside an aperture defined by, pump casing 78. Assembly 300 in FIG. 9E includes a top 304 and a bottom 306. Top 304 and bottom 306 of assembly 300 cooperate to separate rollers 280 from one another via pins 302 and hold each roller 280 in a rotatably fixed manner. When inserted over shaft 284, shaft 284 also acts to keep rollers 280 separated. Likewise, assembly 320 in FIG. 9F includes a top 324 and a bottom 326. Top 324 and bottom 326 of assembly 320 cooperate to separate rollers 280 from one another via pins 322 and hold each roller 280 in a rotatably fixed manner.

In one embodiment, radially extending slots are provided in top 304, 324 and bottom 306, 326. Pins 302, 322 and rollers 280 can move radially in and out relative to top 304, 324 and bottom 306, 326. Top 304, 324, bottom 306, 326 and rollers 280 are dimensioned so that when shaft 284 is not present, tube 76 pushes rollers 280 inward towards each other. That is, without a counteracting force, tube 76 elastically pushes rollers 280 inwards. Shaft 284 again includes a coned end 288. When inserted between rollers 280, coned end 288 gradually pushes rollers 280 apart radially from one another, so that shaft 284 eventually completely compresses tube 76 between the bearing surface of casing 78 and rollers 280. As before, such compression completely occludes tube 76 at each of the rollers 280.

In an alternative embodiment, top 304, 324, bottom 306, 326 are not slotted so that pins 302, 322 and rollers 280 cannot move radially in and out relative to top 304, 324 and bottom 306, 326. Here, top 304, 324, bottom 306, 326 and rollers 280 are dimensioned so that when shaft 284 is not present, the tube 76 does not push rollers 280 inward towards each other. Shaft 284 may therefore be eliminated if top 304, 324, bottom 306, 326 and pins 302, 322 are robust enough. The location of pins 302, 322 again causes the complete compression of tube 76 between the bearing surface of the casing 78 and the rollers 280, so that tube 76 is occluded completely at each of the rollers 280.

Unlike pump 250, pins 302, 322 of assemblies 300, 320 of pumps 290, 310 do not extend into the respective drive plates 305, 325 of pumps 290, 310. Pins 302, 322 instead hold rollers 280 rotatably within respective assemblies 300, 320. The direct drive interface instead takes place between mating teeth 308, 328 of drive plates 305, 325 and bottoms 306, 326 of assemblies 300, 320.

Drive plates 305, 325 serve a similar purpose as groove plate 260 of pump 250. Drive plates 305, 325 are coupled to shaft 272 of motor 270, e.g., via a set screw 264 or other method known to those of the art. While drive plates 305, 325 is shown coupled directly to shaft 272 of motor 270, a belt and pulley or ratio gear assembly may be used alternatively. As above, friction between drive plates 305, 325 and shaft 284 may be reduced by placing bearings, such as ball bearings 274, between shaft 284 and drive plates 305, 325.

For pump 290, when cassette 50 is placed into unit 60, teeth 308 of drive plate 305 mate with teeth 308 of bottom 306 of assembly 300. In the illustrated embodiment (FIG. 9E), teeth 308 are matching sharp, e.g., triangular shaped, with sides angled at about 45 degrees. The triangular shaped teeth provide for a fast-loading and self-adjusting interface between drive plate 305 and assembly 300. When cassette 50 and rollers 280 are locked vertically into unit 60, mating teeth 308 also lock together in a self-aligning manner. Motor 270 can thereafter positively move drive plate 305, assembly 300, rollers 280 and fluid through tube 76. As assembly 300 spins the rollers 280 about centerline 286, the friction between rollers 280 and tube 76 causes rollers 280 to rotate individually about pins 302 (or rollers 280 and pins 302 rotate integrally together within assembly 300). Motor 270 can be a bidirectional motor and positively drive rollers 280 about centerline 286 in two directions.

For pump 310, when cassette 50 is placed into unit 60, teeth 328 of drive plate 325 mate with teeth 328 of bottom 326 of assembly 320 and self-adjust until locking together. In the illustrated embodiment (FIG. 9F), teeth 328 are rounded and U-shaped, e.g., including vertical edges with rounded or circular ends.

Motor 270 can thereafter positively move drive plate 325, assembly 320, rollers 280 and fluid through tube 76. As assembly 320 spins the rollers 280 about centerline 286, the friction between rollers 280 and the tube causes rollers 280 to rotate individually about pins 322 (or rollers 280 and pins 302 rotate integrally together within assembly 320). Motor 270 can be a bidirectional motor and positively drive rollers 280 about centerline 286 in two directions.

The U-shaped teeth 328 also provide for a relatively quick-loading and self-adjusting interface between drive plate 325 and assembly 320. Teeth 328 have vertically interfacing drive faces, producing a more lateral push than teeth 308, and do not create vertical force vectors, reducing friction and PM. The U-shaped engagement is more positive and accurate than the angled teeth 308 of pump 290. This reduces the amount of force needed to drive rollers 308 and torque needed from motor 270.

FIG. 9G illustrates an exploded view of assembly 320 of pump 310. As seen above for pump 250, housing 294 requires two pieces 292 and 296 that snap-fit together. This configuration requires more precise tooling, manufacturing and assembly. Top 324 and bottom 326, on the other hand, are formed together as a body 330 of assembly 320. Top 324 and bottom 326 of body 330 each define matching slots 332. Each slot receives a pin 322 coupled integrally or rotatably to a roller 280. Slots 332 define indents or detents so that pins 322 can snap into slots 332 for easy formation of assembly 320. Slots 322 space rollers 280 apart as desired. Assembly 300 of pump 290 is similar to assembly 320 of pump 310 except for teeth 308 versus teeth 322.

### Peristaltic Pump Tubing Materials

It should be appreciated that tubing 76, which: (i) is crimped between rollers 80 and surface 74, (ii) is rolled upon by rollers 80 and (ii) thereafter expanded, is subject to a fair amount of stress. It should also be appreciated that the performance of such tubing is important to the medical fluid therapy for accuracy, reliability and durability reasons.

One major aspect of the present invention is to provide a new tubing for peristaltic pump applications. The new tubing can be used in any of the circuits or tubing lines shown in FIG. 1, such as through tubes 32, 68, 20, 54, 28 and 12. In particular, the tubing of the present invention is well-suited and used for tube 76, which is contacted and compressed between rollers 80 and surface 74. For that reason, the remainder of the description refers to tubing 76, however, it should be appreciated that the tubing can be used for any of the tubes, circuits or circuit segments described above.

The improved peristaltic pumping tubing offers better quality and a competitive or lower cost alternative to the known peristaltic pump silicone tubing. The improved tubing of the present invention is made from any one or more or all of the following materials: high quality silicone, silicone blend, ethylene propylene diene monomer (“EPDM”), polyurethane (“PU”), polyvinylchloride (“PVC”), ultra-high molecular weight PVC (“UHMWPVC”), styrene block copolymer, metallocene-catalyzed ultra-low density polyethylene (“m-ULDPE”) and polytetrafluoroethylene (“PTFE”) and any combination thereof. The above-listed materials alone or in combination exhibit desirable properties with respect to standard silicone when subjected to the stresses of peristaltic pumping as is shown in Tables 2 and 3 below.

Tubing 76 is assembled to disposable cassette 50 via bulkhead ports 96 and 98, e.g., by mechanical attachment. The tubing is alternatively extruded from such cassette, molded to such cassette, extrusion molded to cassette 50, bonded to cassette 50, radio frequency (“RF”) sealed or heat sealed to cassette 50, laser welded or attached to cassette 50 or otherwise attached via any combination of the above. Solvent bonding is particularly desirable because it provides a cost effective approach for manufacturing the cassettes on a large scale.

In particular, PVC, UHMWPVC and PU are readily solvent bonded to cassette 50, which in one embodiment is acrylic, but is alternatively polycarbonate, acrylonitrile butadiene styrene (“ABS”), PVC and UHMWPVC. The cassette 50 and tubing 76 are sterilized via either EtO or radiation. EPDM and low PM silicone can both be friction fitted to the cassette. Here, EtO sterilization fluid penetrates through the low PM silicone and EPDM tubing wall thickness to establish sterility at the tubing/cassette interface. PU can be either solvent bonded or friction fitted to cassette ports.

Cassette 50 includes rigid walls or portions as seen in FIGS. 4 to 7. Those rigid portions can be made of the same material as tubing 76 or made of a different material. The tubing and/or cassette attached thereto can then be sterilized by any suitable process, such as via an ethylene oxide (“EtO”) wash or via radiation, such as gamma radiation or electron beam radiation.

It is important that while tubing 76 is shelved or stored before use that the tubing and associated cassettes meet and retain the necessary functionality, sterility and integrity. The above-described tubing materials are well-suited for such application because they offer a desirable balance between properties, such as compression set, tubing flexibility as well as uniformity of diameter and consistency of wall thickness. Those properties each, to at least a certain extent, effect fluid volume accuracy and tube spallation. The properties of the tubing materials used for tube 76 of the present invention cause or help to cause the resulting fluid volume to be accurate. The properties also minimize tube spallation, rendering low particulate matters (“PM”).

Fluid volume accuracy and low PM are critical factors when delivering a premixed dialysate to the patient as described above in connection with FIG. 1. Additionally, in certain therapies, such as in peritoneal dialysis, some solution formulations cannot be stored in mixed form for extended periods of time. One such situation occurs when one solution has a low pH and second solution has a high pH. In such cases, the separate solutions have to be mixed at or near the point and/or time of use. Those solutions are mixed, e.g., in a one-to-one ratio to yield an overall solution having a pH at a desirable physiologic level. Fluid volume accuracy and low PM are two critical factors when admixing such solutions together.

Moreover, for any type of renal therapy including any of the types described above, it is important to accurately infuse and drain fluid to and from the patient 18 and to properly balance the body fluids. Not only does the peristaltic pump tubing of the present invention achieve accurate fluid volume over an entire therapy, it does so at conditions of extreme pH, e.g., for admixing, and also at extreme head heights, such as +0.5 m to −0.5 m, and vice versa. Moreover, the tubing provides for accurate fluid volumes over a wide temperature range, such as from 4° C. to 40° C. At those conditions, the tubing and cassette accurately admix solution components, infuse a properly mixed solution to the patient 18, as well as drain spent fluid from the patient. The tubing materials at the same time will render low spallation over operating periods of up to twenty-four hours, which will result in low PM to patient 18.

Table 1 below shows various properties or characteristics of tubing materials suitable for use in the peristaltic application of the present invention. The list is illustrative and not exhaustive. Briefly summarizing some of the important features of Table 1, it should be noted that the tubing of the present invention has a Shore A Hardness in a range of 50 to 85. The tubing is shown to have a compression set in a range of 30% to 65% for a tubing temperature of 73° C. by 22 hours. The tubing is also shown to have a tear resistance in a range of 110 to 480 in-lb per inch. SIPLA A/S (ASICOMO), Silicone—Peroxide Cured is listed first as a control material for comparison with the remainder of the tubing materials of the present invention.

TABLE 1

| | | | | | | IMPACT | |
|---|---|---|---|---|---|---|---|
| | | | TENSILE | TENSILE STRESS | | REBOUND | COMPRESSION |
| | | TENSILE | ELONGATION | @ 300% | TEAR | RESILIENCE | SET (73° F./ |
| | HARDNESS in | STRENGTH in | AT BREAK | elongation | RESISTANCE in | ELASTICITY | 22 hrs) |
| | Shore A | psi | in percent | in psi | lbf/in | in percent | in percent |
| CONTROL | 60 | 1,450 | 500 | | 126 | 46 | 30 |
| SIPLA A/S | | | | | | | |
| (ASICOMO) | | | | | | | |
| Silicone - Peroxide | | | | | | | |
| Cured | | | | | | | |
| SAINT GOBAIN | 65 | 700 | 400 | 375 | 110 | | 36 |
| PharmaPure | | | | | | | |
| EPDM Co-extrude | | | | | | | |
| SAINT GOBAIN | 56 | 1,550 | 380 | 900 | 122 | | 64 |
| Tygon LFL | | | | | | | |
| UHMW PVC | | | | | | | |
| DOW | 85 | 4,210 | 650 | 890 | 420 | | 30 |
| Pellethane 2363- | | | | | | | |
| 80Ae | | | | | | | |
| Polyurethane | | | | | | | |
| SAINT GOBAIN | 64 | 1,050 | 375 | 400 | 128 | | 32 |
| PharMed | | | | | | | |
| EPDM & | | | | | | | |
| Polypropylene | | | | | | | |
| SAINT GOBAIN | 69 | 800 | | | 174 | | |
| MPF-500 | | | | | | | |
| EPDM, Polyolefin | | | | | | | |
| & Viton | | | | | | | |
| SAINT GOBAIN | 66 | 2,000 | 350 | 1,100 | 165 | | 53 |
| Tygon S-50-HL | | | | | | | |
| PVC | | | | | | | |
| TEKNOR APEX | 74 | 2,235 | 360 | 700 | | | 16 |
| 03-U0473A-68NT | | | | | | | |
| UHMW PVC | | | | | | | |
| POLYONE | 61 | 830 | 620 | 550 | 200 | 50 | 20 |
| Synprene RT- | | | | | | | |
| 3860 M | | | | | | | |
| Styrene Block | | | | | | | |
| Copolymer | | | | | | | |
| SAINT GOBAIN | 50 | 1,300 | 600 | 230 | 210 | 48 | 14 |
| GE SE4524U | | | | | | | |
| Silicone - Peroxide | | | | | | | |
| Cured | | | | | | | |
| NEW AGE | 50 | 1,136 | 1,284 | | 202 | | |
| OptiFlex SR-110 | | | | | | | |
| Styrene Block | | | | | | | |
| Copolymer | | | | | | | |
| NEW AGE | 75 | 2,165 | 1,540 | | 476 | | |
| OptiFlex PEBA | | | | | | | |
| Styrene Block | | | | | | | |
| Copolymer | | | | | | | |
| Dupont-Dow | 77 | 9.8 | 920 | | | | |
| Engage m-ULDPE | | Mpa | | | | | |
| Engage 8452 | | | | | | | |

With respect to peristaltic pumping, the above materials will hold their shape and dimensioning well over time, and in the presence of sterilizing agents. That is, aging and sterilization will have a negligible or otherwise acceptable impact with respect to tubing shape, tubing length and wall thickness. Furthermore, the tubing material of the present invention has a surface friction coefficient suitable for receiving the rollers 80 of the peristaltic pump and for enabling such rollers to operate frictionally as described above. The tubing during such operation exhibits an acceptable impact and tear resistance over the entire course of therapy.

The tubing materials described above for peristaltic pumping exhibit excellent biocompatibility with the fluids used, as well as low toxicity and extractives. It is believed that EPDM and UHMWPVC in particular have a substantially lesser percent PM, e.g., two percent, than the level exhibited by known silicone tubing. EPDM also is particularly compatible with low pH solutions, such as those solutions used in admixing applications. Furthermore, PU tubing exhibits good abrasion resistance for peristaltic pumping.

Each of the tubing materials described above is easily extruded relative to silicone. Such extrusions yield consistent tubing diameter and wall thickness, which increases fluid volume accuracy. The materials balance compression set with flexibility, making the tubing relatively easy to compress and to occlude properly, leading to accurate volume pumping. The consistent tubing dimensions and material properties also yield a low percentage change in flow output over an entire pumping cycle. The ability to withstand extreme pH also makes the materials well-suited for PD, hemodialysis, hemofiltration and hemodiafiltration applications either in center or at home. It has also been found that the tubing materials achieve low PM and high fluid volume accuracy when subjected to pump stresses for up to 12 hours at broad temperatures, extreme head heights and extreme pH levels.

Shown below are two tables, namely Tables 2 and 3, that recite the findings from a low pH study and a high pH study. Each study included three trials. Each study compared standard silicone, used typically with peristaltic pumping, to EPDM and UHMWPVC, two of the more preferred materials of the present invention. The trials spanned a total of approximately twelve hours at such high pH and head height levels, wherein the tubing was subjected to continuous forces and stresses from a peristaltic pump head.

The tables show that the tubing materials of the present invention offer many advantages compared to the known silicone tubing. Besides being at least comparable in cost with respect to standard silicone, the tubing materials tested showed lower PM at pH levels between 1.8 and 9.2. The tubing materials exhibited high fluid volume accuracy between temperatures of 4° C. and 40° C. The materials exhibited high fluid volume accuracy at extreme head heights of ±0.5 m, rendering a total head height change of 1 m.

The tables show that EPDM and UHMWPVC perform better than known silicone with respect to fluid volume accuracy at 30 minutes, and also at 250 minutes, of peristaltic pumping. The last time entry of each trial shows the accuracy when the head height is changed immediately from one extreme to another, e.g., from +0.5 m to −0.5 m, and vice versa. The test results show that EPDM and UHMWPVC performed better than known silicone after the head height reversal. The results were consistent at the low pH of 2 in Table 1 as well as a high pH of 9 in Table 2. Indeed, both the EPDM and UHMV-PVC exhibited less than a ten percent change in flow over time despite the wide range of pH level, source head height and user temperature. The three trials of each table were conducted on the same pieces of tubing and show that the results do not diminish appreciably over an entire twelve hour therapy.

Fluid volume accuracy as shown below is based on the amount of fluid infused into the patient 18 and the amount fluid pulled out of the patient 18. The accuracy is at least ninety percent, preferably at least ninety-five percent and most preferably at least ninety-nine percent. That is, for any fill and drain cycle, the amount of fluid infused into the patient and the amount of fluid pulled from the patient are within ninety percent of one another, preferably within ninety-five percent of one another and most preferably within ninety-nine percent of one another. The accuracy data shown below reflects the above-defined type of fluid volume accuracy, that is, amount of fluid infused into the patient 18 and the amount of fluid pulled out of the patient 18 over one therapy cycle.

In one embodiment, the fluid volume accuracy achieved by the tubing of the present invention achieves the same level of accuracy (e.g., at least ninety percent) when evaluated in other ways. In one additional way, the volume drained from the patient and the initial treatment volume pulled from a supply bag and delivered to the patient are at least ninety percent the same, preferably at least ninety-five percent the same and most preferably at least ninety-nine percent the same. Typically, a patient about to receive PD treatment has a “last fill” volume of fluid residing in the patient's peritoneum. That “last fill” volume is removed at the beginning of therapy, after which the first volume of fresh dialysate is delivered to the patient. This second type of accuracy refers to those two fluid volumes or amounts.

In another embodiment, the total volume of fluid delivered to the patient 18 and the total volume removed from the patient 18 over the entire therapy, including multiple cycles and occurring over, e.g., nine hours, are at least ninety percent the same, preferably at least ninety-five percent the same and most preferably at least ninety-nine percent the same. It is believed that the characteristics of the tubing will change over multiple cycles and multiple hours, so that less fluid will be delivered to the patient 18 in a later cycle than in an earlier cycle. Less fluid will correspondingly be removed from the patient 18 in a later cycle than in an earlier cycle due to the changing characteristics of the tubing. The change in volume delivered is roughly the same for fluid infused versus fluid removed, keeping the overall net balance of fluid delivered within the accuracy limits described above.

TABLE 2
Low pH Study

| Summary of Pump | Trial #1 | Trial #2 | Trial #3 |
|---|---|---|---|
| Tubing Accuracy at | (40 C., +0.5 m) | (40 C., −0.5 m) | (4 C., −0.5 m) |
| different environments | Low pH = 2.0 | Low pH = 2.0 | Low pH = 2.0 |
| Volume Accuracy | % Accuracy | % Accuracy | % Accuracy |
| Silicone | | | |
| (hr.:min.) | | | |
| 0 | 100 | 100 | 100 |
| 30 | 99 | 96 | 90 |
| 4:00 | 97 | 90 | 83 |
| 4:30* | 81 | 112 | 112 |
| | (+0.5 m to −0.5 m) | (−0.5 m to +0.5 m) | (−0.5 m to +0.5 m) |
| EPDM | | | |
| 0 | 100 | 100 | 100 |
| 30 | 100 | 96 | 97 |
| 4:00 | 97 | 92 | 95 |
| 4:30* | 90.45 | 99.51 | 103.01 |
| | (+0.5 m to −0.5 m) | (−0.5 m to +0.5 m) | (−0.5 m to +0.5 m) |
| UHMWPVC | | | |
| 0 | 100 | 100 | 100 |
| 30 | 100 | 99.64 | 97.63 |
| 4:00 | 97.7 | 95.62 | 97.56 |
| 4:30* | 90.97 | 101.46 | 102.01 |
| | (+0.5 m to −0.5 m) | (−0.5 m to +0.5 m) | (−0.5 m to +0.5 m) |

*Head height converted at that instant from first listed height to second listed height.

TABLE 3
High pH Study

| Summary of Pump Tubing | Trial #1 | Trial #2 | Trial #3 |
|---|---|---|---|
| Accuracy at Different | (40 C., +0.5 m) | (40 C., −0.5 m) | (4 C., −0.5 m) |
| User Environments | High pH = 9.0 | High pH = 9.0 | High pH = 9.0 |
| Volume Accuracy | % Accuracy | % Accuracy | % Accuracy |
| Silicone | | | |
| (hr.:min.) | | | |
| 0 | 100 | 100 | 100 |
| 30 | 99 | 96 | 91 |
| 4:00 | 97 | 90 | 87 |
| 4:15* | 80 | 110 | 118 |
| | (+0.5 m to −0.5 m) | (−0.5 m to +0.5 m) | (−0.5 m to +0.5 m) |
| EPDM | | | |
| 0 | 100 | 100 | 100 |
| 30 | 99 | 97 | 98 |
| 4:00 | 97 | 92 | 95 |
| 4:15* | 91 | 100 | 105 |
| | (+0.5 m to −0.5 m) | (−0.5 m to +0.5 m) | (−0.5 m to +0.5 m) |
| UHMWPVC | | | |
| 0 | 100 | 100 | 100 |
| 30 | 99 | 98 | 100 |
| 4:00 | 97 | 91 | 98 |
| 4:15* | 90 | 98 | 103 |
| | (+0.5 m to −0.5 m) | (+0.5 m to +0.5 m) | (−0.5 m to +0.5 m) |

*Head height converted at that instant from first listed height to second listed height.

### Cassette Improvements

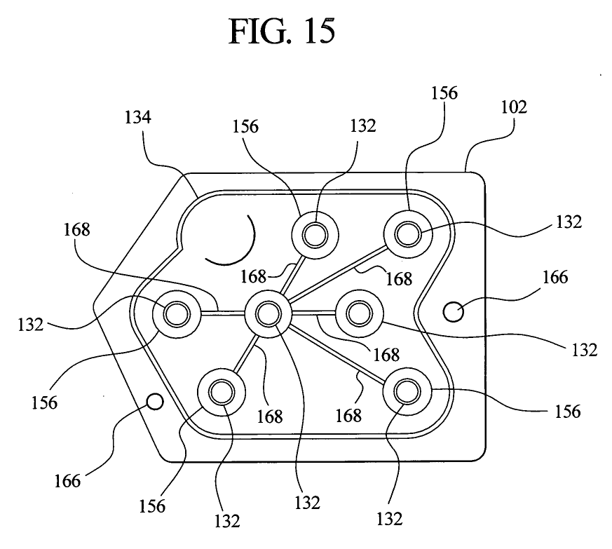

Referring now to FIGS. 6, 7 and 10 to 15, various improvements to the cassette 50, flexible membrane 102, and the attachment of membrane 102 to cassette 50 are illustrated. FIG. 6 illustrates cassette 50 with membrane 102 removed. FIG. 7 illustrates cassette 50 with membrane 102 installed. FIGS. 6 and 7 both illustrate a membrane shape 134, which is defined both by cassette 50 (groove) and membrane 102. As discussed above, cassette 50 and membrane 102 are in one preferred embodiment sonically welded together and are therefore made of materials that are compatible for such hermetic sealing. Alternatively, membrane 102 can be mechanically sealed to cassette 50. In that case, a mechanical force is applied along the perimeter 134 of membrane 102, at which in one embodiment a sealing flange or projection is provided.

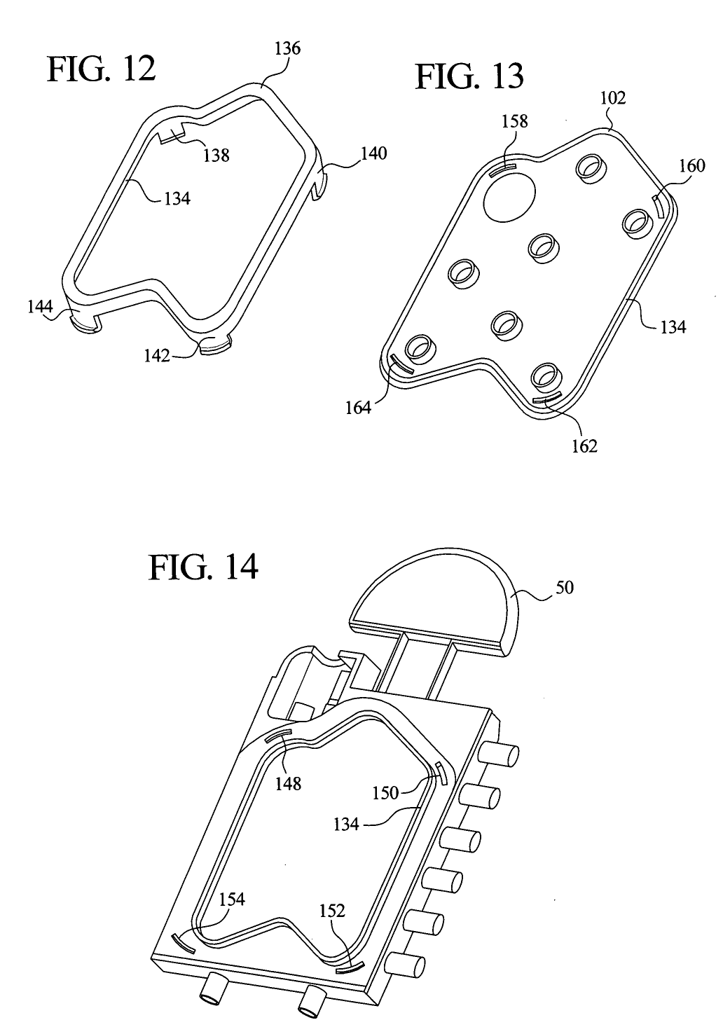

FIGS. 12 to 14 illustrate one improved apparatus for mechanically sealing membrane 102 to cassette 50. In mechanical attachment, a silicon material may be used for membrane 102 as opposed to the PVC membrane described above. Silicon in general is more flexible than PVC. FIG. 12 illustrates an improved holding ring 136, which is made in the shape 134, common to both membrane 102 and the perimeter on cassette 50. Improved holding ring 136 includes or defines locking members 138, 140, 142 and 144. Locking members 138, 140, 142 and 144 snap-fit or press-fit respectively into apertures 148, 150, 152 and 154, defined by cassette 50. Alternative membrane 102 in turn defines apertures 158, 160, 162 and 164 through which members 138, 140, 142 and 144 are respectively inserted to lock membrane 102 between holding ring 136 and cassette 50.

Improved holding ring 136, corresponding membrane 102 and cassette 50 enable membrane 102 to be fixedly, if not sealingly, held in place, while cassette 50 is being transported to actuator unit 60 for use. In that way, cassette 50 can be mechanically preassembled so as not to require the operator to align membrane 102 with cassette 50 and a sealing ring when installing cassette 50 into actuator 60 of system 10.

The assembly provided by improved holding ring 136, membrane 102 and cassette 50 is more robust than previous cassettes and is better suited for handling, shipping and therapy installation. Further, the assembled structure is tamper evident. In one embodiment, members 138 to 144 lock permanently in place in cassette 50, so that the rigid plastic pieces 136 or 50 would have to be modified or tampered with to pull ring 136 apart from cassette 50.

In one embodiment, improved holding ring 136 is carbon fiber reinforced polycarbonate or carbon fiber filled polycarbonate. The members 138 to 144 can be narrowed in profile to help guide ring 136 in place during assembly. The snap-fit assembly ensures that the position of ring 136 relative to the membrane 102 and the corresponding groove 134 in cassette 50 are aligned properly and maintained that way during shipping and handling.

While four locking members are illustrated, any suitable number of snap-fitting members and corresponding apertures can be provided. Further, the periphery of membrane 102 can be shaped and sized to receive protrusions extending downward from ring 136 and upward from cassette body 50. When ring 136 and body 50 are snap-fitted together, the protrusions form a pinch point along the periphery of membrane 102 to enhance the seal. The perimeter 134 of membrane 102 can also be contoured or shaped to maximize the sealed surface area between membrane 102 and rigid pieces 136 and 50.

As discussed above, membrane 102 can be mechanically, chemically or sonically coupled and sealed to cassette 50. A PVC membrane ultrasonically welded to cassette 50 generates a hermetic seal that is desirable with respect to the mechanical seal. The hermetic seal will guarantee that no fluid leaks exist when cassette 50 is produced. Regardless of the type of seal between membrane 102 and cassette 50, it is desirable to widen the sealing ribs 176 on cassette 50, seen best in FIG. 6, as much as possible. Sealing ribs 176 help to define the cassette flow paths, such as flow path 114 described above in connection with FIG. 6. Sealing ribs 176 seal against membrane 102 and surface 108 of actuator 60 shown in FIG. 3 when cassette 50 is installed inside actuator unit 60 to form assembly 50, as shown in FIG. 2. Cassette 50 is mechanically press-fitted against surface 108, which compresses membrane 102 against ribs 176 to complete the fluid flow paths located on the underside of cassette 50 when installed.

For proper sealing, it is desired to make the sealing ribs 176 as wide as possible. For example, the width of ribs 176 can be increased from 0.010 inch (0.254 mm) to a range from about 0.015 inch to about 0.030 inch (0.038 mm to about 0.076 mm). The widened sealing rib provides a number of advantages. A first advantage listed above is for improved mechanical or chemical sealing. A second advantage occurs when membrane 102 is ultrasonically sealed to and along ribs 176, where the widened rib is better able to withstand the heat generated during such process. Third, the process for making cassette 50 in one embodiment is an injection molding process. That process yields more accurate and consistent parts when ultra-thin members, such as existing ribs, are widened to form thicker ribs 176.

FIG. 6 illustrates another improvement in the cassette 50 of the present invention. Here, ribs 176a and 176b (shown in phantom) illustrate two examples of where the previous ribs, which were curved, are now straightened out. It is easier to seal straight sides to membrane 102 than it is to seal the curved ribs that existed previously. The straightened out or flattened out ribs 176a and 176b are advantageous in multiple situations where membrane 102 is mechanically, chemically or sonically sealed to ribs 176a and 176b. It is therefore expressly contemplated in the present invention to smooth out or flatten out any sharp edges or curves of any enclosed loop defined by any of the ribs 176 of cassette 50, where possible and practical.

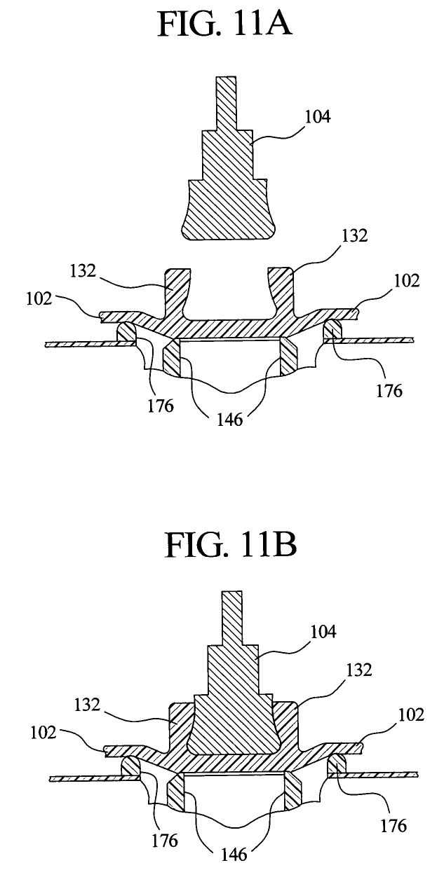

FIGS. 11A, 11 and 15 illustrate an improved membrane 102 for ultrasonically sealing the membrane to cassette 50. One improvement is illustrated by FIGS. 11A and 11B. Those figures show a valve actuator 104 in a disengaged and engaged position, respectively, with respect to a coupler 132 of membrane 102. Couplers 132 specific to valves 26, 44, 36, 30 and 42 are also shown for reference in FIGS. 7 and 10. There is a valve actuator 104 for each valve 26, 44, 36, 30 and 42 and an engagement flange 132 is provided for each one of those valves. Although too difficult to see in FIGS. 4 and 6, cassette 50 defines valves 26, 44, 36, 30 and 42 via valve tubular walls 146 shown in FIGS. 11A and 11B. To close one of the valves, actuator 104 engages its respective coupler 132 and pushes the coupler and membrane 102 against the edge of tubular wall 146. To open one of the valves, actuator 104, while engaged to its respective coupler 132, is pulled away from wall 146, pulling coupler 132 and membrane 102 away from the wall and enabling fluid to enter the chamber defined by cassette 50 and ribs 176.

To ultrasonically seal membrane 102 to cassette 50, a suitable ultrasonic material, such as PVC, is used. PVC is more rigid than the silicon material used for mechanical sealing. Accordingly, it is desirable to widen the diameter or width struck by ribs 176, so that the distance between ribs 176 and walls 146 is increased. The increased distance lessens the force needed to be exerted by actuator 104 on coupler 132 when actuator 104 is pulled away from the valve. In essence, the valve will be easier to open and close by increasing the distance between ribs 176 and walls 146 as much as possible. This is desirable for the less flexible PVC material. Accordingly, the diameter or width of the fluid path formed by ribs 176 is increased in the cassette 50 of the present invention a suitable distance, such as 80 thousandths of an inch.

As seen in FIG. 15, each coupler 132 includes an outer diameter 156 that substantially matches the diameter or width of rib 176. Outer diameters 156 of engagement couplers 132 are therefore increased to match the increased diameter of sealing or width of ribs 176.

FIG. 15 also illustrates that membrane 102 includes additional peripheral material (compare with membrane in FIG. 7) that extends outside of shape 134 discussed above. In one embodiment, the corresponding periphery of shape 134 of cassette 50 is made to include or define a protrusion rather than the existing groove. The protrusion on cassette 50 and the increased size of membrane 102 in FIG. 15 aids in sonically sealing membrane 102 to cassette 50. That is, the additional material of membrane 102 in FIG. 15 makes the membrane area larger than the mating rigid portion of cassette 50, which is desirable for welding. Furthermore, the more simplistic five-sided shape shown of membrane 102 in FIG. 15 is more likely to be consistently and accurately injection molded than is the more intricate shape 134. The larger area of membrane 102 compensates for warping of the membrane during the injection molding process, again, leading to a more robust welding process.

Indexing holes 166 are also be defined in the outer flange area of membrane 102 in FIG. 15 to help align membrane 102 and cassette 50 for welding. Although not illustrated, one or more injection molding melt-domes may be provided and project transversely from membrane 102. Flow leaders 168 are also provided in one embodiment, which extend from the injection molding gate outward to each of the couplers 132, which require significantly more material than the flat portion of membrane 102. Flow leaders 168 enable the relatively large couplers 132 to be filled faster and more consistently, reducing manufacturing inaccuracies. Flow leaders 168 can be used additionally as an indexing device during sub-assembly.

### Apparatus and Method for Regulating Pump Pressure

Referring now to FIGS. 1, 3, 4, 5, 16 and 17, one embodiment for an apparatus and associated method for measuring and compensating for pressure due to patient head height is illustrated. Cassette 50 includes a fluid path and valve chamber portion 48 that is operable with motor/valve actuator unit 60. The path and chamber portion 48 of cassette 50 is mechanically, sonically or chemically coupled to flexible membrane 102 located along a bottom surface thereof as described above. Flexible membrane 102 can be made of any of the above-described polymers or other suitable polymer, and in one preferred embodiment is PVC as described above. Flexible membrane 102 provides a flexible surface to operate with valve actuators 104 as seen in FIGS. 3, 11A and 11B. Valve actuators 104 push against flexible membrane 102 to enable fluid flowing through tubes 28, 54, 20, 68, 32 and 12 to selectively enter or not enter cassette 50.

In FIG. 4, membrane 102 is removed to illustrate that cassette 50 includes rigid vertical walls defining, among other items, a fixed volume chamber 106, various flow paths and valve chambers 44, 26, 42, 40, 36, 34 and 30. It should be appreciated that cassette 50 can define any suitable number of volume chambers, such as chamber 106, flow paths and valve chambers, such as chambers 44, 26, 42, 40, 36, 34 and 30. For ease of illustration, however, only the above elements are numbered.

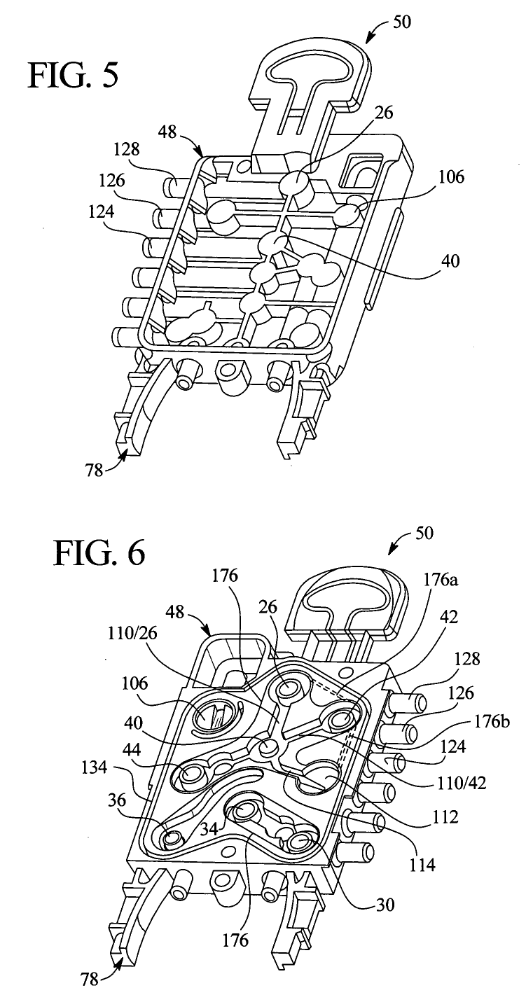

FIG. 4 shows the lower or flexible membrane side of cassette 50 that engages surface 108 of actuator unit 60 in FIG. 3. FIG. 5 shows the upper or rigid flow path side of cassette 50. The upper side of FIG. 5 faces door 70 in FIG. 2. Membrane 102 (not seen in FIG. 4) covers the bottom of valve chambers 44, 26, 42, 40, 36, 34 and 30 as well as the bottom of chamber 106, and seals to the bottom of the walls defining those structures in a similar manner as described above with the ribs of FIG. 4.

Supply tubes 28, 54 and 20 are connected fluidly with valve chambers 26, 42 and 40, respectively via bulkhead connectors 128, 126 and 124 and internal tubes or flow paths defined by cassette 50. Fluid entering valves 26, 42, 40 can selectively flow through tube 76 and chamber 106. From chamber 106, fluid flows out cassette 50, through tube 12, to patient 18.

As seen in FIG. 3, a plurality of pressure sensors 116 are housed in the motor/valve actuator unit 60. Sensors 116 are located directly beneath, and are in contact with, flexible membrane 102. Pressure sensors 116 can be any suitable pressure sensors for sensing fluid pressure fluctuations relatively or absolutely known to those of skill in the art. For example, the pressure sensors can be provided by Invensys, Model 1865-02G-KDN. The measured pressure via sensor 116 can be either relative or absolute depending upon the type of pressure sensor used.

Flexible membrane 102 contacts fluid entering chamber 106 on one side and is coupled to pressure sensor 116 on the other side. Pressure sensor 116 senses pressure of fluid within chamber 106 and outputs a signal corresponding to or indicative of an absolute or relative pressure, or a pressure change applied to the surface of pressure sensor 116 via fluid pressure and membrane 102.

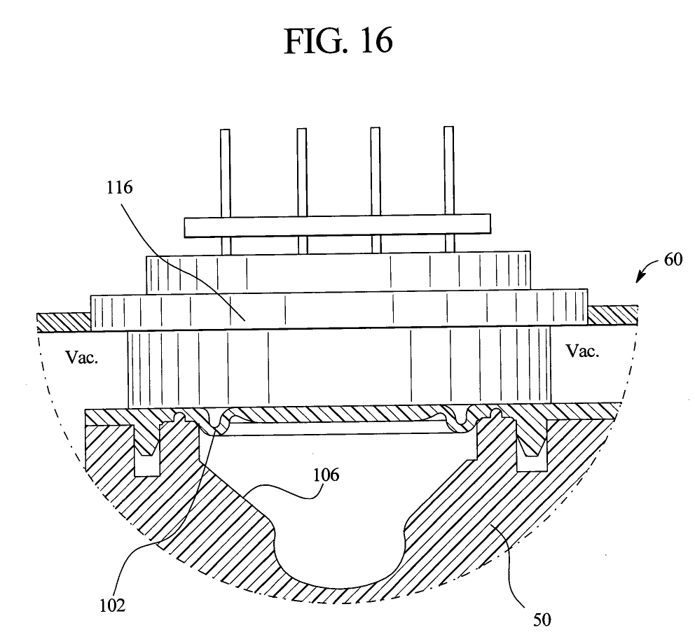

Pressure sensor 116 can be located so that it protrudes slightly into flexible membrane 102. Alternatively, as seen in FIG. 16 a slight vacuum Vac is pulled between unit 60 housing sensor 116 and the underside of membrane 102 to suction the membrane to adhere to pressure sensor 106. The vacuum Vac maintains contact between the pressure sensitive surface of pressure transducer 106 and membrane 102 at all times. The apparatus needed to pull a vacuum on membrane 102 is not illustrated, however, such apparatus resides within motor/valve actuator unit 60 in one embodiment and is known to those of skill in the art. Although not illustrated, sensor 116 may be modified to pull vacuum Vac through the sensor.

In an alternative embodiment, flexible membrane 102 is loaded onto surface 108 of motor/valve actuator unit 60 such that a gas tight seal is formed between flexible membrane 102 and pressure sensor 116 with a negligible gas volume trapped between them. That gas tight seal also enables positive and negative fluid pressures to be transmitted through flexible membrane 102 to the pressure sensing surface of pressure sensor 116.

Because the pressure sensing surface of sensor 116 does not move appreciably or distort with varying fluid pressures, the fluid pressure of dialysate or other medical fluid is directly transmitted through film 102 to the sensing surface of sensor 116. Pressure sensor 116 thereby directly measures either positive or negative pressure of the fluid inside chamber 106 of cassette 50. Because the elevation of chamber 106 is substantially the same as that of the fluid in tube 76 surrounding rollers 80 of the peristaltic pump, the pressure due to patient head height is at least approximately or substantially the same at the pump as it is at chamber 106.

Although pressure sensor 116 does not directly contact dialysate or medical fluid, the pressure sensor accurately measures the pressure of such fluid due to the thin and flexible nature of membrane 102. Sensor 116 is therefore virtually in contact with the fluid within chamber 106.

Pressure sensor 116 measures pressure due to head height, which could otherwise be calculated by the following equation:
Head pressure=ρ×g×h,
where head pressure is the calculated fluid pressure, ρ is the density of the dialysate or medical fluid, g is the acceleration due to gravity and h is the head height differential. That is, h is the difference in elevation distance between pump chamber 106 and the point 46 at which the patient fluid tube as seen in FIG. 1 enters the peritoneal cavity of patient 18. Therefore, the head height differential is also the same or approximately the same as the difference in vertical distance between point 46 at patient 18 and peristaltic pump 100 of system 10.

The above-described equation applies to fluid paths where the velocity of the fluid is zero. During operation, fluid is moving within the tubes and cassette described herein. Therefore, the reading taken by sensor 116 would likely not equal the pressure predicted by the above equation but would instead be offset from the predicted pressure by a factor accounting for pressure drop due to flow restrictions in the fluid path flowing from chamber 106 and the peristaltic pump to the patient 18. It should be appreciated, however, that the flow path defined by tubing 12 and the portion of the cassette leading to chamber 106 (see FIGS. 1, 3 and 4), if unkinked, is relatively smooth and should not produce a significant pressure drop. A certain amount of pressure drop will occur, however, due to the restricted inner diameter of the medical fluid tubing, which can be 5/32″ (4 mm) outer diameter tubing, for example. Intraperitoneal pressure (“IPP”) can also effect overall pressure measured at sensor 116, which may make the measured pressure, which is assumed to be due to head height only, different than the actual pressure due to head height. Effects of IPP are typically minimal. Regardless, sensor 116 sees the pressure differential due to patient head height and IPP, accounting for each of the above factors.

The head height adjustment sensing and correcting apparatus and method of the present invention applies to any medical fluid. In one embodiment, the apparatus and method are used to compensate for pressure due to head height of dialysate, which has a density of approximately water or one gm/cm³. Such liquid density produces a one psig pressure differential for a static head height change of 27.68 inches (0.703 m). That is, if the patient's peritoneal inlet 46 is 27.68 inches above the pump or chamber 106, and the pump is not moving fluid, the pressure in line 12 will produce a positive one psig static pressure drop at the pump, assuming IPP to be negligible. Likewise, if point 46 of the patient is located that same distance below the pump or chamber 106, the static pressure due to head height will be −1 psig, assuming IPP to be negligible.

In one embodiment of the method of the present invention, the pump is caused to pump fluid to the patient's peritoneal cavity, whereafter the pump stops momentarily, e.g., at the end of a stroke of a diaphragm pump, so that the fluid is relatively stagnant and so that the above algorithm can be applied. At that moment, pressure sensor 116 records the pressure, which is the patient's pressure due to head height according to the equation described above. The pressure sensor 116 and controller housed within unit 60 can be made operable to cause repeated intermittent pressure due to head height measurements to be taken in case the patient shifts or moves during therapy.

In the case of a continuous pumping system, such as a peristaltic pumping system, there are no intermittent points of zero velocity. In such a case, the measured pressure is assumed to be different than that expected by the above equation. The offset is compensated for in software.

In either case, the pressure drop through the flow lines is determined so that the pressure needed to run the pump 100 to achieve a desired pressure at the patient 18 can be adjusted. The measured pressure is used to maximize flow rates by maximizing pump pressure and at the same time ensuring that the pressure at the patient 18 is maintained within safe operating limits. The safety of patient 18 is the prime consideration in system 10 of the present invention. Safe pumping is maintained by not exceeding fluid pressure limits for the fluid connection point 46 of the patient's peritoneal cavity. The maximum pressures allowable at the connection point 46 of patient 18 have historically been set at +3 psig and −1.5 psig. It should be appreciated, however, that different manufacturers can have different settings and that the present invention is not limited to any particular positive or negative pressure safety settings. Those numbers, however, will be used herein to describe the method and apparatus of the present invention.

The following examples illustrate the method of using sensor 116 to correct for pressure due to head height. The valves and pump create a static fluid path in between the pressure sensor 116 and patient 18. A pressure due to head height of +0.5 psig is measured in this example. That static fluid pressure corresponds to a head height of point 46 relative to unit 60 or pump 100 of:
pressure due to head height÷(ρ×g)=h=0.5 psi÷(1 gm/cm³÷×g)=13.84 inches (0.351 m) above the instrument.

Ignoring interperitoneal pressure, head height differential means that a pressure measured at the instrument or chamber 106 reads +0.5 psig higher than the pressure measured at point 46 of patient 18 who is at the above described height above the instrument. Therefore, when draining fluid from patient 18, for example, with a fluid velocity of zero, the measured pressure is +0.5 psig higher than the pressure at point 46.

If during the drain the desired pressure at patient 18 is −1.5 psig, and knowing that the patient is 13.84 inches above the pump, the fluid pressure created at the pump needs to be controlled to −1 psig when the velocity of fluid is zero. That is, because the patient's fluid connection is elevationally above the instrument, the pressure due to head height “helps” the pump drain the patient 18 and therefore to achieve a desired negative pressure at the patient 18, the pump can use a lower pressure as measured at sensor 116 to draw fluid from the patient at the desired −1.5 psig. Conversely, if the patient is elevationally below the instrument, the pump would have to work harder or pump at a lower negative pressure to achieve the desired negative pressure at the patient, e.g., −1.5 psig.

In another example, if the measured pressure at sensor 116 is 1.0 psig (indicating that point 46 of patient 18 is 27.68 inches above unit 60), and the machine is currently in a fill mode, the fact that the patient 18 is above the machine mandates that the pump work harder to pump fluid to arrive at the patient 18 at the desired maximum allowable pressure of, e.g., +3.0 psig at point 46. For example, if the maximum pressure at the patient is +3.0 psig, the instrument would have to pump at +4.0 psig to overcome the +1.0 psig pressure due to head height and fill the patient at the maximum flow rate generating pressure of +3.0 psig. Conversely, if the patient is 27.68 inches below the pump or apparatus 50, the pump only has to operate at +2.0 psig to develop a maximum flow rate generating pressure of +3.0 psig at patient 18.

Because fluid velocity when the peristaltic pump is pumping is not zero, a pressure differential will exist in the tubing leading from chamber 106 to point 46 of patient 18. The pressure drop through a known length of tubing and possibly through a known number of elbows, tees or other types of fluid flow connectors is known. The overall pressure drop due to fluid restrictions can be calculated or estimated. That pressure drop then can be factored into the overall equation for determining the proper pressure at which to pump from the peristaltic pump of system 10. The fluid pressure at the peristaltic pump is controlled by the speed at which drive shaft 84 and rollers 80 are rotated by the motor inside the motor/valve actuator unit 60.

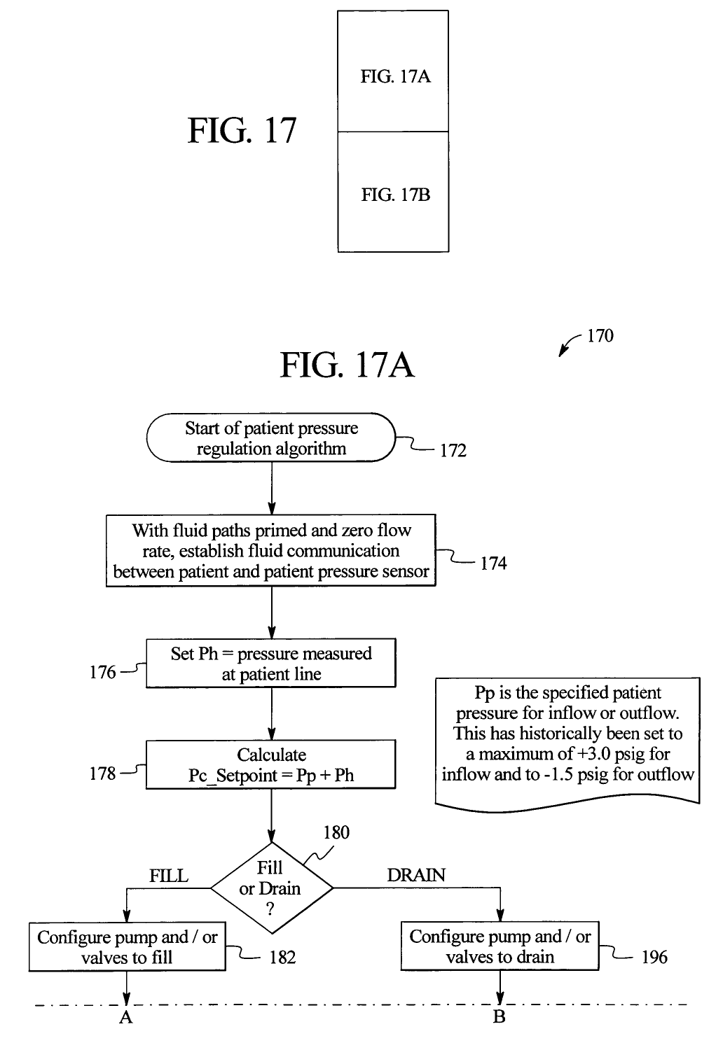

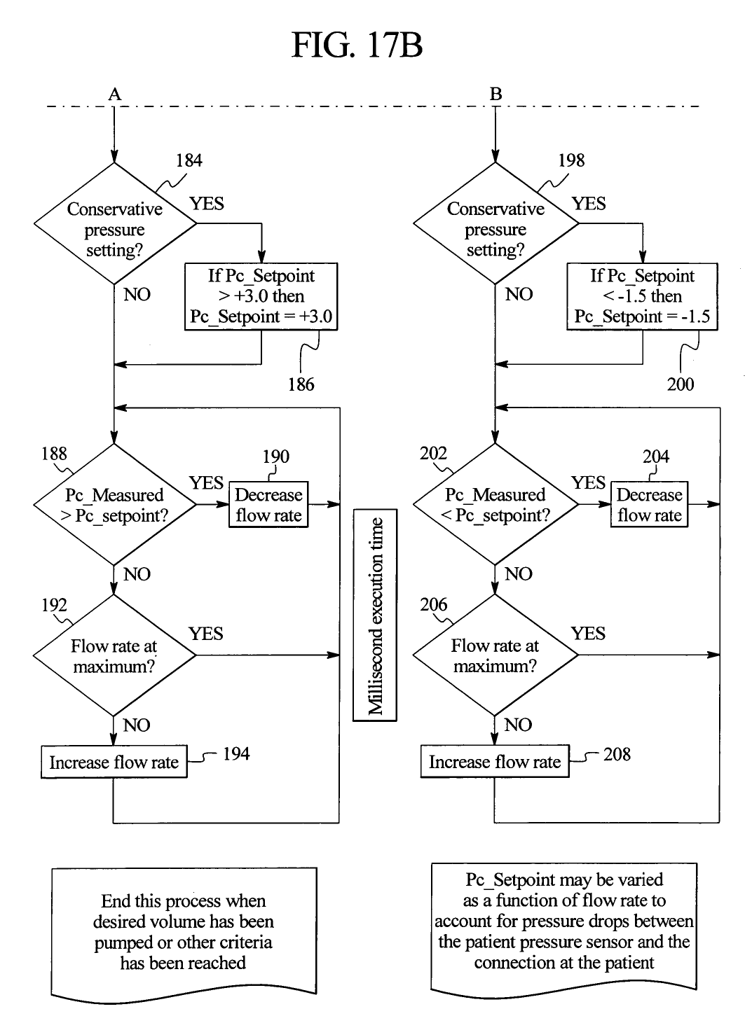

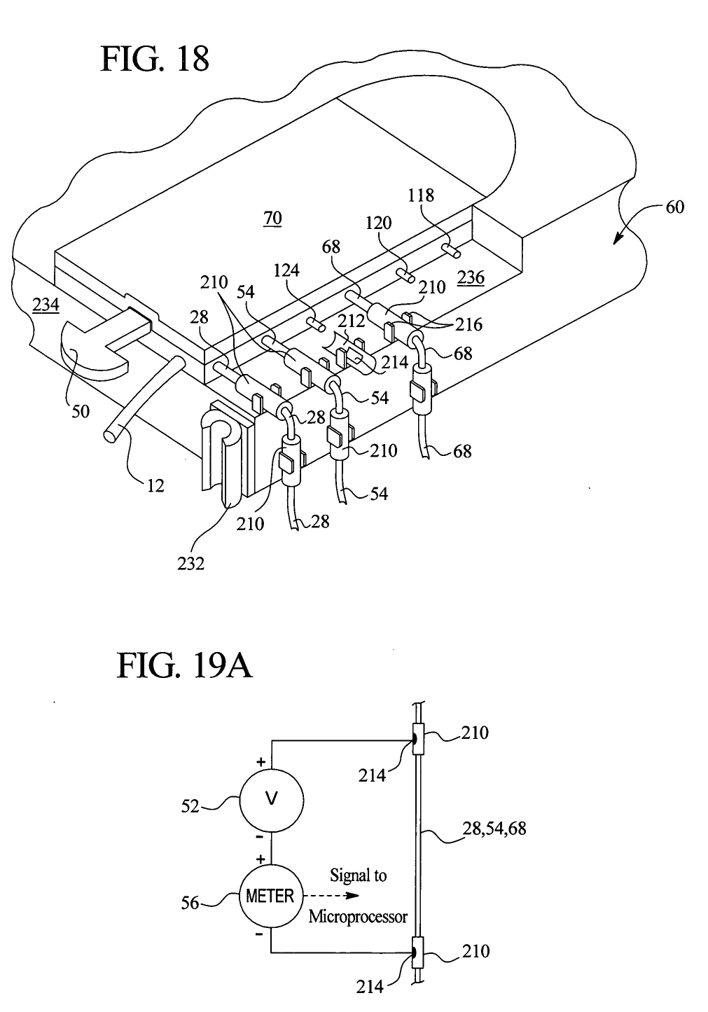

While the head height pressure sensing and calibrating method and apparatus of the present invention are shown as being operable with a peristaltic pump, it should be appreciated that a peristaltic pump is not needed to make the method and apparatus work. The method and apparatus can work with any type of fluid pump, such as a diaphragm pump. Examples of diaphragm pumps that can operate with the pressure sensor and method are disclosed in U.S. Ser. No. 10/155,754, assigned to the assignee of the present invention, entitled “Medical Fluid Pump,” the entire contents of which are incorporated herein by reference. In particular, the disclosure in connection with FIGS. 17A and 17B in that application illustrate a mechanically operated piston pump-type diaphragm pump, while the disclosure in connection with FIG. 18 illustrates a fluidly or pneumatically operated diaphragm pump.

Pneumatic, mechanical or electromechanical type diaphragm pumps are well-suited to operate with the head height apparatus and method. Indeed, because those pumps include a fixed volume chamber separated by a moveable diaphragm, the fixed volume chamber can be used in conjunction with a fluid sensor to sense the pressure against the diaphragm used in the diaphragm pumps. For example, the diaphragm can be made to contact the pressure sensor through a mechanical or pneumatic source. The pressure head height measurement can then be taken to determine the height differential between the patient and the pump. While the pressure sensor is positioned at the pump in one diaphragm pump embodiment, it is alternatively positioned upstream or downstream from the diaphragm pump.

Referring now to FIG. 17, one embodiment of the head height adjustment method of the present invention is illustrated by method 170. Method 170 begins with the patient pressure regulation algorithm loaded in software, as indicted by oval 172. After fluid paths have been primed, fluid communication is established between the patient 18 and pressure sensor 116 at a zero flowrate, as indicated by block 174. The value Ph is measured and set in software as the static pressure measured by sensor 116 through patient line 12 (when fluid velocity equals zero), as indicated by block 176.

The operating or setpoint pressure Pc is calculated to be the specified patient pressure Pp plus measured pressure Ph, as indicated by block 178. The specified patient pressure Pp is different for inflow or outflow and is generally that which has been accepted historically over multiple successful treatments. Historically, Pp has been set to a maximum of +3.0 psig for inflow and −1.5 psig for outflow to remove fluid from patient 18. Those specified pressures could be different.

Method 170 operates differently depending on whether the current cycle of system 10 is an inflow or outflow (fill or drain) cycle, as indicated by diamond 180. If in a fill cycle, the pump 100 and valves are configured to fill the patient, as indicated by block 182. Next, it is determined whether a conservative pressure setting is to be used, as indicated by diamond 184. If a conservative pressure setting is to be used and the calculated setpoint Pc is greater than 3.0 psig, then the calculated setpoint Pc is set to Pp or +3.0 psig, as indicated by block 186.

Next, or if a conservative pressure setting is not to be employed, method 170 determines whether Pc measured is greater than Pc setpoint, as indicated by diamond 188. Pc measured is the pressure at the cycler as measured during pumping. If Pc measured is greater than Pc setpoint, the flowrate is decreased by a controlled increment as indicated by block 190. The loop created by the comparison indicated by diamond 188 and the incremental flow decrease as indicated in block 190 is repeated until Pc measured is not greater than Pc setpoint, as indicated by diamonds 188 and 192.

As indicated by diamond 192, the flowrate is compared with the maximum flowrate. If the flowrate does not equal the maximum flowrate, the flowrate is increased incrementally as indicated by block 194 and the entire loop beginning at diamond 188 is repeated. Also, if the flowrate is currently at the maximum flowrate, as indicated by diamond 192, the entire loop beginning at diamond 188 is repeated. The process ends when the desired fluid volume has been pumped to patient 18 or another condition occurs, such as an alarm condition.

In a drain cycle, as indicated by diamond 180, the pump and valves are configured to drain as indicated by block 196. Next, method 170 looks to determine if a conservative pressure setting is programmed, as indicated by diamond 198. If so, and if Pc setpoint is less than −1.5 psig, then Pc setpoint is set to −1.5 psig, as indicated by block 200. Next, or alternatively in the case where a conservative pressure setting is not set, it is determined whether Pc measured is less than Pc setpoint as indicated by diamond 202.

If Pc measured is less than Pc setpoint, the flowrate is incrementally decreased, as indicated by block 204. The loop created via diamond 202 and block 204 is repeated until Pc measured is greater than Pc setpoint. At that time, it is determined whether the flowrate is at a maximum rate, as indicated by diamond 206. If not, the flowrate is increased incrementally, as indicated by block 208, and the entire loop beginning at diamond 202 is repeated. Also, if the flowrate is currently at the maximum rate as determined in connection with diamond 206, the loop beginning at diamond 202 is also repeated.

Method 170 ensures maximum flow within safe conditions, which is desirable. It should be appreciated that Pc setpoint may be varied as a function of flowrate to account for pressure drops between the patient pressure sensor 116 and the connection 46 at patient 18, which have been discussed above. One or both Pc setpoint and maximum flowrate (in fill and/or drain) can be set as a range to provide some hysteresis in system 10 to prevent continuous hunting, i.e., the flowrate from being changed continuously. The safety limits in one preferred embodiment are not compromised and are set firmly. Further, for peristaltic or continuous pumping, the flowrate loops can be interrupted periodically to reset the sequence at Ph. That feature looks to see if the patient has changed head height position and can be triggered: (i) automatically, e.g., after a specified period of time or after a specified number of strokes or (ii) upon a sudden change in pressure.

### Inline Mixing Method and Apparatus

FIGS. 1, 3, 4 and 5 illustrate another aspect of the present invention, which includes a method for mixing two supply fluids. As discussed above, in certain medical applications, such as with PD, certain solution formulations cannot be stored in mixed form for extended periods. For example, constituents of solutions made from very high and low (or differing) constituent pH fluids need to be kept separate. Cassette 50 can accept the same or different fluids through supply tubes 28, 54 and 20. FIG. 1 shows that tube 28 enables fluid to flow from supply receptacle 22 to cassette 50. Fluid line 54, on the other hand, enables flow to cassette 50 of a second different fluid, e.g., a fluid having a different pH level than the fluid within receptacle 22. Still a third different fluid, with a different pH or other property than the first and second fluids, can be introduced via bag 14 and line 20.

The fluid mixing method and apparatus in one embodiment uses a different flow chamber 112 than the flow chamber 106 described above. Chamber 112 in one embodiment is used with an inlet pressure sensor 116. Additionally, chamber 112 in one embodiment is sized and arranged to be a mixing chamber for the different fluids introduced through tubes 28, 54 and 20. In that regard, chamber 112 may include baffles or other types of mixing obstructions that cause the different fluids entering the chamber to be mixed before proceeding through pump 100 and tube 12 to patient 18. For ease of illustration, those baffles and obstructions are not shown, however, such baffling or obstructing is known to those of skill in the art and those of skill in the art should appreciate how to supply such baffles within chamber 112. Furthermore, the rigid flow path 140 leading from valve 40, which is the initial mix point for all three supplies, can include baffles or turbulators.

For purposes of the present invention, chamber 112 is assumed to define a volume when full of fluid equal to V. The flow path 110 (FIG. 6), which extends from each of valves 26, 40 and 42 to path 114 to chamber 112 and also to gatekeeper valve 44 defines a volume that is some proportion or multiple of volume V. In one example, the volume defined by each flow 110 is equal to ½ V. Such proportioning can be controlled by manipulating one or more length and/or diameter of individual flow paths 110/26, 110/40 and 110/42 to make the cumulative path 110 have a desired volume.

In one preferred flow arrangement, an outlet from mixing chamber 112 is provided that flows to valve 44 instead of the illustrated arrangement in which chamber 112 is a static volume extending from line 114. In the alternative arrangement, fluid from valves 26, 40 and 42 would be forced to travel through chamber 112 to reach valve 44, ensuring proper mixing. For example, the positions of valve 40 and chamber 112 could be switched, so that the each of the flow paths leading from valves 26, 40 and 42 runs to chamber 112. A single outlet from chamber 112 would then run to valve 44.

It should be appreciated that the volume within flow path 110 can be any desired proportion of the volume V. Also, the volume V is equal in one embodiment to a volume of fluid that is pumped through the peristaltic pump in one or more controlled increments. For example, the volume V of chamber 112 in one embodiment can be the volume of fluid that is pumped by the peristaltic pump due to one full revolution of drive shaft 84 or the rollers 80.

Alternatively, the volume of fluid pumped via one full turn of shaft 84 or rollers 80 could be equal to ½ V or the volume of the flow path 110. Further alternatively, the volume pumped by the peristaltic pump can be equal to the volume V in chamber 112 or the volume in flow path 110 based upon any controllable portion of a rotation of shaft 84 or rollers 80 or based upon any multiple controllable rotations of shaft 84 or rollers 80. For example, ½ V can equal the volume pumped by ¼ turn of shaft 84 or rollers 80 or two turns of the shaft 84 or rollers 80. Importantly, the method and apparatus operates under the assumption that after a controllable amount of pumping has taken place, e.g., a known amount of rotation of the drive shaft 84 or rollers 80 of the peristaltic pump, a known amount of fluid has entered flow path 110.

For purposes of illustration only, the following example mixes only two fluids through ports 128 and 126 and valves 26 and 42. Further, a controllable pumping unit, such as one revolution, five revolutions or ten revolutions of shaft 84 or rollers 80 is assumed to pump a volume V of fluid. Still further, the volume of flow path 110 is assumed to be ½ V.

To mix the two different fluids through paths 110/26 and 110/42, valve actuators 104/26 and 104/42 are alternated so that fluid alternatingly flows from, e.g., inlet tube 28 and then from inlet tube 54. Valve actuators 104 can, like the diaphragm pump actuators described above, be mechanically, electromechanically or pneumatically operated. If, for example, five revolutions of shaft 84 cause a volume of V to be pumped, the valve actuators 104 can be alternated every five revolutions so that upon each five revolutions, a different fluid completely fills flow path 110 and displaces another ½ V of fluid from chamber 112. That new fluid entering chamber 112 is pre-mixed via the baffles or simply the preceding common flow path into the preexisting fluid that is not dispelled from chamber 112. In that way, a controlled amount of fluid is entering the chamber at any given time.

For two fluids and equal valve increments, the amount of each fluid flowed within chamber 112 is equalized after every two pump strokes. That is, if chamber 112 is initially completely filled with fluid A, for example to prime the system, and then a volume V of fluid B is pumped in through its associated valve chamber 26 or 42, one-half of the volume V fills fluid path 110 and the other half fills of volume V fills one half of the chamber 112, leaving the remaining chamber half filled with fluid A. Next, a volume V of fluid A is pumped through its respective valve 26 or 42, so that fluid path 110 fills with fluid A, while another half of volume V of fluid A mixes with the previous mixture of A and B, creating a mixture having more A than B. When B is then pulled again into the system, the mixture will balance out and so on. Importantly, the fluids are being metered at the desired proportion on an overall basis and mixed within chamber 112. Advantageously, the fluids are being mixed inline, without adding an additional pump with its associated expense and maintenance.

To prime the system, a valve 26 or 42 controlling a priming fluid is opened and the peristaltic pump 100 is moved so that at least one-half volume V of solution is pulled into the pump. That ensures that the common flow path 110 is filled with priming solution. Thereafter, the mixing cycle described above can begin.

The two different solutions are completely mixed in chamber 112, whereafter the mixed solution is pumped to the patient. By repeating the alternating operation n times, a total volume of 2 nV of mixed solution is delivered directly to the patient. In the above example, since the pump strokes are performed by a common pump system using equally timed valves and assuming an equal pump speed, an overall one-to-one mix ratio consisting of a fluid A volume of nV and a fluid B volume of nV is very accurately achieved.

It should be appreciated that a number of alternative embodiments are possible with the flow mixing apparatus and method of the present invention. First, the volume of the flow path can be modified and/or the switching of the valves can be modified to achieve any desired proportioning ratio. Moreover, the accuracy of the volume ½ V of the flow path 110 relative to the volume of the chamber 112 is not critical because the result of any inaccuracy would be to have slightly different mixes on a per stroke basis. Overall, the proportion of the fluid delivered to the patient would be accurate. As stated above, the differences would balance every two pump strokes or controlled pump revolutions so that the mix of the fluid entering the patient's peritoneum is accurate.

The method of the present invention is not limited to two fluids but is alternatively extendable to any suitable number of different fluids. The mixing can also be done with proportions other than one-to-one, such as two-to-one, three-to-one, three-to-two, etc. In such a case, the controlled pumping stoke mixing could cause the ratio within chamber 112 to vary more, however, the overall ratio mixed and delivered to the patient's peritoneum would be correct after each repeated cycle. Such degree of mixing is generally acceptable for dialysate concentration mixing, especially where two solutions only differ by concentration.

Further, by changing the valve state the proportions can be changed. For example, the first valve could be opened for only a single pumping revolution, after which the second valve could be opened for two, three or more revolutions or some fraction of a revolution to achieve the desired ratio. The volume of chamber 112 may in such cases have to be modified to ensure that some mixing takes place within the chamber upon each sequencing of the valves. Chamber 112 can be located upstream or downstream from pump 100, regardless of whether pump 100 is reversible or not.

It should also be appreciated that, as with the head height compensation aspect of the present invention, the inline mixing feature is not limited to peristaltic pumping but is also readily applicable to pumping with a diaphragm pump. Indeed, in such a case, the additional chamber 112 may not be needed, where a fixed volume chamber associated with the diaphragm pump is used instead. In that case, flow path 110 feeds immediately into a diaphragm pump, wherein the volume of the flow path is some proportion of the volume of the pump.

### Inline Solution pH Measurement

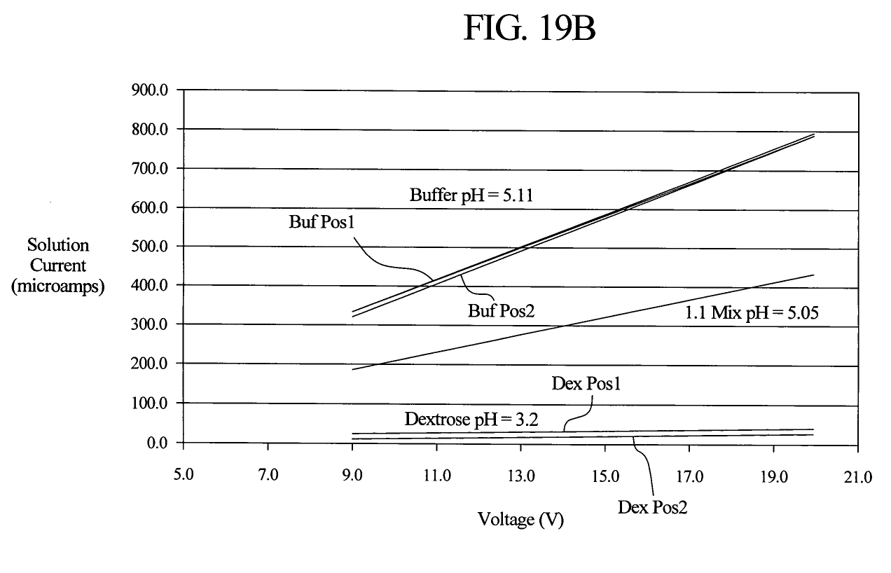

Referring now to FIGS. 18 and 19A, one apparatus and method for determining inline the pH values of dialysate and components thereof is illustrated. The apparatus and method are particularly useful for admixing situations in which dialysate components having different pH values are mixed at the point or time of use. It should be appreciated, however, that the apparatus and method are not limited to admixing situations and may be used in any case to confirm the proper pH value of dialysate being delivered to patient 18. The apparatus and method are non-invasive. The pH values of different dialysate components are measured and compared to expected values. For example, if dextrose is being introduced, e.g., via supply line 28 into cassette 50, the pH can be checked to confirm the proper pH of about 3.2.

For each fluid sensed, the apparatus includes a pair of conductive electrical connectors 210 that are spliced between two sections of tube, such as two sections of tube 28, tube 54 or tube 68 as illustrated. Connectors can be connected to the tubes in multiple ways, such as being welded, solvent bonded, radio frequency (“RF”) sealed, compression fitted or threaded to the tubes.

The materials suitable for connector 210, as well as other information relating to the signal generation and feedback system are described in more detail in U.S. Ser. No. 10/760,849, entitled, “Conductive Polymer Materials and Application Thereof Including Monitoring and Providing Effective Therapy,” filed Jan. 19, 2004, which is a continuation-in-part application of U.S. Ser. No. 10/121,006, filed Apr. 10, 2002, the entire contents of each of which are hereby incorporated by reference. One preferred formulation for the conductive polymer fitting 210 is discussed below.

The conductive tubing fitting 210 in the illustrated embodiment clips or snap-fits into a holder 212, which is sized and shaped to firmly hold connector 210. Holder 212 includes one or more conductive electrodes 214 that contacts and electrically couples to conductive fitting 210. The connector 210 is held in place by spring clips 216, which in one embodiment are non-conductive. Connector pairs 210 are illustrated as being operable with supply tubes 28 and 54 and heater tube 68. It should be appreciated that connector pairs 210 can be provided with any one or more or all of the tubes provided with cassette 50.

As seen in FIG. 19A, electrodes 214 are in turn wired to or placed in electrical communication with a voltage source 52 and a conductivity meter 56 or other type of current or conductivity measuring and signal generating device. Source 52 induces a voltage on the circuit. Tubes 28, 54 and 68 are generally non-conductive. Accordingly, current must flow through the fluid traversing through tubes 28, 54 and 68 to complete the circuit. The meter 56, as illustrated, senses the level of current or conductivity of the fluid and sends a signal indicative of same to the controller or microprocessor within unit 60 of system 10. The controller or microprocessor receives the signal from electrode 214 and connector 210 and inputs the signal into a closed loop feedback system.

As seen in FIG. 19B, it has been found that different solutions exhibit different and distinct current outputs based on varying voltage inputs. The Pos 1 and Pos 2 curves for dextrose and buffer represent outputs received from contacting fittings 210 at with electrodes 214 at two different places to test to see whether connectors 210 will having varying outputs due to non-uniformity. The results show that fittings 210 can be contacted at different places and still yield good results. It should be appreciated that the scales used for voltage and current are illustrative and not limiting. The applied voltage in actual operation can be less than or more than the range shown in FIG. 19B. The current output will vary accordingly.

Knowing the voltage induced, and sensing the current through the circuit of FIG. 19A, and knowing the expected pH and the expected conductivity or current reading, system 10 can determine if the solution is at the proper pH value. If, for example, dextrose is supplied via supply tube 28 and a buffer is supplied through supply tube 54, connectors 210 can respectively monitor that the dextrose has the proper pH value of about 3.2 and that the buffer has the proper pH of about 5.1. The controller or processor knows the desired admix ratio, e.g., 1:1. In such a case, the controller or processor knows that the resulting pH should be 5.05. The admixed solution is measured at tube 68 in FIG. 18, which in one embodiment is the tube running to heater 38. It is desirable to measure the pH before the dialysate is heated.

In one embodiment, the material for conductive fitting 210 is acrylonitrile butadiene styrene (“ABS”) with stainless steel fiber-filled composites. The loading of stainless steel can be from about 1% by volume to about 35% by volume. The use of stainless steel maintains the medical grade of the equipment, while providing for a conductive filler.

As illustrated, the different pH values of the different solutions enable a different amount of current to flow through connectors 210, electrodes 214 and the remainder of the circuit of FIG. 19A. The output signal of meter 56 to the controller or microprocessor includes current or conductivity. The conductivity, for example, is then correlated to a particular pH value, wherein the microprocessor can interpret a signal, determine a pH value based on a conductivity reading of the signal and compare the pH correlation with an expected correlation. The conductive fitting 210 and electronic circuitry thereby provide a safety feature to insure that the pH values of the inputted components of the dialysate and/or of the resulting dialysate itself are proper.

### Air Detecting and Removal Method and Apparatus

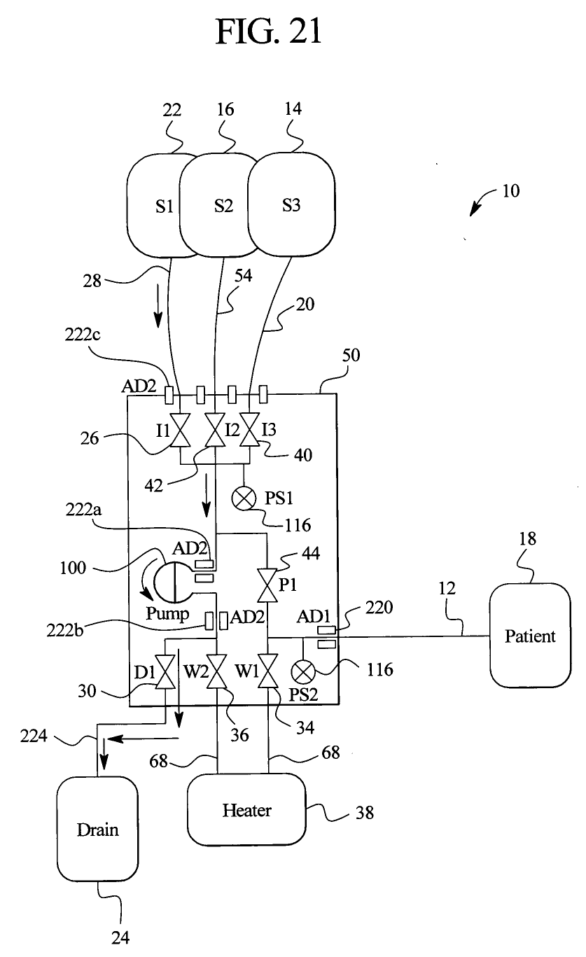

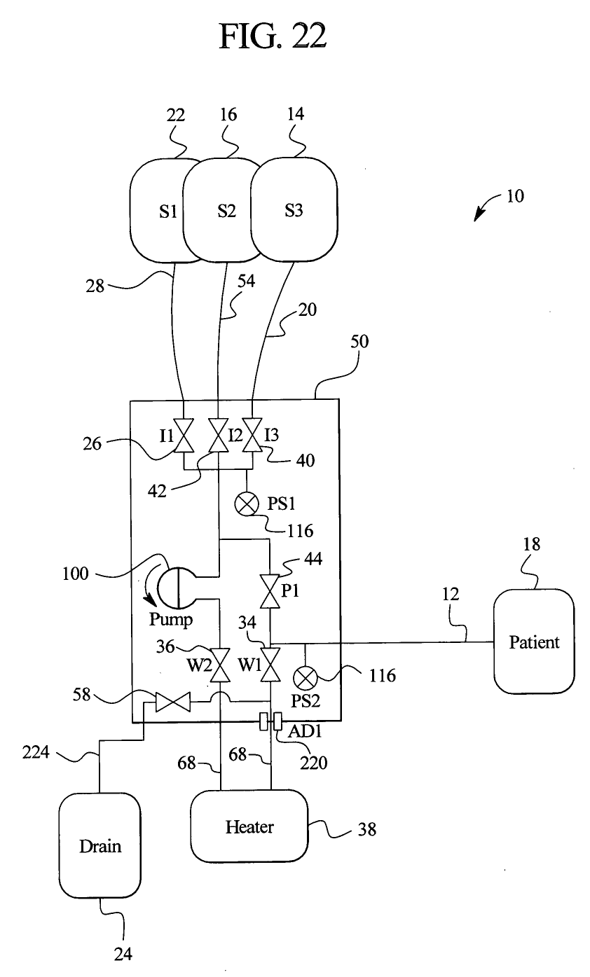

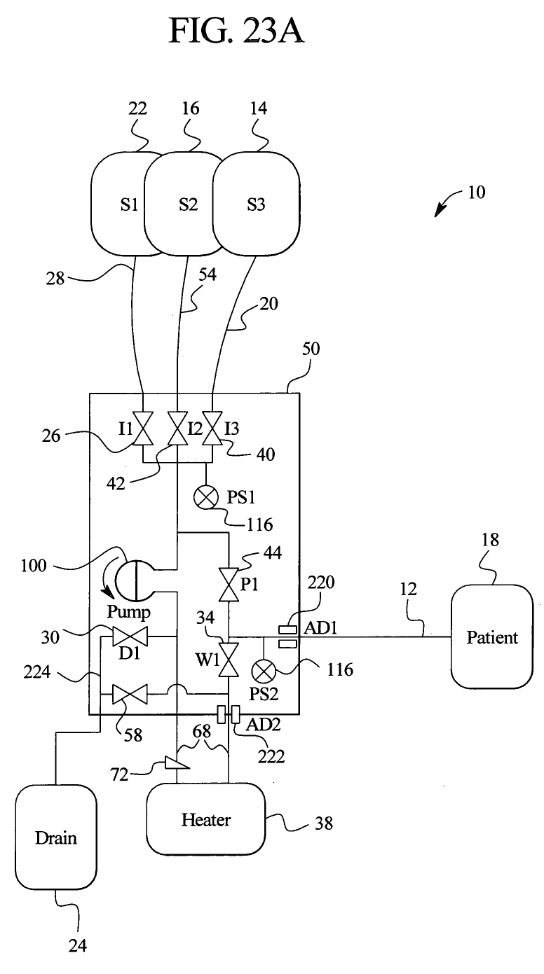

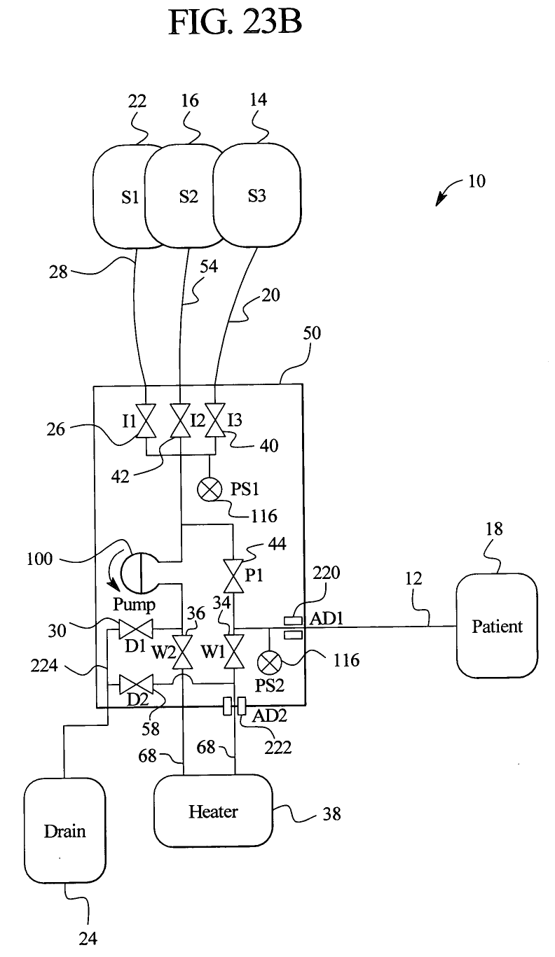

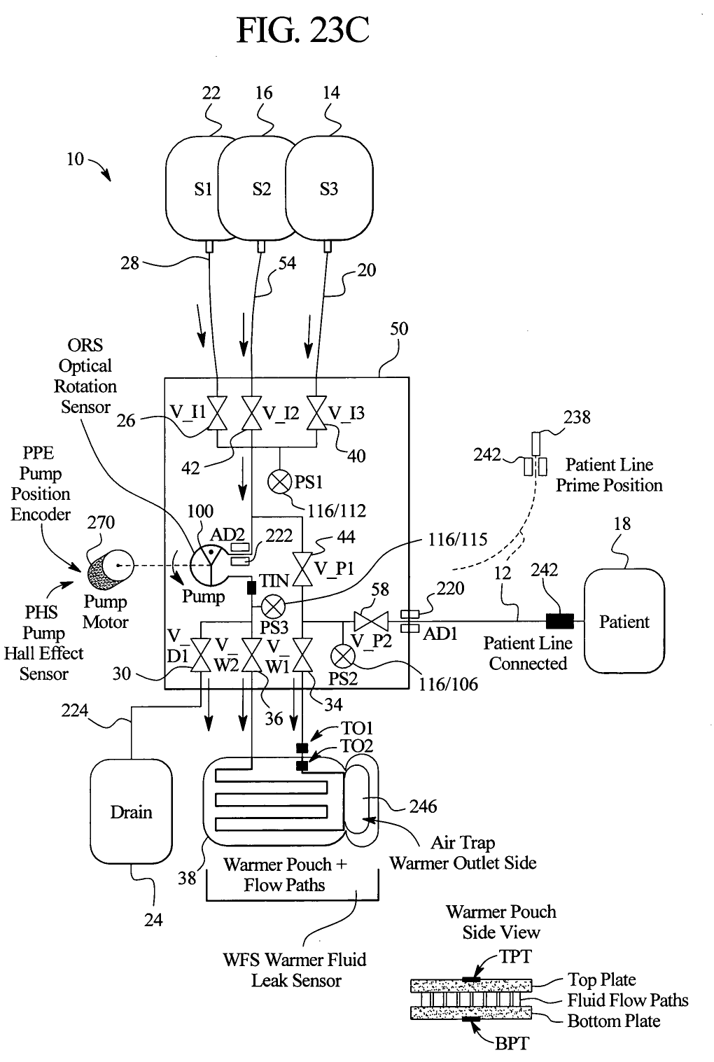

Referring now to FIGS. 20, 21, 22, 23A and 23B, various alternative embodiments for an air detecting and removal apparatus and method are illustrated. Each of FIGS. 20 to 23B includes various components that have been described previously in detail. Briefly recounting those elements, FIGS. 20 to 23A and 23B each include three supply bags 22, 16 and 14 that are coupled to valves 26, 42, and 40, respectively, via supply fluid lines 28, 54 and 20. FIGS. 20 to 23A and 23B show schematically the flow paths illustrated in FIGS. 4 to 6. In particular, it is seen that a pressure sensor 116 described above is positioned fluidly to sense the inlet pressure of the supply fluids. The fluid supply flows past supply pressure sensor 116 and is pumped through pump 100. Certain of the figures also illustrate that pump 100 can pump either to drain 24 through valve 30 or instead through heater 38 via valves 34 and 36. FIGS. 22, 23A and 23B show a different flow path arrangement than FIGS. 20 and 21, as described in more detail below.

Valves 26, 42, 40, 34, 36, 30 and 44 operate with pump 100 to enable fluid to be sent to or pulled from patient 18. That is, if valves 44 and 30 are closed and valves 36, 34 and one of valves 26, 42 or 40 are open, pump 100 in a fill mode pumps fluid through heater 38 to patient 18. Alternatively, if valves 26, 42, 40, 36 and 34 are closed, isolating heater 38 (in FIGS. 20 and 21), and valves 44 and 30 are open, pump 100 pumps fluid from patient 18 to drain 24.

The introduction of air or excessive air into the peritoneal cavity of patient 18 is considered a safety hazard resulting, among other things, in pain in the patient's extremities. Because of that safety hazard, prevention of air infusion is a requirement of the system 10 of the present invention. There are different sources of areas where air can enter system 10. Air is automatically present in cassette 50, patient line 12 and the various solution lines when those items are sterilized and prior to prime. That air needs to be primed from the system as discussed in more detail below. There also exists sterilized air in the solution bags because it is typically very difficult to fill the entire volume of the solution bag with fluid only. Non-sterile air can also enter the dialysate solution circuit via an unclamped or unconnected line or via an improper connection or leaky connection. Further, non-sterile air can enter the dialysate circuit of system 10 due to a leak between the membrane 102 and cassette 50 or via a tear or leak in membrane 102.

Regarding the sterile air in the cassette, that air needs to be removed prior to therapy. Further, all air including non-sterile air needs to be detected and purged from the system to protect the patient and to prevent contamination. As described in more detail below, system 10 primes the solution circuit with dialysate to remove air from the lines addressing one of the sources noted above.

Regarding air that enters the system due to a leaky connection, an unclamped or unconnected line or through a leaking cassette or tube, that air needs to be detected prior to the initial fill of the patient. The leaking cassette and tubing must be discarded and replaced prior to the initial fill of the patient to prevent potentially contaminated solution from being pumped to the patient. To that end, system 10 performs one or more integrity tests on membrane 102 prior to therapy, which alerts system 10 if membrane 102 of cassette has a leak. System 10 also uses sensor 222 during flush or prime and air sensor 220 during an initial drain to alarm and stop therapy if air continues to be sensed during those procedures.

Regarding sterile air that enters the system via the solution bags, that air is typically sensed towards the end of the current fill cycle when the supply bag has been largely emptied. At that time sterile air in the supply bags is prone to being pumped into cassette 50. System 10 provides the following apparatus and method for detecting such air and for removing it from the dialysate circuit before resuming therapy. As seen in FIGS. 20, 21, 23A and 23B, it is known in a peristaltic APD system to provide an air sensor 220 at the exit of cassette 50 to sense fluid flowing through patient line 12 to patient 18. That location is desirable in one aspect because sensor 220 is sensing air at the last possible point in the cassette prior to fluid being delivered to patient 18. In that manner, sensor 220 senses any air entering the dialysate circuit prior to that point.

The location of air sensor 220 has certain disadvantages, however, such as: (i) there is no detection of air in the circuit until after it has been pumped through the heater and towards patient 18; (ii) the air that is detected is at the front end of the air bubble or stream of bubbles, so that there is no way to determine the amount of air that has entered the dialysate circuit behind the bubble or portion of air that has been sensed; (iii) when air is detected, while it is possible to pull the air back from the patient line 12 and move it to drain 24 by running the drain sequence, the flow in the system is stopped while the air is purged, causing fluid in the heater 38 to become overheated, so that it must be cooled before being delivered to patient 18; and (iv) if air is present in the form of a stream of bubbles, which it often is, the solution will require many short drain and fill cycles to purge the air.

FIGS. 20 and 21 illustrate that a second air sensor 222 (collectively referring to sensors 222a to 222c) is placed in the dialysate circuit to operate in combination with sensor 220 described above. FIGS. 20 and 21 show three possible positions for the second sensor 222. Each of the positions provides for a detection of air in the dialysate circuit between solution bags 14, 16 and 22 and the valves 30 and 36 that determine the destination of supply fluid traveling from those bags through pump 100.

The first position shown by sensor 222a is one preferred position. Detecting the air at the position shown by sensor 222a enables all air originating from the solution bags, solution lines or fluid paths up to the point of sensor 222a to be easily diverted to drain 24 with minimal loss of fluid and time. In particular, as fluid is being pumped to patient 18, if sensor 222a detects air, valve 34 and/or 36 is closed and valve 30 is opened to enable or divert the flow of fluid to drain 24. Such valve switching prevents detected air from reaching the heater 38 or patient 18. The air laden dialysate is pumped to drain 24 until sensor 222a detects that the air bubbles or stream has passed, at which time drain valve 30 is closed and heater valves 36 and 34 are opened to again enable fresh solution to be pumped to patient 18.

The position of second air sensor 222 can alternatively be placed as indicated by sensor 222b. The location of 222b provides less reaction time to stop flow to patient 18 and to switch valves to divert flow to patient 24, however, the location of air sensor 222b would detect any air entering via peristaltic pump 100, e.g., through the connection of tube 76 to ports 96 and 98 as seen FIGS. 4 and 7. Further, the location of sensor 222b would detect any air entering via an imperfection or leak in peristaltic pumping tube 76.

The third location shown by sensor 222c actually includes multiple sensors, which each check one of the supply lines 28, 54, and 20. The advantage here is more reaction time and the fact that the sensors could be located outside of cassette 50, perhaps in a similar manner shown above with conductive fittings 210 in FIG. 18, so that cassette 50 does not have to be modified substantially and/or enlarged. The disadvantage is that multiple sensors are needed and that air introduced via inlet valves 26, 42 or 40 or pump 100 would not be detected.

For each of the positions for second air sensor 222, FIG. 20 illustrates that in normal operation fluid flows through pump 100, through valve 36, through heater 38, through valve 34, passing sensor 220, through patient line 12 and to patient 18. FIG. 21 illustrates that for each different position of second sensor 222, if air is detected, one or both valves 36 and 34 is closed, and drain valve 30 is opened, directing flow instead to drain 24.

FIG. 22 illustrates a further alternative embodiment for detecting air prior to a point at which the fluid and air can no longer be diverted to drain 24. In FIG. 22, the first sensor 220 is moved from the position shown in FIGS. 20 and 21 to the position shown in FIG. 22, which senses the fluid flowing through heater line 68 exiting heater 38 and reentering cassette 50. Further, the drain line 224 is switched to communicate fluidly with line 68 downstream of the placement of air sensor 220. In this configuration, drain valve 30 is removed and a second drain valve 58 is added as a by-pass valve from the outlet 68 of heater 38 to drain line 224 and drain 24. To purge air from the system 10 in FIG. 22 during the fill cycle, valve 34 is closed and valve 58 is opened to divert air laden dialysate to drain 24. The advantage in FIG. 22 is that only a single air sensor 220 is used. The disadvantages are that the flow path of the cassette 50 has to be changed and there is no protection for the patient 18 for any leaks caused at valve 34 or via the exit pressure sensor 116. Further, the air is detected after the fluid is heated, which will result in a slight waste of system energy. It should be appreciated, however, that the position of air sensor 220 also has the advantage of purging or diverting dialysate which has been overheated.

Referring now to FIG. 23A, one placement of second air sensor 222 is illustrated. Here the sensor is placed in the same position along line 68 as shown by first sensor 220 in FIG. 22. This is desirable because the sensor 222 interfaces with a tube 68 rather than at an intermediate point within cassette 50. In this configuration, heater valve 36 is removed and a second drain valve 58 is added as a by-pass valve from the outlet 68 of heater 38 to drain line 224 and drain 24. To pump fluid to patient 18, valves 30, 58 and 44 are closed, while valve 34 and one of valves 26, 42 or 40 are opened. To pump fluid from patient 18 to drain 24, valves 34 and 58 are closed, while valves 44 and 30 are open. To purge air from the system IO in FIG. 23A during the fill cycle, valve 34 is closed and valve 58 is opened to divert air laden dialysate to drain 24. In one embodiment, an external clamp 72 is provided on input heater line 68. Clamp 72 occludes heater line 68 during flush and prime. System 10 can be configured to ensure that clamp 72 is closed prior to flush.

Referring now to FIG. 23B, another valve arrangement is illustrated. In FIG. 23B, sensor 222 is again placed in the same position along line 68 as shown previously in FIG. 23A. This is desirable because the sensor 222 interfaces with a tube 68 rather than at an intermediate point within cassette 50. In this configuration, heater valve 36 remains and the second drain valve 58 is added as a by-pass valve from the outlet 68 of heater 38 to drain line 224 and drain 24. To pump fluid to patient 18, valves 30, 58 and 44 are closed, while valves 34, 36 and one of valves 26, 42 or 40 are opened. To pump fluid from patient 18 to drain 24, valves 34, 36 and 58 are closed, while valves 44 and 30 are open. To purge air from the system 10 in FIG. 23A during the fill cycle, valve 34 is closed and valve 58 is opened to divert air laden dialysate to drain 24. External clamp 72 is not needed in FIG. 23B.

The system of FIG. 23B allows air to be directly diverted to drain 24 when detected during fill and flow to be maintained through the heater 38 in the event of an air detection, preventing an over temperature fluid condition due to stopped flow in the heater 38. FIG. 23B requires an extra sensor 222 and valve 58, however, extra sensor 222 is positioned at the tubing and does not require cassette modification.

Referring now to FIG. 23C, an eight valve configuration of cassette 50 of system 10 is illustrated. Here, a second air sensor 222 (as in FIGS. 20 and 21) is placed directly in front of pump 100. FIG. 23C shows pump motor 270 operating with pump 100. An optical rotation sensor (“ORS”) operates with pump 100 to detect, for example, the position of pump rollers, the number of peristaltic pump shaft rotations, etc. Motor 270 likewise operates with a Hall Effect sensor (“PHS”) and positional encoder (“PPE”). The PHS can detect a position of the shaft of motor 270. PPE gives positional and potentially velocity and acceleration information about the shaft of motor 270.

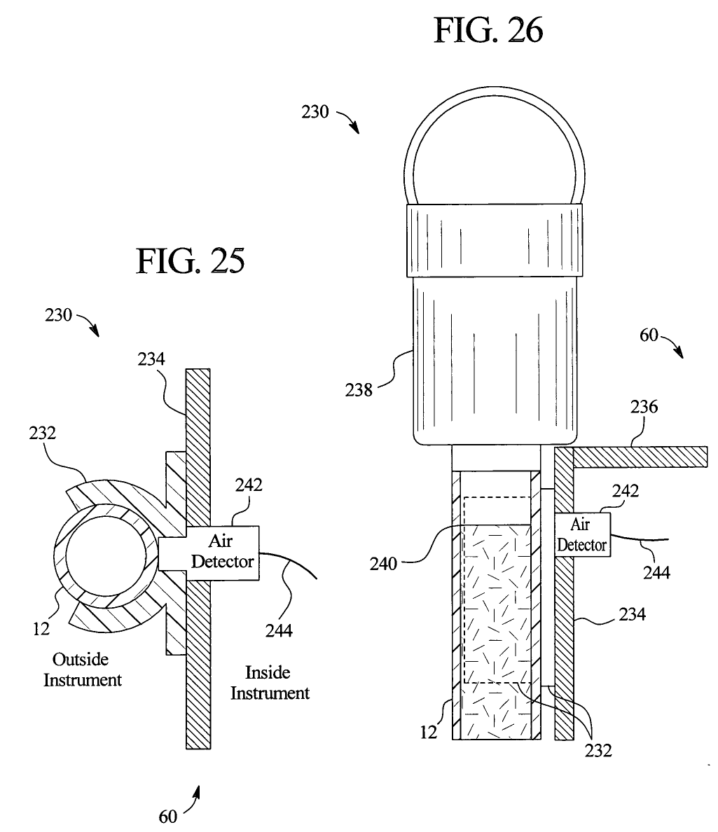

A third air detector 242 is placed at the end of patient line 12, just before tip protector 238 (see FIGS. 25 and 26). The patient line prime position shown in FIG. 23C is discussed in detail below in connection with FIGS. 25 and 26. An air trap 246 is provided with warmer pouch 38. In an embodiment, warmer pouch 38 is disposed vertically with respect to unit 60.

System 10 of FIG. 23C is generally the same as for FIGS. 20 and 21. The drain and heater valves and paths are the same as is the placement of sensors 220 and 222. System 10 of FIG. 23C adds eighth valve 58, which further isolates fluid exiting heater 38 from patient 18. The valving arrangement of FIG. 23C creates three zones of isolation. A first zone exists between inlet valves 26, 40 and 42 and the inlet of pump 100 (as shown above, rollers 280 of the pumps occlude pump tubing 76 in a valve like manner). A second zone exists between the outlet of pump 100, drain 24 and the inlet of heater 38. A third zone exists between the outlet of heater 38, bypass valve 44 and patient 18.

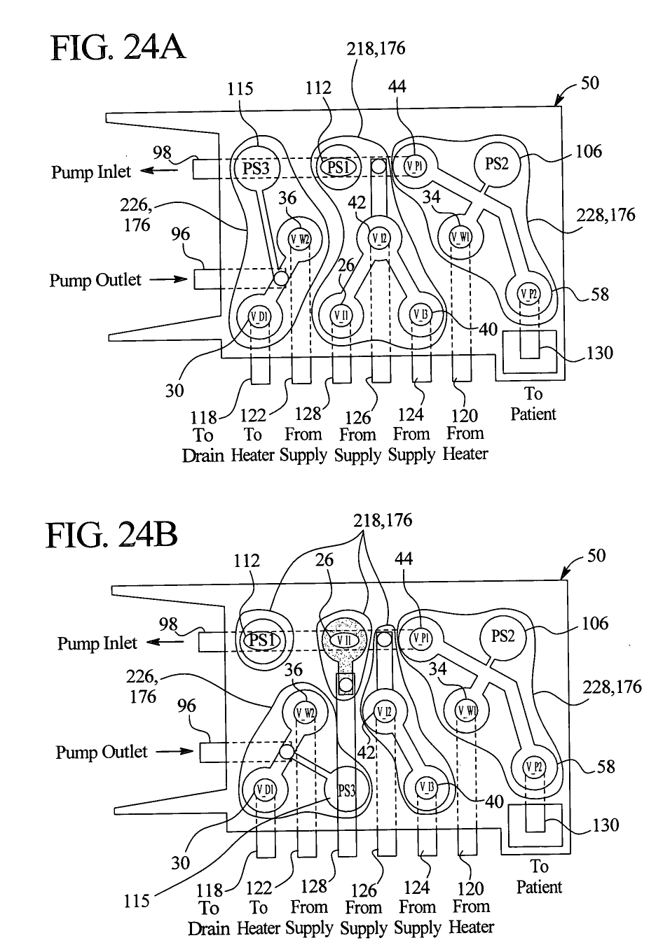

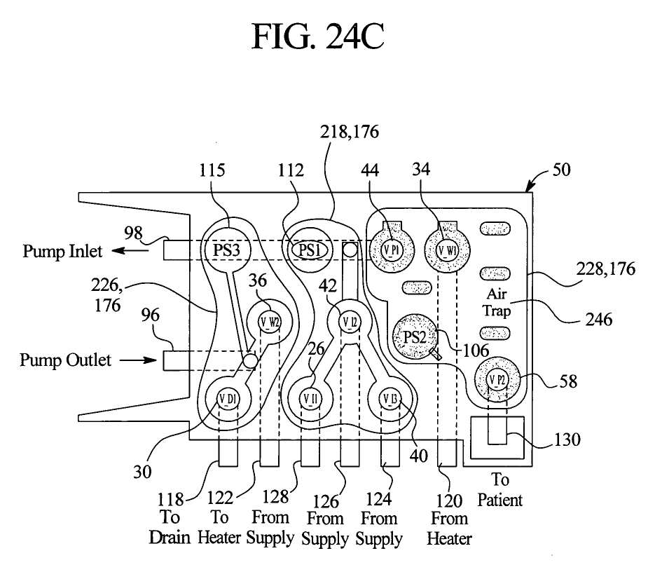

Referring now to FIGS. 24A to 24C, three cassettes 50 each show a different zoned flowpath arrangement for the valves and pressure sensors of system 10 of FIG. 23C. The three cassettes 50 are advantageous in one respect because each of the valve ports 118, 120, 122, 124, 126, 128 and 130 extend from a single side of cassette 50 (compare, e.g., with FIG. 4, in which patient port 130 extends from a different side of cassette 50 than valve ports 118, 120, 122, 124, 126 and 128). Reducing the number of sides from which the ports extend reduces the number of molding “pulls,” or directions in which dialysate flows in and out of cassette 50. This is advantageous because the molding tool for cassette 50 is simpler with fewer side actions or “pulls.”

The three cassettes 50 are advantageous in another respect because of the above-referenced zones. In each FIG. 24A to 24C, a first zone 218 encompasses inlet valves 26, 40 and 42, flow chamber 112 (which operates with first pressure sensor 116 as seen in FIG. 23C) and the inlet 98 to pump 100. The dotted lines in FIGS. 24A to 24C indicate flow paths that reside elevationally beneath the solid lined flow paths. As seen in FIG. 5, these flow paths are formed integrally with cassette 50 via the “pulls” on the molding tool. The flow paths shown in solid in FIGS. 24A to 24C reside elevationally above the pulled flow paths and are defined collectively by the rigid piece of cassette 50 and flexible membrane 102.

Bypass valve 44 also enables liquid to communicate between zones 218 and 228. In each FIG. 24A to 24C, a second zone 226 encompasses drain valve 30, to heater valve 36 and chamber 115 (which operates with third pressure sensor 116). In each FIG. 24A to 24C, a third zone 228 encompasses from heater valve 34, bypass valve 44, to patient valve 58 and fixed volume chamber 106 (which operates with second pressure sensor 116).

Cassettes 50 complete the enclosed zones by isolating the dialysate between the cassette body and flexible membrane 102 via the sealing ribs 176 (described above), which form the topside perimeter of zones 218, 226 and 228, as illustrated. Each zone isolates a common node shown in FIG. 23C and is isolated from the other flow paths, preventing cross talk. Each zone 218, 226 and 228 includes its own pressure sensor, and a set of isolating valves, which adds a diagnostic tool for determining where a leak might be if one is sensed. “To patient” valve 58 enables zone 228 (and the other zones) to be isolated from patient 18 so that the leak tests can be performed properly and safely. The “to patient” valve 58 also allows the warmer bag air trap 246 to be emptied, if needed, during treatment. To empty the warmer bag air trap 246, valves 30, 44 and 34 are opened and pump 100 is rotated until the air trap is deflated and air is transferred to drain 24.

As seen in FIG. 24C, cassette 50 in one embodiment includes an integral air trap 246. Air trap 246 is located in zone 228 in one embodiment because the zone is downstream of heater 38 and located just upstream of patient 18. Air trap 246 is moved from the warmer bag 38 of FIG. 23C to the cassette 50 of FIG. 24C. Here, cassette 50 is positioned vertically within unit 60 for operation. In the illustrated embodiment, air trap 246 is formed by removing specific flow paths within zone 228, which creates a larger volume in that zone. Standoffs or internal ribbing can be added within zone 228 to support the membrane 102 if needed, e.g., if zone 228 is under negative or positive pressure.

In the vertical orientation, the patient valve 58, which enables dialysate to flow to patient 18 via port 130, is protected from air bubbles that may enter zone 228 from warmer 38 and from heater valve 34. Air bubbles that enter zone 228 through valve 34 remain at the top of air trap 246 within zone 228, while dialysate flows from the bottom of zone 228 to patient 18. The volume of air trap 246 is sufficient to hold a typical amount of air that might be generated during a patient fill phase without increasing the size of cassette 50. System 10 automatically removes air from trap 246 during the next drain phase because bypass valve 44 is located at the top of the air trap 246. During the drain phase, as solution flows into the air trap 246 from the patient 18, the air is pushed out of trap 246, through bypass valve 44 and to drain 24.

If it happens that air fills air trap 246 completely during the patient fill phase, air can escape through valve 58, but is detected by air detector 242 in patient line 12 as illustrated by FIG. 23C. Such detection signals system 10 to: (i) cause pump 100 to stop, (ii) switch valves to a patient drain configuration (valves 58, 44 and 30 open) and (iii) pump enough dialysate from the patient line to drain line to flush the air out through valve 44 and zone 228. Filling can then be quickly resumed without over heating fluid in heater 38.

### Active Patient Line Priming Apparatus and Method

Referring now to FIGS. 1, 18, 25 and 26, one apparatus and method of the present invention for actively priming patient line 12 is illustrated by the system 230. As discussed above, prior to connecting system 10 to patient 18, patient line 12 is primed with dialysate to remove any air from that line. An incomplete prime leaves air in the line, which may be infused into patient 18, potentially causing a safety hazard. Patient line 12 in operation is typically 10 to 20 feet (3 to 6 meters) in length, to provide mobility and comfort to patient 18 during treatment.

FIGS. 25 and 26 illustrate that patient line 12 is loaded into or snap-fitted into a patient line holder 232. One possible placement of patient line holder 232 is to be fastened to or formed by wall 234 of instrument 60, shown in FIG. 18. Thus when patient 18 loads cassette 50 into actuator unit 60 and closes door 70, the patient also snap-fits patient line 12 into patient line holder 232 as shown in FIG. 18.

FIG. 25 illustrates a top view of patient line holder 232 and patient fluid line 12 at a section beneath the patient fluid line connector 238 shown in FIG. 26. Patient fluid line connector 238 helps patient 18 to properly position patient fluid line 12 in holder 232. As illustrated in FIG. 26, patient 18 places line 12 into holder 232 so that connector 238 just rests on top of top wall 236 shown also in FIG. 18. That position aligns a desired fluid priming level, shown by line 240, with an air detector 242 embedded in wall 234 and extending through a portion of holder 232 as seen in FIG. 25.

Air detector 242 includes a signal line 244 that extends to the controller or processor housed inside actuator unit 60. Air detector 244 senses the following conditions or states: (i) an unloading condition where holder 232 is empty, that is, patient tubing line 12 has not yet been placed in holder 232; (ii) a loaded and dry condition where patient tubing line 12 has been placed in holder 232 but line 12 is still dry (unprimed); and (iii) a loaded and wet condition where patient line 12 has been placed in holder 232 and pump 100 has pumped solution to the fluid prime level indicated by line 240.

Air detector 242 can be any type of air detector known to those of skill in the art, such as an ultrasonic, capacitive, inductive or optical type of sensor. Because sensitivity is not a critical factor, and cost is a factor, one preferred type of air detector is an optical detector.

During the set up of system 10 for therapy, if the unloaded condition is sensed, system 10 will prompt patient 18, e.g., via display 66, to load the patient line 12 into holder 232. System 10 then waits until the patient does so, at which point the loaded but dry condition is sensed.

Once air detector 242 senses the loaded but dry condition, the processor or controller in system 10 causes pump 100 and valves of instrument 60 to pump an initial amount of solution, less than the minimum line length, to patient line 12 at a slow rate to place a certain amount of fluid into line 12. Thereafter, pump 100 pumps at an even slower rate until the air sensor 242 senses a change from the loaded but dry condition to the loaded and wet condition.

If the loaded and wet condition is not sensed by sensor 242 after pump 100 pumps fluid into patient tubing line 12 over a predetermined time, or, after a predetermined volume of fluid is pumped, system 10 stops pumping and prompts the patient or operator, e.g. via display 66, whether an extension line is being used. In certain instances, the patient adds an additional tubing length to the end of the standard tubing length provided with cassette 50. In that case, the above sequence of dosing of an initial volume into line 12 and pumping of incremental fluid volume does not work because the initial volume does not come close enough to the end of line 12. In such case, system 10 assumes that an extension has been added and seeks conformation of same from the user. If system 10 confirms that an extension line has been used, e.g., after receiving a “yes” input via display 66 or controls 62 and 64 on instrument 60, system 10 will resume pumping up to a second predetermined maximum value, and thereafter pump incrementally until the loaded and wet condition is reached.

In an alternative embodiment, controls 62 and 64 and display 66 are configured to enable the patient or operator to enter upfront the fact that an extension line is being used, e.g., through an “extension line” set up menu. If a patient enters in the set up mode that an extension line has been added, system 10 causes pump 100 to automatically pump to the second maximum value without the above-described intermediate pumping and prompting. At the second maximum, incremental pumping occurs until the loaded and wet condition is sensed.

While FIG. 18 shows holder 232 being oriented vertically, there is no requirement that such be the case, and holder 232 is alternatively oriented at any desired angle. For example, it may be desirable to orient holder 232 horizontally so that the patient fluid line 12 does not extend directly vertically below instrument 60 due to holder 232, which could cause problems in attempting to lay instrument 60 flat on a table.

It should also be appreciated that detector 242 can employ many different types of technologies. As stated above, detector 242 can be optical or ultrasonic. Even within the optical branch sensors, however, there are variations, each of which could be used for sensor 242. For example, an optical sensor could utilize the transmissive properties of the dialysate and the tubing by orienting a transmitter/receiver pair on opposite sides of holder 232. Alternatively, an emitter and receiver can be housed in a single unit and used in combination with a reflective member placed on the opposite side of holder 232 from the emitter/receiver device. Further, the position of holder 232 may be incorporated into a larger type of fluid line organizer, which is constructed and arranged to guide the operator or patient 18 through the proper sequence of connecting the various fluid tubes at the beginning of therapy.

### Volumetric Accuracy Improvement Method

System 10 employs at least one algorithm that enables the volumetric accuracy of pump 100 to be more accurately determined. While the following volumetric accuracy improvement method is applicable to peristaltic pumping, it should be appreciated that the method is also applicable to diaphragm pumps and other types of positive displacement pumps. Ideal positive displacement pumps, using only rigid, well-defined movements, theoretically deliver a fixed volume of incompressible fluid over every stroke, which could be easily and accurately calculated and summed.

In the case of a diaphragm pump or a peristaltic pump tube, non-rigid components are used, causing the volume of fluid displaced over every pump cycle to change or potentially change. The change is a function of many factors, including the deformation of the non-rigid components and the varying forces acting on those components. Thus, the volumetric accuracy is at least a function of the material properties and the forces acting on the material. Further, when the motor mechanism used to drive the positive displacement pump is not operated exactly as expected, the imprecise movement or positioning may affect the overall volume of fluid displaced. The method described herein teaches how to overcome the over-described inaccuracies, as well as others. The method in essence includes identifying factors that can cause error in the volumetric pumping system. Each factor is isolated and a correlation between the factor and an amount of introduced error is determined either theoretically or empirically. A constant K is calculated or determined based on the correlation between the factor and the error. Finally, an overall volumetric equation, for example, an equation corresponding to volume pumped by a diaphragm pump or a peristaltic pump, is modified to include a product of the determined constant K multiplied by a value for the factor.

In some instances, the value of the factor is inputted. For example, the factor could be a tubing material that is inputted into the system 10, wherein the factor takes into account various properties of the material. On the other hand, the value of the factor can be measured. For example, the factor could be an inlet pressure to a peristaltic pumping tube or a pressure differential across the diaphragm of a diaphragm pump, which needs to be actually measured within system 10. The method of the present invention enables a more accurate algorithm to be used than to simply either: (i) sum the number of pump strokes for a diaphragm pump; or (ii) count the number of revolutions for a peristaltic pump.

The method of the present invention is advantageous over previous methods for calculating the volume of a diaphragm pump, which included an assumption that the volume was fixed for each displacement or stroke of the pump. For example, it would be assumed that the diaphragm pumping chamber would be filled completely with fluid. It is known, however, that the fluid contains a certain amount of air. Therefore, over time, less fluid would actually be pumped than would assumed to have been pumped, due to the fact that part of the overall volume pumped was consumed by an amount of air. The method of the present invention takes this factor into account in determining the overall volume of fluid pumped by, for example, measuring the amount of air that is typically present within any given pump stroke. That amount of air can then be scaled for different pumping pressures to determine an overall volume of fluid that is assumed to be displaced, but in actuality is not displaced, for any given pump cycle. That volume is then subtracted from the overall volume equation to produce a more accurate algorithm.

In another example, it is assumed with diaphragm pumps that the mechanism for driving the diaphragm, such as a pump piston, always moves from the bottommost possible portion of the stroke to the topmost possible portion of the stroke and vice versa. In actuality, the pump piston likely does not move all the way to the top or all the way to the bottom, at least not in every stroke. Therefore, to increase accuracy, the actual position of the piston head or diaphragm can be measured, for example, by a rotary or linear encoder attached to the pump piston head. In either case, the result is more accurate than simply presuming that the pump moves each time to the commanded uppermost and lowermost positions of the pump stroke.

Besides air and pump stroke errors, diaphragm pumps are also affected by other characteristics of the pumping instrument, such as the material of the diaphragm and the differential pressure across the diaphragm. After creating a constant for each one of those factors, an overall algorithm is determined that takes into account as many factors as can be analyzed and correlated. An example equation is as follows:
volume pumped in a diaphragm pump=K1×PSME²+K2×PSME+K3×dP²+K4×dP+K5,
where each constant K is found during the experimentation or calibration process. PSME (piston stepper motor encoder position) is the piston position for a diaphragm pump employing a stepper motor-drive piston. dP is the differential pressure across the diaphragm which, in one embodiment, is the chamber fluid pressure minus vacuum pressure behind the diaphragm. It should be appreciated that while the above equation is more accurate than simply multiplying the number of pump strokes by an assumed volume pumped per each stroke, the above equation can be modified from the form in which it appears to be made more accurate, or less accurate, depending upon the effort involved in determining the constants K1 to K5, as well as the hardships in measuring certain of the factors, such as pressure or temperature.

Known methods to calculate the volume pumped by a peristaltic pump include simply counting the number of revolutions of the pump head or shaft 84 or by multiplying the rotational velocity commanded by the running time of the pump 100. Both of those methods will, over time, introduce error. Those equations assume that every rotation of the pump head displaces a fixed volume of fluid from the inlet of the pumping tube 76 to the outlet of same. Those methods also assume that the inlet and outlet fluid pressures, tubing temperature, rotational speed of the pump head, tubing wear, tubing properties, roller wear, along with tubing dimensions, do not effect the volume of fluid transported by the rollers acting on the tube segment. Because those factors can have an affect on overall volume pumped, a more accurate algorithm would take into effect at least some of those factors.

The algorithm for the peristaltic pump can be specified to be the volume of fluid displaced per revolution, or vol/rev. As the pump head rotates, at least one roller and associated occlusion moves along the tube and fills the tube from the fluid source until the next roller occludes the tube in a second location on the tube. The first and second rollers trap fluid between the two occlusions and move the volume to the outlet of the tube. Any variable affecting the inner diameter dimensions of the tubing up to the point when the volume of fluid is trapped would, or could, affect the vol/rev of the pump.

For example, a negative inlet pressure may not allow the tube to return to the same shape as when the inlet pressure is zero. The negative inlet pressure may therefore lower the vol/rev pumped. Alternatively, positive inlet pressures may increase the inside diameter of the tube, versus the diameter that would be present if the inlet pressure is zero, and thereby increase the vol/rev. Further, tubing temperature could also lower the vol/rev by prohibiting the tube from returning to its original shape, at least over a short amount of time, for example, when the pump is being run at a high rate of speed. The tubing will typically become distorted over long periods of pumping, as described above, further affecting the vol/rev. Tubing -wear can be predicted by the properties of the tubing or measured empirically. Roller wear can be measured empirically and factored into the overall peristaltic pumping volumetric equation, which can vary over time, or vary the wear constant K based on the total number of pump revolutions over the life of the machine.

The method of improving volumetric accuracy for a peristaltic pump, such as pump 100, accounts for or estimates the above-mentioned error variables and modifies the vol/rev calculation based on a function that models the effects of the variables by determining a constant for each variable. Then values are inputted or measured for variables. For example, fluid pressures at the inlet and outlet of the pumping tube 76 can be measured with a pair of pressure sensors, such as sensor 116. Furthermore, the fluid temperature can be measured, to thereby calculate an average temperature of the tubing. The rotational speed of the pump motor can be assumed to be correct if the pump motor is accurate enough or alternatively measured by a tachometer.

An example of a volume per revolution algorithm for a peristaltic pump is:
vol/rev=(K1×Pin²+K2×Pin)×(K3×temp²+K4×temp)×(K5×speed²+K6×speed)+K7,
where K1 to K7 are constants to be determined empirically or via data. Pin equals the fluid pressure at the inlet of the peristaltic pumping tube 76. Temp. equals the average temperature of the tube. Speed equals the rotational driveshaft speed of the pump, which is either entered or measured. The total volume displaced by the pump would then be the number of revolutions multiplied by the vol/rev calculation, where the number of revolutions is counted, for example, by an encoder or other type of positional sensing device.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

1. A peristaltic pump comprising:
a member that is moved across a section of tubing to compress the tubing and thereby move fluid through the tubing; and
wherein the tubing has a Shore A Hardness in a range of about 50 to about 85 and a tear resistance of about 110 to about 480 in-lb/in.

2. The peristaltic pump of claim 1, wherein the tubing is selected from the group consisting of: high quality silicone, silicone blend, ethylene propylene diene monomer (“EPDM”), polyurethane (“PU”), polyvinylchloride (“PVC”), ultra-high molecular weight PVC (“UHMWPVC”), styrene block copolymer, metallocene-catalyzed ultra-low density polyethylene (“m-ULDPE”), polytetrafluoroethylene (“PTFE”) and any combination thereof.

3. The peristaltic pump of claim 1, wherein the tubing exhibits at least one fluid volume accuracy selected from the group consisting of: (i) at least about 90 percent for a fluid having a pH of about 9.0 and after being pumped for at least about 12 hours; (ii) at least about 90 percent for a fluid having a pH of about 2.0 and after being pumped for at least about 12 hours; and (iii) at least about 90 percent for a fluid being pumped from a head height of at least about ±0.5 meters.

4. The peristaltic pump of claim 1, wherein the tubing has at least one characteristic selected from the group consisting of: (i) a substantially uniform diameter, (ii) a substantially consistent wall thickness, (iii) is accurate over a temperature range of about 40° C., (iv) a compression set in a range of about 20% to about 85% at 73° C. and 22 hours, and (v) is sealable to a disposable cassette.

5. A peristaltic pump comprising:
a member that is moved across a section of tubing, enabling the tubing to be compressed and expanded, thereby moving fluid through the tubing; and
wherein the tubing exhibits a fluid volume accuracy of at least ninety percent for a fluid having a pH of about 2.0 to about 9.0, and wherein the fluid has been pumped through the tubing from a head height of at least about ±0.5 meters for at least 12 hours.

6. The peristaltic pump of claim 5, wherein the tubing is selected from the group consisting of: high quality silicone, silicone blend, ethylene propylene diene monomer (“EPDM”), polyurethane (“PU”), polyvinylchloride (“PVC”), ultra-high molecular weight PVC (“UHMWPVC”), styrene block copolymer, metallocene based on ultra-low density polyethylene (“m-ULDPE”), polytetrafluoroethylene (“PTFE”) and any combination thereof.

7. The peristaltic pump of claim 5, wherein the tubing has at least one characteristic selected from the group consisting of: (i) a Shore A Hardness in a range of about fifty to about 85, (ii) a tear resistance of about 110 to about 480 in-lb/in, (iii) is accurate over a temperature range of about 40° C. and a compression set in a range of about 20% to about 85% at 73° C. and 22 hours, and (iv) is sealable to a disposable cassette.

8. A disposable dialysis apparatus comprising:
a disposable cassette providing at least one flow path, at least one valve chamber in fluid communication with the flow path, and a plurality of ports; and
tubing in fluid communication with the ports, the tubing forming a loop operable with a peristaltic pump, and wherein the tubing has a Shore A Hardness in a range of about fifty to about 85 and a tear resistance of about 110 to about 480 in-lb/in.

9. The disposable dialysis apparatus of claim 8, wherein at least one of the tubing and the disposable cassette is sterilized via a process selected from the group consisting of: an ethylene oxide rinse and radiation.

10. The disposable dialysis apparatus of claim 8, wherein the tubing is mated to the ports via at least one process selected from the group consisting of: molding, extrusion molding, solvent bonding, friction fitting, radio frequency sealing, heat sealing, laser welding and any combination thereof.

11. The disposable dialysis apparatus of claim 8, wherein the tubing is selected from the group consisting of: high quality silicone, silicone blend, ethylene propylene diene monomer (“EPDM”), polyurethane (“PU”), polyvinylchloride (“PVC”), ultra-high molecular weight PVC (“UHMWPVC”), styrene block copolymer, metallocene based on ultra-low density polyethylene (“m-ULDPE”), polytetrafluoroethylene (“PTFE”) and any combination thereof.

12. The disposable dialysis apparatus of claim 8, wherein the tubing exhibits at least one fluid volume accuracy selected from the group consisting of: (i) at least about ninety percent for a fluid having a pH of about 9.0 and after being pumped for at least about 12 hours; (ii) at least about 90 percent for a fluid having a pH of about 2.0 and after being pumped for at least about four hours; and (iii) at least about 90 percent for a fluid being pumped from a head height of at least about ±0.5 meters.

13. The disposable dialysis apparatus of claim 8, wherein the tubing has at least one characteristic selected from the group consisting of: (i) a substantially uniform diameter, (ii) a substantially consistent wall thickness, (iii) a compression set in a range of about 20% to about 85% at 73° C. and 22 hours, and (iv) is accurate over a temperature range of about 40° C.

14. A disposable dialysis apparatus comprising:
a disposable cassette providing: (i) at least one flow path, (ii) at least one valve chamber in communication with the flow path, (iii) a pair of ports, and (iv) an air trap; and
tubing in fluid communication with the ports, the tubing forming a loop that operates with a peristaltic pump, wherein the pump is operable to pump fluid exiting the cassette, and wherein air from the fluid is collected in the air trap.

15. The disposable dialysis apparatus of claim 14, wherein the tubing (i) is selected from the group consisting of: high quality silicone, silicone blend, ethylene propylene diene monomer (“EPDM”), polyurethane (“PU”), polyvinylchloride (“PVC”), ultra-high molecular weight PVC (“UHMWPVC”), styrene block copolymer, metallocene-catalyzed ultra-low density polyethylene (“m-ULDPE”), polytetrafluoroethylene (“PTFE”) and any combination thereof, (ii) has at least one characteristic selected from the group consisting of: a Shore A Hardness in a range of about fifty to about 85, a tear resistance of about 110 to about 480 in-lb/in, a substantially uniform diameter, a substantially consistent wall thickness, a particulate matter (“PM”) level that is less than the PM level exhibited by silicone tubing, a compression set in a range of about 20% to about 85% at 73° C. and 22 hours and is accurate over a temperature range of about 40° C.; and (iii) exhibits a fluid volume accuracy over at least about twelve hours of at least about ninety percent for a fluid having a pH of about 2.0 to about 9.0, and wherein a fluid pumped through the tubing has been pumped from a head height of at least about ±0.5 meters.

16. The disposable dialysis apparatus of claim 14, wherein the tubing is mated to the ports via a process selected from the group consisting of: molding, extrusion molding, solvent bonding, friction fitting, radio frequency sealing, heat sealing, laser welding and any combination thereof.

17. The disposable dialysis apparatus of claim 14, which includes at least one characteristic selected from the group consisting of: (i) the air trap being located elevationally above a valve port leading to the patient; (ii) each valve port being located on a same side of the cassette; and (iii) each valve chamber being part of a zone capable of being fluidly separated from each other zone.

18. A medical fluid apparatus operable with a fluid pump, the apparatus comprising:
a disposable cassette, the cassette including a body and a flexible membrane, the body and membrane enclosing a chamber in the cassette, the chamber having a fluid inlet and a fluid outlet;
a pressure sensor coupled operably with a portion of the membrane, so as to sense pressure fluctuations of a fluid flowing through the chamber; and
electronics configured to: (i) receive a signal from the pressure sensor, the signal indicative of a head height of a patient, and (ii) use the signal to determine a pressure at which to operate the pump.

19. The medical fluid apparatus of claim 18, wherein the desired pressure is a function of at least one of: flow rate maximization and pressure limit adherence.

20. The medical fluid apparatus of claim 18, wherein the electronics have at least one characteristic selected from the group consisting of: (i) being configured to determine the operating pressure based on the pressure signal and a factor corresponding to a predicted pressure drop due to at least one flow restriction between the pump and the patient; (ii) being housed in a unit coupled with the disposable cassette; and (iii) being housed in a unit that additionally includes a pump driving mechanism.

21. The medical fluid apparatus of claim 20, wherein the driving mechanism is mechanically activated or pneumatically activated.

22. The medical fluid apparatus of claim 18, wherein the pump is a peristaltic pump and the cassette includes a tube that is coupled operably with a driving mechanism of the peristaltic pump.

23. The medical fluid apparatus of claim 22, wherein the tube: (i) is made of a material selected from the group consisting of: high quality silicone, silicone blend, ethylene propylene diene monomer (“EPDM”), polyurethane (“PU”), polyvinylchloride (“PVC”), ultra-high molecular weight PVC (“UHMWPVC”), styrene block copolymer, metallocene-catalyzed ultra-low density polyethylene (“m-ULDPE”), polytetrafluoroethylene (“PTFE”) and any combination thereof; and (ii) has at least one characteristic selected from the group consisting of: a Shore A Hardness in a range of about fifty to about 85, a tear resistance of about 110 to about 480 in-lb/in, a substantially uniform diameter, a substantially consistent wall thickness, a particulate matter (“PM”) level that is less than the PM level exhibited by silicone tubing, and a compression set in a range of about 20% to about 85% at 73° C. and 22 hours.

24. The medical fluid apparatus of claim 18, wherein (i) when the pressure due to head height is positive, the electronics are configured to set a positive operating pressure at the pump higher than a desired positive pressure at the patient to fill the patient at the desired positive pressure and to set a negative operating pressure at the pump lower than a desired negative pressure at the patient to drain the patient at the desired negative pressure and (ii) when the pressure due to head height is negative, the electronics are configured to set a positive operating pressure at the pump lower than a desired positive pressure at the patient to fill the patient and to set a negative operating pressure at the pump lower than a desired negative pressure at the patient to drain the patient at the desired negative pressure.

25. The medical fluid apparatus of claim 18, wherein the chamber is a pumping chamber of the pump.

26. A medical fluid apparatus comprising:
a pump driving mechanism;
a pressure sensor; and
electronics configured to (i) receive a signal from the pressure sensor indicative of a head height of a patient and (ii) use the signal to determine an operating level at which to operate the driving mechanism.

27. The medical fluid apparatus of claim 26, wherein the operating level is a function of at least one of: flow rate maximization and pressure limit adherence.

28. The medical fluid apparatus of claim 26, wherein the electronics are configured to determine the operating level based on the pressure signal and a factor corresponding to a predicted pressure drop due to at least one flow restriction between the pump and the patient.

29. The medical fluid apparatus of claim 26, wherein the pump is a peristaltic pump and the driving mechanism includes a head that rotates against a fluid carrying tube, and wherein the operating level is a level at which the head rotates against the tube.

30. The medical fluid apparatus of claim 26, wherein (i) when the pressure due to head height is positive, the electronics are configured to set a positive operating level at the mechanism higher than the desired positive level to fill the patient at the desired positive level and to set a negative operating level at the mechanism higher than a desired negative level at the patient to drain the patient at the desired negative level; and (ii) when the pressure due to head height is negative, the electronics are configured to set a positive operating level at the mechanism lower than a desired positive level to fill the patient at the desired positive level and to set a negative operating level at the mechanism higher than a desired negative level at the patient to drain the patient at the desired negative level.

31. A medical fluid apparatus operable with a fluid pump that pumps a fluid volume V per pumping increment, the apparatus comprising:
a mixing chamber;
first and second fluid supplies holding different first and second fluids;
a fluid path having a first end fluidly connected to an inlet of the chamber, the fluid path having a volume P, which is a predetermined portion of the volume V; and
first and second valves placed at a second end of the fluid path and controlling flow of the first and second fluids, the valves alternated so that (i) a volume P of the first fluid is pumped to the flow path and a volume V-P of the first fluid is pumped to the mixing chamber in a first pumping increment and (ii) a volume P of the second fluid is pumped to the flow path and a volume V-P of the second fluid is pumped to the mixing chamber in a second pumping increment.

32. The medical fluid apparatus of claim 31, wherein the mixing chamber is also a pumping chamber of the fluid pump.

33. The medical fluid apparatus of claim 31, wherein the pump is of a type selected from the group consisting of: a diaphragm pump and a peristaltic pump.

34. The medical fluid apparatus of claim 31, wherein the pump is a peristaltic pump having at least one of the following characteristics: (i) being located upstream of the chamber and fluid path; (ii) being located downstream of the chamber and fluid path; (iii) being operable so that the pump increments are portions of a revolution of a drive shaft; (iv) being operable so that the pump increments are a full revolution of the drive shaft; and (v) being operable so that the pump increments are multiple revolutions of the drive shaft.

35. The medical fluid apparatus of claim 31, wherein the volume P is substantially one-half the volume V.

36. The medical fluid apparatus of claim 31, wherein a volume defined by the chamber is substantially equal to the volume V.

37. The medical fluid apparatus of claim 31, wherein the valves are controlled to produce a mixture of the first and second fluids in other than a one-to-one ratio.

38. The medical fluid apparatus of claim 31, wherein the first increment is a first percentage of a complete pump cycle and the second increment is a second percentage of the pump cycle, the first and second percentages chosen to create a desired overall ratio of first and second fluids.

39. A medical fluid apparatus comprising:
a mixing chamber;
first and second fluid supplies holding different first and second fluids, the supplies in fluid communication with the mixing chamber;
a fluid pump; and
first and second valves controlling flow of the first and second liquids, the valves and the pump configured to alternatingly partially fill the chamber with the first fluid and then partially fill the chamber with the second fluid and simultaneously remove some but not all of the first fluid from the chamber.

40. The medical fluid apparatus of claim 39, which includes a pressure sensor operably coupled to a flexible membrane portion of the mixing chamber, the sensor measuring a pressure due to a relative head height position between the pump and a patient fluid connection.

41. The medical fluid apparatus of claim 39, wherein the first and second supplies are tied together to a common inlet fluid path running to the mixing chamber.

42. A medical fluid apparatus comprising:
a rigid body defining multiple flow paths, multiple valve chambers and multiple fluid ports;
a tube connected in a loop with the body, the loop sized to fit around a roller pumping head assembly of a peristaltic pump, the assembly configured to be engaged and positively and abutingly driven by a member moved by a pump motor; and
a flexible membrane sealed and coupled in a tamper proof manner to the body and enclosing the fluid paths and the valve chambers, the seal and coupling made prior to a time when the body is loaded into a pump and valve actuation instrument.

43. The medical fluid apparatus of claim 42, which includes at least one characteristic selected from the group consisting of: (i) the membrane being coupled to the body by a process selected from the group consisting of: sonic welding and mechanical snap-fitting and (ii) the body being acrylic and the membrane being polyvinyl chloride.

44. The medical fluid apparatus of claim 42, wherein at least one of: (i) the flow paths is defined at least in part by sealing ribs projecting from the body, the ribs configured to provide an airtight seal when contacted by the membrane; (ii) the ribs configured to provide multiple airtight seals that cooperate with the valve chambers to form isolated fluid zones; and (iii) at least one of the zones defines an air trap.

45. The medical fluid apparatus of claim 42, wherein the assembly includes features that interface with mating features of the member moved by the pump motor.

46. A medical fluid apparatus comprising:
a disposable cassette defining multiple flow paths, multiple valve chambers and multiple fluid ports;
a supply container connected fluidly to a first one of the fluid ports;
a drain line connected fluidly to a second one of the fluid ports;
a patient fill line connected fluidly to a third one of the fluid ports;
a peristaltic pump configured to pump fluid from the supply bag to the patient fill line or the drain line based on which valve chambers are opened and closed; and
an air sensor positioned relative to the valve chambers so that fluid from the supply container can be diverted to drain instead of being pumped to the patient if air in the fluid is detected by the air sensor.

47. The medical fluid apparatus of claim 46, wherein the air sensor includes at least one characteristic selected from the group consisting of: (i) being positioned directly upstream to or downstream from the peristaltic pump; (ii) being coupled operably to the cassette; (iii) being coupled operably to a supply line connecting the supply container to the cassette; and (iv) being a first air sensor and which includes a second air sensor coupled operably to the patient fill line.

48. A medical fluid system comprising:
a disposable cassette defining multiple flow paths, multiple valve chambers and multiple fluid ports;
a tube connected to one of the fluid ports, the tube including a conductive portion, the conductive portion configured to enable a reading indicative of the pH value of a fluid traveling within the tube to be taken; and
a processor configured to input the reading and determine if the pH value for the fluid is acceptable.

49. The medical fluid system of claim 48, wherein the conductive portion includes a conductive fitting coupled to at least one section of the tube.

50. The medical fluid system of claim 48, which includes a housing that encloses the processor and to which the cassette is mounted, the housing including a coupler configured to receive and hold the conductive portion.

51. A medical fluid system comprising:
a disposable cassette defining multiple flow paths, multiple valve chambers and multiple fluid ports;
a peristaltic pump connected fluidly to the cassette;
a patient line connected to one of the fluid ports;
a patient line holder into which the patient line is placed and held;
a sensor cooperating with the holder to send a signal indicating that: (i) the patient line has not been placed in the holder, (ii) that the patient line has been placed in the holder and fluid has not yet reached a sensible level, and (iii) that the patient line has been placed in the holder and fluid has reached a sensible level; and
a processor configured to input the signal and make at least one determination based on the signal.

52. The medical fluid system of claim 51, which includes a connector placed at the end of the patient line, the connector aiding a person to position the tube properly in the holder.

53. The medical fluid system of claim 51, wherein the sensor is an optical, ultrasonic, capacitive or inductive sensor.

54. The medical fluid system of claim 51, wherein the holder is a first holder and which includes additional holders organized to aid a person to properly initiate therapy.

55. A method of performing a dialysis procedure comprising the steps of:
installing a disposable cassette so that tubing held by the cassette is operably coupled with a pump head of a pump; and
causing the tubing to be made from a non-silicon material that enables fluid to be pumped by moving the pump head against the tubing at an accuracy of at least ninety percent over the entire dialysis procedure.

58. The method of claim 55, wherein performing the dialysis procedure includes performing in-center hemodialysis, home hemodialysis, in-center peritoneal dialysis, home hemodialysis, in-center hemodiafiltration, home hemodiafiltration, continuous ambulatory peritoneal dialysis, automated peritoneal dialysis, tidal flow peritoneal dialysis, congestive heart failure therapy or any combination thereof.

59. The method of claim 55, which includes at least one of: (i) operating the pump at a head height of at least ±0.5 meters; (ii) maintaining a pH of the fluid outside of a range of greater than 2.0 and less than 9.0; and (iii) maintaining the volumetric accuracy by controlling at least one of the following characteristics of the tubing: (a) controlling a thickness of the tubing to be uniform, (b) controlling a diameter of the tubing to be consistent, (c) controlling a length of the tubing to be consistent, (d) controlling an inner surface of the tubing to be smooth, and (e) controlling at least one of a tear resistance and compression set of the tubing so that the tubing does not deform significantly via contact with the pump head over the entire dialysis procedure.

60. A method of performing a dialysis procedure comprising the steps of:
selecting a desired fluid pressure at the patient;
sensing a pressure at a fluid pump due to a patient's head height position relative to the pump;
controlling an output pressure of the pump to compensate for the pressure; and
pumping fluid at the output level to deliver the fluid to the patient at the desired fluid pressure.

61. The method of claim 60, wherein controlling an output pressure of the pump includes at least one of: (i) operating within safe positive and negative operating limits; and (ii) compensating for pressure drop due to at least one flow restriction between the pump and the patient.

62. The method of claim 60, wherein (i) controlling the output pressure when the pressure due to head height is positive includes setting a positive operating pressure at the pump higher than the desired positive pressure to fill the patient at the desired positive pressure and setting a negative operating pressure at the pump higher than a desired negative pressure to drain the patient at the desired negative pressure; and (ii) controlling the output pressure when the pressure due to head height is negative includes setting a positive operating pressure at the pump lower than a desired positive pressure to fill the patient at the desired positive pressure and setting a negative operating pressure at the pump lower than a desired negative pressure to drain the patient at the desired negative pressure.

63. A method for performing a dialysis treatment comprising the steps of:
pumping a first fluid into a mixing chamber;
pumping a second fluid, different from the first fluid, into the mixing chamber and displacing some of the first fluid to produce a first mixture;
pumping the first fluid into the mixing chamber and displacing some of the first mixture to create a second mixture, wherein the second mixture has a proportion of first and second fluids different than that of the first mixture; and
delivering an overall volume of mixed fluid to a patient, the overall volume having at least substantially a desired proportion of first and second fluids to a patient.

64. The method of claim 63, which includes at least one additional step selected from the group consisting of: (i) measuring fluid pressure in the mixing chamber and using the measured pressure to compensate for a pressure due to head height in determining a pumping pressure; and (ii) pumping the first and second liquids through a common line to the mixing chamber.

65. The method of claim 64, which includes structuring the common line to (i) define a volume in a desired proportion to a volume defined by the mixing chamber; or (ii) define a volume in a desired proportion to a volume of fluid delivered in a controllable pumping increment.

66. The method of claim 63, wherein the mixing chamber is additionally a pumping chamber, and the pumping increment is at least a portion of a stroke of a diaphragm between walls of the chamber.

67. The method of claim 63, wherein the pumping increment is at least a portion of a rotation of a drive shaft or roller of a peristaltic pump.

68. A method for improving the volumetric accuracy in a dialysate pumping system, the method comprising the steps of:
identifying a factor causing volumetric error in pumping dialysate;
isolating the factor and empirically determining a relationship between the factor and volume of dialysate pumped;
determining a constant K for the factor using the empirically determined relationship; and
modifying an overall equation for calculating a volume of dialysate pumped by a product of the constant K and a value for the factor.

69. The volumetric accuracy improvement method of claim 68, wherein the value for the factor is measured or entered.

70. The volumetric accuracy improvement method of claim 68, wherein the pumping system is (i) a diaphragm pumping system and the factor is selected from the group consisting of: a position of a pump diaphragm, a pressure differential across the diaphragm, a material for the diaphragm, stress and strain characteristics of the diaphragm and any combination thereof; or (ii) a peristaltic pumping system and the factor is selected from the group consisting of: inlet pressure to a peristaltic pumping tube, outlet pressure to the peristaltic pumping tube, material of the peristaltic pumping tube, tubing temperature, pumping head wear, tubing dimensions and any combination thereof.
